(19) United States
(12) Patent Application Publication
(10) Pub. No.: US 2025/0186477 A1
(43) Pub. Date: Jun. 12, 2025

(54) COMPOSITIONS COMPRISING FLUDARABINE PHOSPHATE AND METHODS OF MAKING AND USING SAME TO TREAT CANCER

(71) Applicant: Areva Pharmaceuticals Limited, Dublin (IE)

(72) Inventors: Victor Swaminathan, Louisville, KY (US); Soma Sekhar Reddy, Long Grove, IL (US)

(21) Appl. No.: 19/055,301

(22) Filed: Feb. 17, 2025

Related U.S. Application Data
(63) Continuation-in-part of application No. PCT/US24/11659, filed on Jan. 16, 2024.
(60) Provisional application No. 63/439,037, filed on Jan. 13, 2023.

(51) Int. Cl. A61K 31/7076; A61K 47/10; A61K 47/14; A61K 47/26

(52) U.S. Cl. CPC A61K 31/7076; A61K 47/10; A61K 47/14; A61K 47/26

(57) ABSTRACT

The present disclosure provides compositions (e.g., injectable compositions) comprising fludarabine (e.g., fludarabine phosphate), and methods of using same to treat cancers, such as a lymphoma, and/or to lymphodeplete a subject in need thereof, for example in association with a CAR-T therapeutic regimen.

### PRIORITY CLAIM

This application is a continuation-in-part application of International PCT Patent Application Serial No. PCT/US24/11659, filed on Jan. 16, 2024, which claims priority to U.S. Provisional Patent Application Ser. No. 63/439,037, filed on Jan. 13, 2023, the entire contents of each of which are incorporated herein by reference and relied upon.

### FIELD

This application provides compositions (e.g., injectable compositions) comprising fludarabine (e.g., fludarabine phosphate), and methods of using same to treat cancers, such as a lymphoma.

### BACKGROUND

Some cancers, including some lymphomas and some leukemias, can be treated by CAR-T therapeutic methods. Briefly, CAR-T therapy includes removing T cells from the subject, inserting genes encoding a chimeric antigen receptor (“CAR”) into the T cells, replicating the CAR T cells, and reintroducing the CAR-T cells into the subject. Often, the CAR-T therapy also includes lymphodepleting the subject before the CAR-T cells are administered to the subject. Fludarabine is known to act as a lymphodepletion agent useful in some CAR-T therapeutic methods.

When used in conjunction with known CAR-T therapeutic methods, however, fludarabine must be administered in an amount of 30 mg/m². At this dosage, common severe side effects include arm, back, or jaw pain; black, tarry stools; blood in the urine or stools; chest pain or discomfort; chest tightness or heaviness; constipation; cough or hoarseness; coughing or spitting up blood; fast or irregular heartbeat; fever or chills; general feeling of discomfort or illness; lower back or side pain; nausea; pain; painful, burning, or difficult urination; pale skin; pinpoint red spots on the skin; shortness of breath; sneezing; sore throat; sores, ulcers, or white spots on the lips or in the mouth; stomach pain, severe; sweating; swelling; tender, swollen glands in the neck; thickening of bronchial secretions; troubled breathing; unusual bleeding or bruising; unusual tiredness or weakness; vomiting of blood or material that looks like coffee grounds; and wheezing. Further, other studies show a troubling development of severe side effects at lower dosages of fludarabine, including high rates of pneumonia in refractory chronic lymphocytic leukemia patients receiving 22-40 mg/m² or 15-25 mg/m² fludarabine daily; and high rates of grade 11 or IV pulmonary toxicity (including Pneumocystis carinii pneumonia) in Non-Hodgkin's lymphoma patients receiving 20 mg/m² fludarabine with cyclophosphamide. Many other less severe and less common side effects have also been linked to fludarabine treatments.

In view of these clinically relevant shortcomings, a need exists for improved methods of CAR-T therapeutic methods.

### Objects of the Inventions

It is one object of the present inventions to provide an improved method of treating a lymphoma or a leukemia cancer.

It is another object of the present inventions to provide an improved CAR-T therapeutic method with reduced severity and/or incidence rate of side effects commonly associated with fludarabine lymphodepletion.

It is another object of the present inventions to provide an improved lymphodepletion method using fludarabine at a dosage not exceeding 25 mg/m².

It is another object of the present inventions to provide an improved lymphodepletion method that does not require administration of cyclophosphamide.

It is another object of the present inventions to provide an improved lymphodepletion method that does not require prior treatment with a protease inhibitor, an immunomodulatory drug, and/or an anti-CD-38 antibody.

It is another object of the present inventions to provide an improved ready-to-use fludarabine phosphate composition in a single-dose container.

It is another object of the present inventions to provide an improved ready-to-use fludarabine phosphate composition in a multi-dose container.

It is another object of the present inventions to provide an improved ready-to-use fludarabine composition having commercially-valuable shelf life.

It is another object of the present inventions to provide an improved ready-to-use fludarabine composition that does not include a preservative.

### SUMMARY

The present disclosure provides methods of treating cancer using fludarabine.

In some embodiments, the present disclosure provides a pharmaceutical product comprising: a single-use container; and a sterile composition housed within the single-use container, the composition including: 25 mg/mL fludarabine phosphate, 25 mg/mL mannitol, water for injection, an optional pH adjuster, an inert gaseous atmosphere, and an initial pH level of 6.5 to 7.1.

In other embodiments, the present disclosure provides a pharmaceutical product comprising: a single-use container; and a sterile composition housed within the single-use container, the composition including: 25 mg/mL fludarabine phosphate, 25 mg/mL sorbitol, water for injection, an optional pH adjuster, an inert gaseous atmosphere, and an initial pH level of 6.5 to 7.1.

In other embodiments, the present disclosure provides a pharmaceutical product comprising: a single-use container; and a sterile composition housed within the single-use container, the composition including: 30 mg/mL fludarabine phosphate, 40 mg/mL mannitol, 2.5 mg/mL methyl paraben, water for injection, an optional pH adjuster, an inert gaseous atmosphere, and an initial pH level of 6.5 to 7.1.

In other embodiments, the present disclosure provides a pharmaceutical product comprising: a single-use container; and a sterile composition housed within the single-use container, the composition including: 30 mg/mL fludarabine phosphate, 40 mg/mL sorbitol, 2.5 mg/mL methyl paraben, water for injection, an optional pH adjuster, an inert gaseous atmosphere, and an initial pH level of 6.5 to 7.1.

In other embodiments, the present disclosure provides a pharmaceutical product comprising: a multi-use container; and a sterile composition housed within the multi-use container, the composition including: 30 mg/mL fludarabine phosphate, 40 mg/mL mannitol, water for injection, an optional pH adjuster, an inert gaseous atmosphere, and an initial pH level of 6.5 to 7.1.

In other embodiments, the present disclosure provides a pharmaceutical product comprising: a multi-use container; and a sterile composition housed within the multi-use container, the composition including: 30 mg/mL fludarabine phosphate, 40 mg/mL sorbitol, water for injection, an optional pH adjuster, an inert gaseous atmosphere, and an initial pH level of 6.5 to 7.1.

In still other embodiments, the present disclosure provides a pharmaceutical product comprising: a multi-use container; and a sterile composition housed within the multi-use container, the composition including: fludarabine phosphate in an amount selected from the group consisting of: 25 mg/mL and 30 mg/mL, mannitol in an amount selected from the group consisting of: 0 mg/mL, 25 mg/mL and 40 mg/mL, sorbitol in an amount selected from the group consisting of: 0 mg/mL and 40 mg/mL, methyl paraben in an amount selected from the group consisting of: 0 mg/mL and 1.8 mg/mL, propyl paraben in an amount selected from the group consisting of: 0 mg/mL and 0.2 mg/mL, benzyl alcohol in an amount selected from the group consisting of: 0 mg/mL and 25 mg/mL, water for injection, an optional pH adjuster, an inert gaseous atmosphere, and an initial pH level of 6.5 to 7.1.

In other embodiments, the present disclosure provides a method of treating a lymphoma or a leukemia in a subject in need thereof, the method comprising: lymphodepleting the subject by administering an amount of fludarabine not exceeding about 25 mg/m²; and thereafter administering CAR-T cells to the subject.

In other embodiments, the present disclosure provides a method of lymphodepleting a subject, the method comprising administering to the subject an amount of fludarabine not exceeding about 25 mg/m².

These and other embodiments are described in further detail herein below.

The detailed description and examples provided herewith depict various embodiments of this disclosure for purposes of illustration only. One skilled in the art will readily recognize from the following discussion that alternative embodiments of the structures and methods illustrated herein may be employed without departing from the principles of embodiments described herein.

### DETAILED DESCRIPTION

The present disclosure provides methods of treating cancer by administering fludarabine.

The present disclosure is based in part on the inventor's discovery that lymphodepleting a subject in need of CAR-T therapy may be accomplished by administering fludarabine at a subtherapeutic dosage level (e.g., less than 30 mg/m²).

### Fludarabine and Fludarabine Phosphate

Compositions of the present disclosure comprise fludarabine and/or fludarabine phosphate.

Fludarabine ((2R,3S,4S,5R)-2-(6-amino-2-fluoropurin-9-yl)-5-(hydroxymethyl)oxolane-3,4-diol; CAS 21679-14-1) is an antineoplastic agent indicated for leukemias and lymphomas. It has an empirical formula of C₁₀H₁₂FN₅O₄ and a molecular mass of 285.24 g/mol. Fludarabine has a chemical structure as shown below:

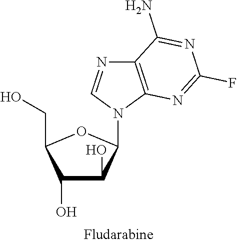

Fludarabine phosphate (CAS 75607-67-9) is the 5-O-phosphorylated form of fludarabine and is commercially available in the United States under the brand name Fludara. Fludarabine phosphate is also referred to as 9H-purin-6-amine,2-fluoro-9-(5-O-phosphono-β-D-arabinofuranosyl)(2-fluoro-ara-AMP) or ((2R,3S,4S,5R)-5-(6-amino-2-fluoro-9H-purin-9-yl)-3,4-dihydroxytetrahydrofuran-2-yl)methyl dihydrogen phosphate. Fludarabine phosphate has an empirical formula of C₁₀H₁₃FN₅O₇P, a molecular mass of 365.21 g/mol, and a chemical structure as shown below:

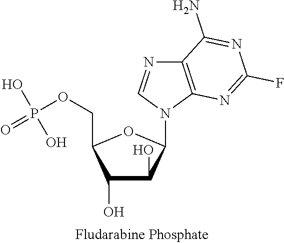

In some embodiments, a composition of the present disclosure comprises fludarabine. In some embodiments, the fludarabine is enantiopure (2R,3S,4S,5R)-2-(6-amino-2-fluoropurin-9-yl)-5-(hydroxymethyl)oxolane-3,4-diol, or substantially enantiopure (2R,3S,4S,5R)-2-(6-amino-2-fluoropurin-9-yl)-5-(hydroxymethyl)oxolane-3,4-diol.

In some embodiments, a composition of the present disclosure comprises fludarabine phosphate. In some embodiments, the fludarabine is enantiopure 9H-purin-6-amine,2-fluoro-9-(5-O-phosphono-β-D-arabinofuranosyl)(2-fluoro-ara-AMP), or substantially enantiopure 9H-purin-6-amine,2-fluoro-9-(5-O-phosphono-β-D-arabinofuranosyl)(2-fluoro-ara-AMP).

In some embodiments, a composition of the present disclosure comprises fludarabine or fludarabine phosphate, a tonicity agent, a solvent system, and optionally one or more pH adjusters to achieve an initial pH level of about 6.5 to about 7.1.

For example and without limitation, a composition of the present disclosure in one embodiment comprises fludarabine phosphate, mannitol, water (e.g., water for injection), and trace sodium hydroxide to achieve an initial pH of about 6.5 to about 7.1.

In some embodiments, compositions of the present disclosure comprise an amount of fludarabine phosphate not greater than about 25 mg/mL. For example and without limitation, compositions of the present disclosure comprise fludarabine phosphate in an amount of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, or about 25 mg/mL.

The tonicity agent may be any suitable tonicity agent soluble in the solvent system. In some embodiments, the tonicity agent comprises, consists essentially of, or consists of mannitol. In such embodiments, the mannitol may be present in an amount of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, or about 60 mg/mL. In some embodiments, mannitol is present in an amount of about 25 mg/mL. In some embodiments, mannitol is present in an amount of about 40 mg/mL.

In some embodiments, the tonicity agent comprises, consists essentially of, or consists of sorbitol. In such embodiments, the sorbitol may be present in an amount of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, or about 60 mg/mL. In some embodiments, sorbitol is present in an amount of about 25 mg/mL. In some embodiments, sorbitol is present in an amount of about 40 mg/mL.

The solvent system may comprise, consist essentially of, or consist of water (e.g., water for injection). In some embodiments, the solvent system consists essentially of water. In some embodiments, the solvent system consists of water.

The pH adjuster may be present in an amount sufficient to achieve an initial pH level of about 6.5 to about 7.1. In some embodiments, the pH adjuster is present, if at all, in an amount sufficient to achieve an initial pH level of about 6.6 to about 7.0. In some embodiments, the pH adjuster is present, if at all, in an amount sufficient to achieve an initial pH level of about 6.7 to about 6.9. In some embodiments, the pH adjuster is present, if at all, in an amount sufficient to achieve an initial pH level of about 6.5, about 6.6, about 6.7, about 6.8, about 6.9, about 7.0, or about 7.1. In some embodiments, the pH adjuster includes a base such as sodium hydroxide. In some embodiments, the pH adjuster includes an acid such as hydrochloric acid or acetic acid.

In some embodiments, compositions of the present disclosure include no preservative, such as an antimicrobial preservative. In some embodiments, a composition of the present disclosure includes no methyl paraben.

In some embodiments, a composition of the present disclosure comprises not more than 25 mg/mL fludarabine phosphate, about 25 mg/mL mannitol, water, and optionally a pH adjuster to achieve an initial pH level of about 6.5 to about 7.1.

In some embodiments, a composition of the present disclosure consists essentially of not more than 25 mg/mL fludarabine phosphate, about 25 mg/mL mannitol, water, and optionally a pH adjuster to achieve an initial pH level of about 6.5 to about 7.1.

In some embodiments, a composition of the present disclosure consists of not more than 25 mg/mL fludarabine phosphate, about 25 mg/mL mannitol, water, and optionally a pH adjuster to achieve an initial pH level of about 6.5 to about 7.1.

In some embodiments, a composition of the present disclosure comprises about 25 mg/mL fludarabine phosphate, about 25 mg/mL mannitol, water, and optionally a pH adjuster to achieve an initial pH level of about 6.5 to about 7.1.

In some embodiments, a composition of the present disclosure consists essentially of about 25 mg/mL fludarabine phosphate, about 25 mg/mL mannitol, water, and optionally a pH adjuster to achieve an initial pH level of about 6.5 to about 7.1.

In some embodiments, a composition of the present disclosure consists of about 25 mg/mL fludarabine phosphate, about 25 mg/mL mannitol, water, and optionally a pH adjuster to achieve an initial pH level of about 6.5 to about 7.1.

In some embodiments, a composition of the present disclosure comprises 50 mg fludarabine phosphate, 50 mg mannitol, optionally a pH adjuster to achieve an initial pH level of about 6.5 to about 7.1, and water to achieve a total volume of 2 mL.

In some embodiments, a composition of the present disclosure consists essentially of 50 mg fludarabine phosphate, 50 mg mannitol, optionally a pH adjuster to achieve an initial pH level of about 6.5 to about 7.1, and water to achieve a total volume of 2 mL.

In some embodiments, a composition of the present disclosure consists of 50 mg fludarabine phosphate, 50 mg mannitol, optionally a pH adjuster to achieve an initial pH level of about 6.5 to about 7.1, and water to achieve a total volume of 2 mL.

In some embodiments, compositions of the present disclosure include not more than about 2% total impurities, such as not more than about 2% total impurities, not more than about 1.9% total impurities, not more than about 1.8% total impurities, not more than about 1.7% total impurities, not more than about 1.6% total impurities, not more than about 1.5% total impurities, not more than about 1.4% total impurities, not more than about 1.3% total impurities, not more than about 1.2% total impurities, not more than about 1.1% total impurities, not more than about 1% total impurities, not more than about 0.9% total impurities, not more than about 0.8% total impurities, not more than about 0.7% total impurities, not more than about 0.6% total impurities, not more than about 0.5% total impurities, not more than about 0.4% total impurities, not more than about 0.3% total impurities, not more than about 0.2% total impurities, or not more than about 0.1% total impurities.

Iso-ara-guanine monophosphate (C₁₀H₁₄N₅O₈P; molecular weight 363.22; CAS 62314-92-5) is a common product of fludarabine degradation sometimes referred to as “fludarabine phosphate impurity A” and has the following structure:

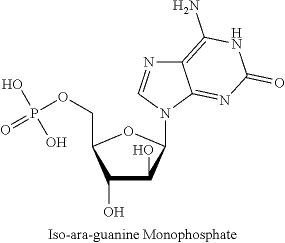

In some embodiments, compositions of the present disclosure include not more than about 1% iso-ara-guanine monophosphate impurity, such as not more than about 1% iso-ara-guanine monophosphate impurity, not more than about 0.9% iso-ara-guanine monophosphate impurity, not more than about 0.8% iso-ara-guanine monophosphate impurity, not more than about 0.7% iso-ara-guanine monophosphate impurity, not more than about 0.6% iso-ara-guanine monophosphate impurity, not more than about 0.5% iso-ara-guanine monophosphate impurity, not more than about 0.4% iso-ara-guanine monophosphate impurity, not more than about 0.3% iso-ara-guanine monophosphate impurity, not more than about 0.2% iso-ara-guanine monophosphate impurity, or not more than about 0.1% iso-ara-guanine monophosphate impurity.

In some embodiments, compositions of the present disclosure include not more than about 0.5% isoguanine impurity, such as not more than about 0.5% isoguanine monophosphate impurity, not more than about 0.4% isoguanine impurity, not more than about 0.3% isoguanine impurity, not more than about 0.2% isoguanine impurity, or not more than about 0.1% isoguanine impurity.

In some embodiments, compositions of the present disclosure include not more than about 0.5% 2-fluoroadenine impurity, such as not more than about 0.5% 2-fluoroadenine impurity, not more than about 0.4% 2-fluoroadenine impurity, not more than about 0.3% 2-fluoroadenine impurity, not more than about 0.2% 2-fluoroadenine impurity, or not more than about 0.1% 2-fluoroadenine impurity.

2-Fluoro-ara-adenine (C₁₀H₁₂FN₅O₄; molecular weight 285.23; CAS 146-78-1) is a common fludarabine impurity also referred to as 2-fluoro-9-β-D-ribofuranosyladenine, and has the following structure:

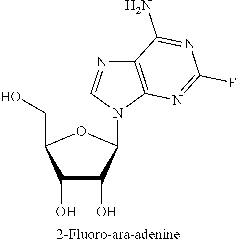

In some embodiments, compositions of the present disclosure include not more than about 0.5% 2-fluoro-ara-adenine impurity, such as not more than about 0.5% 2-fluoro-ara-adenine impurity, not more than about 0.4% 2-fluoro-ara-adenine impurity, not more than about 0.3% 2-fluoro-ara-adenine impurity, not more than about 0.2% 2-fluoro-ara-adenine impurity, or not more than about 0.1% 2-fluoro-ara-adenine impurity.

In some embodiments, compositions of the present disclosure include not more than about 0.5% of any impurity other than iso-ara-guanine monophosphate, isoguanine, 2-fluoroadenine, or 2-fluoro-ara-adenine, such as not more than about 0.5%, not more than about 0.4%, not more than about 0.3%, not more than about 0.2%, or not more than about 0.1%.

In some embodiments, compositions of the present disclosure have a pH level, after storage for a period of time at about 5° C., within 0.5 pH units of the initial pH level of the composition (e.g., when initially prepared). In some embodiments, the period of time is at least 6 months. In other embodiments, the period of time is at least 12 months. In other embodiments, the period of time is at least 18 months. In other embodiments, the period of time is at least 24 months. In other embodiments, the period of time is at least 30 months. In other embodiments, the period of time is at least 36 months. In other embodiments, the period of time is at least 42 months. In some embodiments, the period of time is at least 48 months.

Compositions consistent with the present disclosure may be stored in a suitable container closure system prior to use. In some embodiments, the container closure system comprises a vial, a stopper, and a seal.

The vial may be a glass vial, such as a type I glass vial.

The stopper may be a rubber stopper, such as a bromobutyl rubber stopper.

The seal may be a flip-off cap, such as an aluminum flip-off cap.

In some embodiments, headspace in the vial may comprise, consist essentially of, or consists of one or more inert gases, such as nitrogen, argon, or the like.

In some embodiments, a composition with the present disclosure is stable to pH drift over time at refrigerated storage conditions. For example and without limitation, a composition of the present invention may have a pH level after storage at about 5° C. for at least 3 months that is within about 0.5 pH units of an initial pH level of the composition. In some embodiments, the composition has a pH level after storage at about 5° C. for at least 3 months that is within about 0.4 pH units of an initial pH level of the composition. In some embodiments, the composition has a pH level after storage at about 5° C. for at least 3 months that is within about 0.3 pH units of an initial pH level of the composition. In some embodiments, the composition has a pH level after storage at about 5° C. for at least 3 months that is within about 0.2 pH units of an initial pH level of the composition. In some embodiments, the composition has a pH level after storage at about 5° C. for at least 3 months that is within about 0.1 pH units of an initial pH level of the composition. In some embodiments, the composition has a pH level after storage at about 5° C. for at least 3 months that is substantially the same as an initial pH level of the composition.

In some embodiments, a composition consistent with the present disclosure has a pH level after storage at about 5° C. for at least 3 months that is within about 0.5 pH units of an initial pH level of the composition. In some embodiments, the composition has a pH level after storage at about 5° C. for at least 4 months that is within about 0.5 pH units of an initial pH level of the composition. In some embodiments, the composition has a pH level after storage at about 5° C. for at least 5 months that is within about 0.5 pH units of an initial pH level of the composition. In some embodiments, the composition has a pH level after storage at about 5° C. for at least 6 months that is within about 0.5 pH units of an initial pH level of the composition. In some embodiments, the composition has a pH level after storage at about 5° C. for at least 7 months that is within about 0.5 pH units of an initial pH level of the composition. In some embodiments, the composition has a pH level after storage at about 5° C. for at least 8 months that is within about 0.5 pH units of an initial pH level of the composition. In some embodiments, the composition has a pH level after storage at about 5° C. for at least 9 months that is within about 0.5 pH units of an initial pH level of the composition. In some embodiments, the composition has a pH level after storage at about 5° C. for at least 10 months that is within about 0.5 pH units of an initial pH level of the composition. In some embodiments, the composition has a pH level after storage at about 5° C. for at least 11 months that is within about 0.5 pH units of an initial pH level of the composition. In some embodiments, the composition has a pH level after storage at about 5° C. for at least 12 months that is within about 0.5 pH units of an initial pH level of the composition. In some embodiments, the composition has a pH level after storage at about 5° C. for at least 13 months that is within about 0.5 pH units of an initial pH level of the composition. In some embodiments, the composition has a pH level after storage at about 5° C. for at least 14 months that is within about 0.5 pH units of an initial pH level of the composition. In some embodiments, the composition has a pH level after storage at about 5° C. for at least 15 months that is within about 0.5 pH units of an initial pH level of the composition. In some embodiments, the composition has a pH level after storage at about 5° C. for at least 16 months that is within about 0.5 pH units of an initial pH level of the composition. In some embodiments, the composition has a pH level after storage at about 5° C. for at least 17 months that is within about 0.5 pH units of an initial pH level of the composition. In some embodiments, the composition has a pH level after storage at about 5° C. for at least 18 months that is within about 0.5 pH units of an initial pH level of the composition. In some embodiments, the composition has a pH level after storage at about 5° C. for at least 19 months that is within about 0.5 pH units of an initial pH level of the composition. In some embodiments, the composition has a pH level after storage at about 5° C. for at least 20 months that is within about 0.5 pH units of an initial pH level of the composition. In some embodiments, the composition has a pH level after storage at about 5° C. for at least 21 months that is within about 0.5 pH units of an initial pH level of the composition. In some embodiments, the composition has a pH level after storage at about 5° C. for at least 22 months that is within about 0.5 pH units of an initial pH level of the composition. In some embodiments, the composition has a pH level after storage at about 5° C. for at least 23 months that is within about 0.5 pH units of an initial pH level of the composition. In some embodiments, the composition has a pH level after storage at about 5° C. for at least 24 months that is within about 0.5 pH units of an initial pH level of the composition. In some embodiments, the composition has a pH level after storage at about 5° C. for more than 24 months that is within about 0.5 pH units of an initial pH level of the composition.

In some embodiments, a composition with the present disclosure is stable to pH drift over time at accelerated storage conditions. For example and without limitation, a composition of the present invention may have a pH level after storage at about 25° C. and about 60% RH for at least 1 month that is within about 0.5 pH units of an initial pH level of the composition. In some embodiments, the composition has a pH level after storage at about 25° C. and about 60% RH for at least 1 month that is within about 0.4 pH units of an initial pH level of the composition. In some embodiments, the composition has a pH level after storage at about 25° C. and about 60% RH for at least 1 month that is within about 0.3 pH units of an initial pH level of the composition. In some embodiments, the composition has a pH level after storage at about 25° C. and about 60% RH for at least 1 month that is within about 0.2 pH units of an initial pH level of the composition. In some embodiments, the composition has a pH level after storage at about 25° C. and about 60% RH for at least 1 month that is within about 0.1 pH units of an initial pH level of the composition. In some embodiments, the composition has a pH level after storage at about 25° C. and about 60% RH for at least 1 month that is substantially the same as an initial pH level of the composition.

In some embodiments, a composition consistent with the present disclosure has a pH level after storage at about 25° C. and about 60% RH for at least 1 month that is within about 0.5 pH units of an initial pH level of the composition. In some embodiments, the composition has a pH level after storage at about 25° C. and about 60% RH for at least 2 months that is within about 0.5 pH units of an initial pH level of the composition. In some embodiments, the composition has a pH level after storage at about 25° C. and about 60% RH for at least 3 months that is within about 0.5 pH units of an initial pH level of the composition. In some embodiments, the composition has a pH level after storage at about 25° C. and about 60% RH for at least 6 months that is within about 0.5 pH units of an initial pH level of the composition. In some embodiments, the composition has a pH level after storage at about 25° C. and about 60% RH for more than 6 months that is within about 0.5 pH units of an initial pH level of the composition.

In some embodiments, a composition with the present disclosure is stable to API degradation over time at refrigerated storage conditions. For example and without limitation, a composition of the present invention may have a fludarabine (e.g., fludarabine and/or fludarabine phosphate) level after storage at about 5° C. for at least 3 months that is within about 5% of an initial fludarabine (e.g., fludarabine and/or fludarabine phosphate) level of the composition. In some embodiments, the composition has a fludarabine level after storage at about 5° C. for at least 3 months that is within about 4% of an initial fludarabine level of the composition. In some embodiments, the composition has a fludarabine level after storage at about 5° C. for at least 3 months that is within about 3% of an initial fludarabine level of the composition.

In some embodiments, the composition has a fludarabine level after storage at about 5° C. for at least 3 months that is within about 2% of an initial fludarabine level of the composition. In some embodiments, the composition has a fludarabine level after storage at about 5° C. for at least 3 months that is within about 1% of an initial fludarabine level of the composition.

In some embodiments, a composition consistent with the present disclosure has a fludarabine (e.g., fludarabine and/or fludarabine phosphate) level after storage at about 5° C. for at least 3 months that is within about 5% of an initial fludarabine (e.g., fludarabine and/or fludarabine phosphate) level of the composition. In some embodiments, the composition has a fludarabine level after storage at about 5° C. for at least 4 months that is within about 5% of an initial fludarabine level of the composition. In some embodiments, the composition has a fludarabine level after storage at about 5° C. for at least 5 months that is within about 5% of an initial fludarabine level of the composition. In some embodiments, the composition has a fludarabine level after storage at about 5° C. for at least 6 months that is within about 5% of an initial fludarabine level of the composition. In some embodiments, the composition has a fludarabine level after storage at about 5° C. for at least 7 months that is within about 5% of an initial fludarabine level of the composition. In some embodiments, the composition has a fludarabine level after storage at about 5° C. for at least 8 months that is within about 5% of an initial fludarabine level of the composition. In some embodiments, the composition has a fludarabine level after storage at about 5° C. for at least 9 months that is within about 5% of an initial fludarabine level of the composition. In some embodiments, the composition has a fludarabine level after storage at about 5° C. for at least 10 months that is within about 5% of an initial fludarabine level of the composition. In some embodiments, the composition has a fludarabine level after storage at about 5° C. for at least 11 months that is within about 5% of an initial fludarabine level of the composition. In some embodiments, the composition has a fludarabine level after storage at about 5° C. for at least 12 months that is within about 5% of an initial fludarabine level of the composition. In some embodiments, the composition has a fludarabine level after storage at about 5° C. for at least 13 months that is within about 5% of an initial fludarabine level of the composition. In some embodiments, the composition has a fludarabine level after storage at about 5° C. for at least 14 months that is within about 5% of an initial fludarabine level of the composition. In some embodiments, the composition has a fludarabine level after storage at about 5° C. for at least 15 months that is within about 5% of an initial fludarabine level of the composition. In some embodiments, the composition has a fludarabine level after storage at about 5° C. for at least 16 months that is within about 5% of an initial fludarabine level of the composition. In some embodiments, the composition has a fludarabine level after storage at about 5° C. for at least 17 months that is within about 5% of an initial fludarabine level of the composition. In some embodiments, the composition has a fludarabine level after storage at about 5° C. for at least 18 months that is within about 5% of an initial fludarabine level of the composition. In some embodiments, the composition has a fludarabine level after storage at about 5° C. for at least 19 months that is within about 5% of an initial fludarabine level of the composition. In some embodiments, the composition has a fludarabine level after storage at about 5° C. for at least 20 months that is within about 5% of an initial fludarabine level of the composition. In some embodiments, the composition has a fludarabine level after storage at about 5° C. for at least 21 months that is within about 5% of an initial fludarabine level of the composition. In some embodiments, the composition has a fludarabine level after storage at about 5° C. for at least 22 months that is within about 5% of an initial fludarabine level of the composition. In some embodiments, the composition has a fludarabine level after storage at about 5° C. for at least 23 months that is within about 5% of an initial fludarabine level of the composition. In some embodiments, the composition has a fludarabine level after storage at about 5° C. for at least 24 months that is within about 5% of an initial fludarabine level of the composition. In some embodiments, the composition has a fludarabine level after storage at about 5° C. for more than 24 months that is within about 5% of an initial fludarabine level of the composition.

In some embodiments, a composition with the present disclosure is stable to API degradation over time at accelerated storage conditions. For example and without limitation, a composition of the present invention may have a fludarabine (e.g., fludarabine and/or fludarabine phosphate) level after storage at about 25° C. and about 60% RH for at least 1 month that is within about 5% of an initial fludarabine (e.g., fludarabine and/or fludarabine phosphate) level of the composition. In some embodiments, the composition has a fludarabine level after storage at about 25° C. and about 60% RH for at least 1 month that is within about 4% of an initial fludarabine level of the composition. In some embodiments, the composition has a fludarabine level after storage at about 25° C. and about 60% RH for at least 1 month that is within about 3% of an initial fludarabine level of the composition. In some embodiments, the composition has a fludarabine level after storage at about 25° C. and about 60% RH for at least 1 month that is within about 2% of an initial fludarabine level of the composition. In some embodiments, the composition has a fludarabine level after storage at about 25° C. and about 60% RH for at least 1 month that is within about 1% of an initial fludarabine level of the composition.

In some embodiments, a composition consistent with the present disclosure has a fludarabine (e.g., fludarabine and/or fludarabine phosphate) level after storage at about 25° C. and about 60% RH for at least 1 month that is within about 5% of an initial fludarabine (e.g., fludarabine and/or fludarabine phosphate) level of the composition. In some embodiments, the composition has a fludarabine level after storage at about 25° C. and about 60% RH for at least 2 months that is within about 5% of an initial fludarabine level of the composition. In some embodiments, the composition has a fludarabine level after storage at about 25° C. and about 60% RH for at least 3 months that is within about 5% of an initial fludarabine level of the composition. In some embodiments, the composition has a fludarabine level after storage at about 25° C. and about 60% RH for at least 6 months that is within about 5% of an initial fludarabine level of the composition. In some embodiments, the composition has a fludarabine level after storage at about 25° C. and about 60% RH for more than 6 months that is within about 5% of an initial fludarabine level of the composition.

In some embodiments, a composition with the present disclosure is stable to impurity formation over time at refrigerated storage conditions. For example and without limitation, a composition of the present invention may have a level of total impurities after storage at about 5° C. for at least 3 months that is not more than about 5%. In some embodiments, the composition has a level of total impurities after storage at about 5° C. for at least 3 months that is not more than about 4%. In some embodiments, the composition has a level of total impurities after storage at about 5° C. for at least 3 months that is not more than about 3%. In some embodiments, the composition has a level of total impurities after storage at about 5° C. for at least 3 months that is not more than about 2%. In some embodiments, the composition has a level of total impurities after storage at about 5° C. for at least 3 months that is not more than about 1%.

In some embodiments, a composition consistent with the present disclosure has a level of total impurities after storage at about 5° C. for at least 3 months that is not more than about 2%. In some embodiments, the composition has a level of total impurities after storage at about 5° C. for at least 4 months that is not more than about 2%. In some embodiments, the composition has a level of total impurities after storage at about 5° C. for at least 5 months that is not more than about 2%. In some embodiments, the composition has a level of total impurities after storage at about 5° C. for at least 6 months that is not more than about 2%. In some embodiments, the composition has a level of total impurities after storage at about 5° C. for at least 7 months that is not more than about 2%. In some embodiments, the composition has a level of total impurities after storage at about 5° C. for at least 8 months that is not more than about 2%. In some embodiments, the composition has a level of total impurities after storage at about 5° C. for at least 9 months that is not more than about 2%. In some embodiments, the composition has a level of total impurities after storage at about 5° C. for at least 10 months that is not more than about 2%. In some embodiments, the composition has a level of total impurities after storage at about 5° C. for at least 11 months that is not more than about 2%. In some embodiments, the composition has a level of total impurities after storage at about 5° C. for at least 12 months that is not more than about 2%. In some embodiments, the composition has a level of total impurities after storage at about 5° C. for at least 13 months that is not more than about 2%. In some embodiments, the composition has a level of total impurities after storage at about 5° C. for at least 14 months that is not more than about 2%. In some embodiments, the composition has a level of total impurities after storage at about 5° C. for at least 15 months that is not more than about 2%. In some embodiments, the composition has a level of total impurities after storage at about 5° C. for at least 16 months that is not more than about 2%. In some embodiments, the composition has a level of total impurities after storage at about 5° C. for at least 17 months that is not more than about 2%. In some embodiments, the composition has a level of total impurities after storage at about 5° C. for at least 18 months that is not more than about 2%. In some embodiments, the composition has a level of total impurities after storage at about 5° C. for at least 19 months that is not more than about 2%. In some embodiments, the composition has a level of total impurities after storage at about 5° C. for at least 20 months that is not more than about 2%. In some embodiments, the composition has a level of total impurities after storage at about 5° C. for at least 21 months that is not more than about 2%. In some embodiments, the composition has a level of total impurities after storage at about 5° C. for at least 22 months that is not more than about 2%. In some embodiments, the composition has a level of total impurities after storage at about 5° C. for at least 23 months that is not more than about 2%. In some embodiments, the composition has a level of total impurities after storage at about 5° C. for at least 24 months that is not more than about 2%. In some embodiments, the composition has a level of total impurities after storage at about 5° C. for more than 24 months that is not more than about 2%.

In some embodiments, a composition with the present disclosure is stable to impurity formation over time at accelerated storage conditions. For example and without limitation, a composition of the present invention may have a level of total impurities after storage at about 25° C. and about 60% RH for at least 1 month that is not more than about 5%. In some embodiments, the composition has a level of total impurities after storage at about 25° C. and about 60% RH for at least 1 month that is not more than about 4%. In some embodiments, the composition has a level of total impurities after storage at about 25° C. and about 60% RH for at least 1 month that is not more than about 3%. In some embodiments, the composition has a level of total impurities after storage at about 25° C. and about 60% RH for at least 1 month that is not more than about 2%. In some embodiments, the composition has a level of total impurities after storage at about 25° C. and about 60% RH for at least 1 month that is not more than about 1%.

In some embodiments, a composition consistent with the present disclosure has a level of total impurities after storage at about 25° C. and about 60% RH for at least 1 month that is not more than about 2%. In some embodiments, the composition has a level of total impurities after storage at about 25° C. and about 60% RH for at least 2 months that is not more than about 2%. In some embodiments, the composition has a level of total impurities after storage at about 25° C. and about 60% RH for at least 3 months that is not more than about 2%. In some embodiments, the composition has a level of total impurities after storage at about 25° C. and about 60% RH for at least 6 months that is not more than about 2%. In some embodiments, the composition has a level of total impurities after storage at about 25° C. and about 60% RH for more than 6 months that is not more than about 2%.

In some embodiments, a composition with the present disclosure is stable to iso-ara-guanine monophosphate formation over time at refrigerated storage conditions. For example and without limitation, a composition of the present invention may have an iso-ara-guanine monophosphate level after storage at about 5° C. for at least 3 months that is not more than about 5%. In some embodiments, the composition has an iso-ara-guanine monophosphate level after storage at about 5° C. for at least 3 months that is not more than about 4%. In some embodiments, the composition has an iso-ara-guanine monophosphate level after storage at about 5° C. for at least 3 months that is not more than about 3%. In some embodiments, the composition has an iso-ara-guanine monophosphate level after storage at about 5° C. for at least 3 months that is not more than about 2%. In some embodiments, the composition has an iso-ara-guanine monophosphate level after storage at about 5° C. for at least 3 months that is not more than about 1%.

In some embodiments, a composition consistent with the present disclosure has an iso-ara-guanine monophosphate level after storage at about 5° C. for at least 3 months that is not more than about 1%. In some embodiments, the composition has an iso-ara-guanine monophosphate level after storage at about 5° C. for at least 4 months that is not more than about 1%. In some embodiments, the composition has an iso-ara-guanine monophosphate level after storage at about 5° C. for at least 5 months that is not more than about 1%. In some embodiments, the composition has an iso-ara-guanine monophosphate level after storage at about 5° C. for at least 6 months that is not more than about 1%. In some embodiments, the composition has an iso-ara-guanine monophosphate level after storage at about 5° C. for at least 7 months that is not more than about 1%. In some embodiments, the composition has an iso-ara-guanine monophosphate level after storage at about 5° C. for at least 8 months that is not more than about 1%. In some embodiments, the composition has an iso-ara-guanine monophosphate level after storage at about 5° C. for at least 9 months that is not more than about 1%. In some embodiments, the composition has an iso-ara-guanine monophosphate level after storage at about 5° C. for at least 10 months that is not more than about 1%. In some embodiments, the composition has an iso-ara-guanine monophosphate level after storage at about 5° C. for at least 11 months that is not more than about 1%. In some embodiments, the composition has an iso-ara-guanine monophosphate level after storage at about 5° C. for at least 12 months that is not more than about 1%. In some embodiments, the composition has an iso-ara-guanine monophosphate level after storage at about 5° C. for at least 13 months that is not more than about 1%. In some embodiments, the composition has an iso-ara-guanine monophosphate level after storage at about 5° C. for at least 14 months that is not more than about 1%. In some embodiments, the composition has an iso-ara-guanine monophosphate level after storage at about 5° C. for at least 15 months that is not more than about 1%. In some embodiments, the composition has an iso-ara-guanine monophosphate level after storage at about 5° C. for at least 16 months that is not more than about 1%. In some embodiments, the composition has an iso-ara-guanine monophosphate level after storage at about 5° C. for at least 7 months that is not more than about 1%. In some embodiments, the composition has an iso-ara-guanine monophosphate level after storage at about 5° C. for at least 18 months that is not more than about 1%. In some embodiments, the composition has an iso-ara-guanine monophosphate level after storage at about 5° C. for at least 19 months that is not more than about 1%. In some embodiments, the composition has an iso-ara-guanine monophosphate level after storage at about 5° C. for at least 20 months that is not more than about 1%. In some embodiments, the composition has an iso-ara-guanine monophosphate level after storage at about 5° C. for at least 21 months that is not more than about 1%. In some embodiments, the composition has an iso-ara-guanine monophosphate level after storage at about 5° C. for at least 22 months that is not more than about 1%. In some embodiments, the composition has an iso-ara-guanine monophosphate level after storage at about 5° C. for at least 23 months that is not more than about 1%. In some embodiments, the composition has an iso-ara-guanine monophosphate level after storage at about 5° C. for at least 24 months that is not more than about 1%. In some embodiments, the composition has an iso-ara-guanine monophosphate level after storage at about 5° C. for more than 24 months that is not more than about 1%.

In some embodiments, a composition with the present disclosure is stable to iso-ara-guanine monophosphate formation over time at accelerated storage conditions. For example and without limitation, a composition of the present invention may have an iso-ara-guanine monophosphate level after storage at about 25° C. and about 60% RH for at least 1 month that is not more than about 5%. In some embodiments, the composition has an iso-ara-guanine monophosphate level after storage at about 25° C. and about 60% RH for at least 1 month that is not more than about 4%. In some embodiments, the composition has an iso-ara-guanine monophosphate level after storage at about 25° C. and about 60% RH for at least 1 month that is not more than about 3%. In some embodiments, the composition has an iso-ara-guanine monophosphate level after storage at about 25° C. and about 60% RH for at least 1 month that is not more than about 2%. In some embodiments, the composition has an iso-ara-guanine monophosphate level after storage at about 25° C. and about 60% RH for at least 1 month that is not more than about 1%.

In some embodiments, a composition consistent with the present disclosure has an iso-ara-guanine monophosphate level after storage at about 25° C. and about 60% RH for at least 1 month that is not more than about 1%. In some embodiments, the composition has an iso-ara-guanine monophosphate level after storage at about 25° C. and about 60% RH for at least 2 months that is not more than about 1%. In some embodiments, the composition has an iso-ara-guanine monophosphate level after storage at about 25° C. and about 60% RH for at least 3 months that is not more than about 1%. In some embodiments, the composition has an iso-ara-guanine monophosphate level after storage at about 25° C. and about 60% RH for at least 6 months that is not more than about 1%. In some embodiments, the composition has an iso-ara-guanine monophosphate level after storage at about 25° C. and about 60% RH for more than 6 months that is not more than about 1%.

In some embodiments, a composition with the present disclosure is stable to isoguanine formation over time at refrigerated storage conditions. For example and without limitation, a composition of the present invention may have an isoguanine level after storage at about 5° C. for at least 3 months that is not more than about 1%. In some embodiments, the composition has an isoguanine level after storage at about 5° C. for at least 3 months that is not more than about 0.8%. In some embodiments, the composition has an isoguanine level after storage at about 5° C. for at least 3 months that is not more than about 0.6%. In some embodiments, the composition has an isoguanine level after storage at about 5° C. for at least 3 months that is not more than about 0.4%. In some embodiments, the composition has an isoguanine level after storage at about 5° C. for at least 3 months that is not more than about 0.2%. In some embodiments, the composition has an isoguanine level after storage at about 5° C. for at least 3 months that is not more than about 0.1%.

In some embodiments, a composition consistent with the present disclosure has an isoguanine level after storage at about 5° C. for at least 3 months that is not more than about 0.2%. In some embodiments, the composition has an isoguanine level after storage at about 5° C. for at least 4 months that is not more than about 0.2%. In some embodiments, the composition has an isoguanine level after storage at about 5° C. for at least 5 months that is not more than about 0.2%. In some embodiments, the composition has an isoguanine level after storage at about 5° C. for at least 6 months that is not more than about 0.2%. In some embodiments, the composition has an isoguanine level after storage at about 5° C. for at least 7 months that is not more than about 0.2%. In some embodiments, the composition has an isoguanine level after storage at about 5° C. for at least 8 months that is not more than about 0.2%. In some embodiments, the composition has an isoguanine level after storage at about 5° C. for at least 9 months that is not more than about 0.2%. In some embodiments, the composition has an isoguanine level after storage at about 5° C. for at least 10 months that is not more than about 0.2%. In some embodiments, the composition has an isoguanine level after storage at about 5° C. for at least 11 months that is not more than about 0.2%. In some embodiments, the composition has an isoguanine level after storage at about 5° C. for at least 12 months that is not more than about 0.2%. In some embodiments, the composition has an isoguanine level after storage at about 5° C. for at least 13 months that is not more than about 0.2%. In some embodiments, the composition has an isoguanine level after storage at about 5° C. for at least 14 months that is not more than about 0.2%. In some embodiments, the composition has an isoguanine level after storage at about 5° C. for at least 15 months that is not more than about 0.2%. In some embodiments, the composition has an isoguanine level after storage at about 5° C. for at least 16 months that is not more than about 0.2%. In some embodiments, the composition has an isoguanine level after storage at about 5° C. for at least 7 months that is not more than about 0.2%. In some embodiments, the composition has an isoguanine level after storage at about 5° C. for at least 18 months that is not more than about 0.2%. In some embodiments, the composition has an isoguanine level after storage at about 5° C. for at least 19 months that is not more than about 0.2%. In some embodiments, the composition has an isoguanine level after storage at about 5° C. for at least 20 months that is not more than about 0.2%. In some embodiments, the composition has an isoguanine level after storage at about 5° C. for at least 21 months that is not more than about 0.2%. In some embodiments, the composition has an isoguanine level after storage at about 5° C. for at least 22 months that is not more than about 0.2%. In some embodiments, the composition has an isoguanine level after storage at about 5° C. for at least 23 months that is not more than about 0.2%. In some embodiments, the composition has an isoguanine level after storage at about 5° C. for at least 24 months that is not more than about 0.2%. In some embodiments, the composition has an isoguanine level after storage at about 5° C. for more than 24 months that is not more than about 0.2%.

In some embodiments, a composition with the present disclosure is stable to isoguanine formation over time at accelerated storage conditions. For example and without limitation, a composition of the present invention may have an isoguanine level after storage at about 25° C. and about 60% RH for at least 1 month that is not more than about 1%. In some embodiments, the composition has an isoguanine level after storage at about 25° C. and about 60% RH for at least 1 month that is not more than about 0.8%. In some embodiments, the composition has an isoguanine level after storage at about 25° C. and about 60% RH for at least 1 month that is not more than about 0.6%. In some embodiments, the composition has an isoguanine level after storage at about 25° C. and about 60% RH for at least 1 month that is not more than about 0.4%. In some embodiments, the composition has an isoguanine level after storage at about 25° C. and about 60% RH for at least 1 month that is not more than about 0.2%. In some embodiments, the composition has an isoguanine level after storage at about 25° C. and about 60% RH for at least 1 month that is not more than about 0.1%.

In some embodiments, a composition consistent with the present disclosure has an isoguanine level after storage at about 25° C. and about 60% RH for at least 1 month that is not more than about 0.2%. In some embodiments, the composition has an isoguanine level after storage at about 25° C. and about 60% RH for at least 2 months that is not more than about 0.2%. In some embodiments, the composition has an isoguanine level after storage at about 25° C. and about 60% RH for at least 3 months that is not more than about 0.2%. In some embodiments, the composition has an isoguanine level after storage at about 25° C. and about 60% RH for at least 6 months that is not more than about 0.2%. In some embodiments, the composition has an isoguanine level after storage at about 25° C. and about 60% RH for more than 6 months that is not more than about 0.2%.

In some embodiments, a composition with the present disclosure is stable to 2-fluoroadenine formation over time at refrigerated storage conditions. For example and without limitation, a composition of the present invention may have a 2-fluoroadenine level after storage at about 5° C. for at least 3 months that is not more than about 1%. In some embodiments, the composition has a 2-fluoroadenine level after storage at about 5° C. for at least 3 months that is not more than about 0.8%. In some embodiments, the composition has a 2-fluoroadenine level after storage at about 5° C. for at least 3 months that is not more than about 0.6%. In some embodiments, the composition has a 2-fluoroadenine level after storage at about 5° C. for at least 3 months that is not more than about 0.4%. In some embodiments, the composition has a 2-fluoroadenine level after storage at about 5° C. for at least 3 months that is not more than about 0.2%. In some embodiments, the composition has a 2-fluoroadenine level after storage at about 5° C. for at least 3 months that is not more than about 0.1%.

In some embodiments, a composition consistent with the present disclosure has a 2-fluoroadenine level after storage at about 5° C. for at least 3 months that is not more than about 0.2%. In some embodiments, the composition has a 2-fluoroadenine level after storage at about 5° C. for at least 4 months that is not more than about 0.2%. In some embodiments, the composition has a 2-fluoroadenine level after storage at about 5° C. for at least 5 months that is not more than about 0.2%. In some embodiments, the composition has a 2-fluoroadenine level after storage at about 5° C. for at least 6 months that is not more than about 0.2%. In some embodiments, the composition has a 2-fluoroadenine level after storage at about 5° C. for at least 7 months that is not more than about 0.2%. In some embodiments, the composition has a 2-fluoroadenine level after storage at about 5° C. for at least 8 months that is not more than about 0.2%. In some embodiments, the composition has a 2-fluoroadenine level after storage at about 5° C. for at least 9 months that is not more than about 0.2%. In some embodiments, the composition has a 2-fluoroadenine level after storage at about 5° C. for at least 10 months that is not more than about 0.2%. In some embodiments, the composition has a 2-fluoroadenine level after storage at about 5° C. for at least 11 months that is not more than about 0.2%. In some embodiments, the composition has a 2-fluoroadenine level after storage at about 5° C. for at least 12 months that is not more than about 0.2%. In some embodiments, the composition has a 2-fluoroadenine level after storage at about 5° C. for at least 13 months that is not more than about 0.2%. In some embodiments, the composition has a 2-fluoroadenine level after storage at about 5° C. for at least 14 months that is not more than about 0.2%. In some embodiments, the composition has a 2-fluoroadenine level after storage at about 5° C. for at least 15 months that is not more than about 0.2%. In some embodiments, the composition has a 2-fluoroadenine level after storage at about 5° C. for at least 16 months that is not more than about 0.2%. In some embodiments, the composition has a 2-fluoroadenine level after storage at about 5° C. for at least 7 months that is not more than about 0.2%. In some embodiments, the composition has a 2-fluoroadenine level after storage at about 5° C. for at least 18 months that is not more than about 0.2%. In some embodiments, the composition has a 2-fluoroadenine level after storage at about 5° C. for at least 19 months that is not more than about 0.2%. In some embodiments, the composition has a 2-fluoroadenine level after storage at about 5° C. for at least 20 months that is not more than about 0.2%. In some embodiments, the composition has a 2-fluoroadenine level after storage at about 5° C. for at least 21 months that is not more than about 0.2%. In some embodiments, the composition has a 2-fluoroadenine level after storage at about 5° C. for at least 22 months that is not more than about 0.2%. In some embodiments, the composition has a 2-fluoroadenine level after storage at about 5° C. for at least 23 months that is not more than about 0.2%. In some embodiments, the composition has a 2-fluoroadenine level after storage at about 5° C. for at least 24 months that is not more than about 0.2%. In some embodiments, the composition has a 2-fluoroadenine level after storage at about 5° C. for more than 24 months that is not more than about 0.2%.

In some embodiments, a composition with the present disclosure is stable to 2-fluoroadenine formation over time at accelerated storage conditions. For example and without limitation, a composition of the present invention may have a 2-fluoroadenine level after storage at about 25° C. and about 60% RH for at least 1 month that is not more than about 1%. In some embodiments, the composition has a 2-fluoroadenine level after storage at about 25° C. and about 60% RH for at least 1 month that is not more than about 0.8%. In some embodiments, the composition has a 2-fluoroadenine level after storage at about 25° C. and about 60% RH for at least 1 month that is not more than about 0.6%. In some embodiments, the composition has a 2-fluoroadenine level after storage at about 25° C. and about 60% RH for at least 1 month that is not more than about 0.4%. In some embodiments, the composition has a 2-fluoroadenine level after storage at about 25° C. and about 60% RH for at least 1 month that is not more than about 0.2%. In some embodiments, the composition has a 2-fluoroadenine level after storage at about 25° C. and about 60% RH for at least 1 month that is not more than about 0.1%.

In some embodiments, a composition consistent with the present disclosure has a 2-fluoroadenine level after storage at about 25° C. and about 60% RH for at least 1 month that is not more than about 0.2%. In some embodiments, the composition has a 2-fluoroadenine level after storage at about 25° C. and about 60% RH for at least 2 months that is not more than about 0.2%. In some embodiments, the composition has a 2-fluoroadenine level after storage at about 25° C. and about 60% RH for at least 3 months that is not more than about 0.2%. In some embodiments, the composition has a 2-fluoroadenine level after storage at about 25° C. and about 60% RH for at least 6 months that is not more than about 0.2%. In some embodiments, the composition has a 2-fluoroadenine level after storage at about 25° C. and about 60% RH for more than 6 months that is not more than about 0.2%.

In some embodiments, a composition with the present disclosure is stable to 2-fluoro-ara-adenine formation over time at refrigerated storage conditions. For example and without limitation, a composition of the present invention may have a 2-fluoro-ara-adenine level after storage at about 5° C. for at least 3 months that is not more than about 1%. In some embodiments, the composition has a 2-fluoro-ara-adenine level after storage at about 5° C. for at least 3 months that is not more than about 0.8%. In some embodiments, the composition has a 2-fluoro-ara-adenine level after storage at about 5° C. for at least 3 months that is not more than about 0.6%. In some embodiments, the composition has a 2-fluoro-ara-adenine level after storage at about 5° C. for at least 3 months that is not more than about 0.4%. In some embodiments, the composition has a 2-fluoro-ara-adenine level after storage at about 5° C. for at least 3 months that is not more than about 0.2%. In some embodiments, the composition has a 2-fluoro-ara-adenine level after storage at about 5° C. for at least 3 months that is not more than about 0.1%.

In some embodiments, a composition consistent with the present disclosure has a 2-fluoro-ara-adenine level after storage at about 5° C. for at least 3 months that is not more than about 0.2%. In some embodiments, the composition has a 2-fluoro-ara-adenine level after storage at about 5° C. for at least 4 months that is not more than about 0.2%. In some embodiments, the composition has a 2-fluoro-ara-adenine level after storage at about 5° C. for at least 5 months that is not more than about 0.2%. In some embodiments, the composition has a 2-fluoro-ara-adenine level after storage at about 5° C. for at least 6 months that is not more than about 0.2%. In some embodiments, the composition has a 2-fluoro-ara-adenine level after storage at about 5° C. for at least 7 months that is not more than about 0.2%. In some embodiments, the composition has a 2-fluoro-ara-adenine level after storage at about 5° C. for at least 8 months that is not more than about 0.2%. In some embodiments, the composition has a 2-fluoro-ara-adenine level after storage at about 5° C. for at least 9 months that is not more than about 0.2%. In some embodiments, the composition has a 2-fluoro-ara-adenine level after storage at about 5° C. for at least 10 months that is not more than about 0.2%. In some embodiments, the composition has a 2-fluoro-ara-adenine level after storage at about 5° C. for at least 11 months that is not more than about 0.2%. In some embodiments, the composition has a 2-fluoro-ara-adenine level after storage at about 5° C. for at least 12 months that is not more than about 0.2%. In some embodiments, the composition has a 2-fluoro-ara-adenine level after storage at about 5° C. for at least 13 months that is not more than about 0.2%. In some embodiments, the composition has a 2-fluoro-ara-adenine level after storage at about 5° C. for at least 14 months that is not more than about 0.2%. In some embodiments, the composition has a 2-fluoro-ara-adenine level after storage at about 5° C. for at least 15 months that is not more than about 0.2%. In some embodiments, the composition has a 2-fluoro-ara-adenine level after storage at about 5° C. for at least 16 months that is not more than about 0.2%. In some embodiments, the composition has a 2-fluoro-ara-adenine level after storage at about 5° C. for at least 7 months that is not more than about 0.2%. In some embodiments, the composition has a 2-fluoro-ara-adenine level after storage at about 5° C. for at least 18 months that is not more than about 0.2%. In some embodiments, the composition has a 2-fluoro-ara-adenine level after storage at about 5° C. for at least 19 months that is not more than about 0.2%. In some embodiments, the composition has a 2-fluoro-ara-adenine level after storage at about 5° C. for at least 20 months that is not more than about 0.2%. In some embodiments, the composition has a 2-fluoro-ara-adenine level after storage at about 5° C. for at least 21 months that is not more than about 0.2%. In some embodiments, the composition has a 2-fluoro-ara-adenine level after storage at about 5° C. for at least 22 months that is not more than about 0.2%. In some embodiments, the composition has a 2-fluoro-ara-adenine level after storage at about 5° C. for at least 23 months that is not more than about 0.2%. In some embodiments, the composition has a 2-fluoro-ara-adenine level after storage at about 5° C. for at least 24 months that is not more than about 0.2%. In some embodiments, the composition has a 2-fluoro-ara-adenine level after storage at about 5° C. for more than 24 months that is not more than about 0.2%.

In some embodiments, a composition with the present disclosure is stable to 2-fluoro-ara-adenine formation over time at accelerated storage conditions. For example and without limitation, a composition of the present invention may have a 2-fluoro-ara-adenine level after storage at about 25° C. and about 60% RH for at least 1 month that is not more than about 1%. In some embodiments, the composition has a 2-fluoro-ara-adenine level after storage at about 25° C. and about 60% RH for at least 1 month that is not more than about 0.8%. In some embodiments, the composition has a 2-fluoro-ara-adenine level after storage at about 25° C. and about 60% RH for at least 1 month that is not more than about 0.6%. In some embodiments, the composition has a 2-fluoro-ara-adenine level after storage at about 25° C. and about 60% RH for at least 1 month that is not more than about 0.4%. In some embodiments, the composition has a 2-fluoro-ara-adenine level after storage at about 25° C. and about 60% RH for at least 1 month that is not more than about 0.2%. In some embodiments, the composition has a 2-fluoro-ara-adenine level after storage at about 25° C. and about 60% RH for at least 1 month that is not more than about 0.1%.

In some embodiments, a composition consistent with the present disclosure has a 2-fluoro-ara-adenine level after storage at about 25° C. and about 60% RH for at least 1 month that is not more than about 0.2%. In some embodiments, the composition has a 2-fluoro-ara-adenine level after storage at about 25° C. and about 60% RH for at least 2 months that is not more than about 0.2%. In some embodiments, the composition has a 2-fluoro-ara-adenine level after storage at about 25° C. and about 60% RH for at least 3 months that is not more than about 0.2%. In some embodiments, the composition has a 2-fluoro-ara-adenine level after storage at about 25° C. and about 60% RH for at least 6 months that is not more than about 0.2%. In some embodiments, the composition has a 2-fluoro-ara-adenine level after storage at about 25° C. and about 60% RH for more than 6 months that is not more than about 0.2%.

In some embodiments, a composition with the present disclosure is stable to subvisible particle formation over time at refrigerated storage conditions. For example and without limitation, a composition of the present invention may have an amount of ≥10 μm particles after storage at about 5° C. for at least 3 months that is not more than 6000 per 2 mL vial. In some embodiments, the composition has an amount of ≥25 μm particles after storage at about 5° C. for at least 3 months that is not more than 600 per 2 mL vial. In some embodiments, the composition has an amount of ≥10 μm particles after storage at about 5° C. for at least 3 months that is not more than 6000 per 2 mL vial and an amount of ≥25 μm particles after storage at about 5° C. for at least 3 months that is not more than 600 per 2 mL vial.

In some embodiments, a composition with the present disclosure is stable to subvisible particle formation over time at accelerated storage conditions. For example and without limitation, a composition of the present invention may have an amount of ≥10 μm particles after storage at about 25° C. and about 60% RH for at least 1 month that is not more than 6000 per 2 mL vial. In some embodiments, the composition has an amount of 10 μm particles after storage at about 25° C. and about 60% RH for at least 2 months that is not more than 6000 per 2 mL vial. In some embodiments, the composition has an amount of ≥10 μm particles after storage at about 25° C. and about 60% RH for at least 3 months that is not more than 6000 per 2 mL vial. In some embodiments, the composition has an amount of ≥10 μm particles after storage at about 25° C. and about 60% RH for at least 6 months that is not more than 6000 per 2 mL vial. In some embodiments, the composition has an amount of ≥10 μm particles after storage at about 25° C. and about 60% RH for more than 6 months that is not more than 6000 per 2 mL vial.

In some embodiments, the composition has an amount of ≥25 μm particles after storage at about 25° C. and about 60% RH for at least 1 month that is not more than 600 per 2 mL vial. In some embodiments, the composition has an amount of ≥25 μm particles after storage at about 25° C. and about 60% RH for at least 2 months that is not more than 600 per 2 mL vial. In some embodiments, the composition has an amount of 25 μm particles after storage at about 25° C. and about 60% RH for at least 3 months that is not more than 600 per 2 mL vial. In some embodiments, the composition has an amount of ≥25 μm particles after storage at about 25° C. and about 60% RH for at least 6 months that is not more than 600 per 2 mL vial. In some embodiments, the composition has an amount of ≥25 μm particles after storage at about 25° C. and about 60% RH for more than 6 months that is not more than 600 per 2 mL vial.

In some embodiments, the composition has an amount of ≥10 μm particles after storage at about 25° C. and about 60% RH for at least 1 month that is not more than 6000 per 2 mL vial and an amount of ≥25 μm particles after storage at about 25° C. and about 60% RH for at least 1 month that is not more than 600 per 2 mL vial. In some embodiments, the composition has an amount of ≥10 μm particles after storage at about 25° C. and about 60% RH for at least 2 months that is not more than 6000 per 2 mL vial and an amount of ≥25 μm particles after storage at about 25° C. and about 60% RH for at least 2 months that is not more than 600 per 2 mL vial. In some embodiments, the composition has an amount of ≥10 μm particles after storage at about 25° C. and about 60% RH for at least 3 months that is not more than 6000 per 2 mL vial and an amount of ≥25 μm particles after storage at about 25° C. and about 60% RH for at least 3 months that is not more than 600 per 2 mL vial. In some embodiments, the composition has an amount of ≥10 μm particles after storage at about 25° C. and about 60% RH for at least 6 months that is not more than 6000 per 2 mL vial and an amount of 25 μm particles after storage at about 25° C. and about 60% RH for at least 6 months that is not more than 600 per 2 mL vial. In some embodiments, the composition has an amount of ≥10 μm particles after storage at about 25° C. and about 60% RH for more than 6 months that is not more than 6000 per 2 mL vial and an amount of ≥25 μm particles after storage at about 25° C. and about 60% RH for more than 6 months that is not more than 600 per 2 mL vial.

In some embodiments, a composition of the present disclosure comprises not more than 25 mg/mL fludarabine phosphate, about 25 mg/mL mannitol, water, an initial pH level of about 6.5 to about 7.1, and, after storage at about 5° C. for at least 3 months, an amount of fludarabine (e.g., fludarabine and/or fludarabine phosphate) within 5% of the initial amount of fludarabine phosphate in the composition, and a pH level within 0.5 pH units of the initial pH level of the composition.

In some embodiments, a composition of the present disclosure comprises not more than 25 mg/mL fludarabine phosphate, about 25 mg/mL mannitol, water, an initial pH level of about 6.5 to about 7.1, and, after storage at about 5° C. for at least 6 months, an amount of fludarabine (e.g., fludarabine and/or fludarabine phosphate) within 5% of the initial amount of fludarabine phosphate in the composition, and a pH level within 0.5 pH units of the initial pH level of the composition.

In some embodiments, a composition of the present disclosure comprises not more than 25 mg/mL fludarabine phosphate, about 25 mg/mL mannitol, water, an initial pH level of about 6.5 to about 7.1, and, after storage at about 5° C. for at least 9 months, an amount of fludarabine (e.g., fludarabine and/or fludarabine phosphate) within 5% of the initial amount of fludarabine phosphate in the composition, and a pH level within 0.5 pH units of the initial pH level of the composition.

In some embodiments, a composition of the present disclosure comprises not more than 25 mg/mL fludarabine phosphate, about 25 mg/mL mannitol, water, an initial pH level of about 6.5 to about 7.1, and, after storage at about 5° C. for at least 18 months, an amount of fludarabine (e.g., fludarabine and/or fludarabine phosphate) within 5% of the initial amount of fludarabine phosphate in the composition, and a pH level within 0.5 pH units of the initial pH level of the composition.

In some embodiments, a composition of the present disclosure comprises not more than 25 mg/mL fludarabine phosphate, about 25 mg/mL mannitol, water, an initial pH level of about 6.5 to about 7.1, and, after storage at about 5° C. for at least 24 months, an amount of fludarabine (e.g., fludarabine and/or fludarabine phosphate) within 5% of the initial amount of fludarabine phosphate in the composition, and a pH level within 0.5 pH units of the initial pH level of the composition.

In some embodiments, a composition of the present disclosure comprises not more than 25 mg/mL fludarabine phosphate, about 25 mg/mL mannitol, water, an initial pH level of about 6.5 to about 7.1, and, after storage at about 5° C. for more than 24 months, an amount of fludarabine (e.g., fludarabine and/or fludarabine phosphate) within 5% of the initial amount of fludarabine phosphate in the composition, and a pH level within 0.5 pH units of the initial pH level of the composition.

In some embodiments, a composition of the present disclosure comprises not more than 25 mg/mL fludarabine phosphate, about 25 mg/mL mannitol, water, an initial pH level of about 6.5 to about 7.1, and, after storage at about 25° C. and about 60% RH for at least 1 month, an amount of fludarabine (e.g., fludarabine and/or fludarabine phosphate) within 5% of the initial amount of fludarabine phosphate in the composition, and a pH level within 0.5 pH units of the initial pH level of the composition.

In some embodiments, a composition of the present disclosure comprises not more than 25 mg/mL fludarabine phosphate, about 25 mg/mL mannitol, water, an initial pH level of about 6.5 to about 7.1, and, after storage at about 25° C. and about 60% RH for at least 2 months, an amount of fludarabine (e.g., fludarabine and/or fludarabine phosphate) within 5% of the initial amount of fludarabine phosphate in the composition, and a pH level within 0.5 pH units of the initial pH level of the composition.

In some embodiments, a composition of the present disclosure comprises not more than 25 mg/mL fludarabine phosphate, about 25 mg/mL mannitol, water, an initial pH level of about 6.5 to about 7.1, and, after storage at about 25° C. and about 60% RH for at least 3 months, an amount of fludarabine (e.g., fludarabine and/or fludarabine phosphate) within 5% of the initial amount of fludarabine phosphate in the composition, and a pH level within 0.5 pH units of the initial pH level of the composition.

In some embodiments, a composition of the present disclosure comprises not more than 25 mg/mL fludarabine phosphate, about 25 mg/mL mannitol, water, an initial pH level of about 6.5 to about 7.1, and, after storage at about 25° C. and about 60% RH for at least 6 months, an amount of fludarabine (e.g., fludarabine and/or fludarabine phosphate) within 5% of the initial amount of fludarabine phosphate in the composition, and a pH level within 0.5 pH units of the initial pH level of the composition.

In some embodiments, a composition of the present disclosure comprises not more than 25 mg/mL fludarabine phosphate, about 25 mg/mL mannitol, water, an initial pH level of about 6.5 to about 7.1, and, after storage at about 25° C. and about 60% RH for more than 6 months, an amount of fludarabine (e.g., fludarabine and/or fludarabine phosphate) within 5% of the initial amount of fludarabine phosphate in the composition, and a pH level within 0.5 pH units of the initial pH level of the composition.

In some embodiments, a preservative-free fludarabine phosphate composition consistent with the present disclosure comprises 25 mg/mL fludarabine phosphate, a tonicity agent, water, an initial pH level of about 6.5 to about 7.1, and, after storage at about 25° C. and about 60% RH for more than 6 months, a bacterial endotoxin level of not more than 7.7 EU per mg of fludarabine phosphate, such as not more than 7.7 EU per mg of fludarabine phosphate, not more than 7.6 EU per mg of fludarabine phosphate, not more than 7.5 EU per mg of fludarabine phosphate, not more than 7.4 EU per mg of fludarabine phosphate, not more than 7.3 EU per mg of fludarabine phosphate, not more than 7.2 EU per mg of fludarabine phosphate, not more than 7.1 EU per mg of fludarabine phosphate, not more than 7 EU per mg of fludarabine phosphate, not more than 6.9 EU per mg of fludarabine phosphate, not more than 6.8 EU per mg of fludarabine phosphate, not more than 6.7 EU per mg of fludarabine phosphate, not more than 6.6 EU per mg of fludarabine phosphate, not more than 6.5 EU per mg of fludarabine phosphate, not more than 6.4 EU per mg of fludarabine phosphate, not more than 6.3 EU per mg of fludarabine phosphate, not more than 6.2 EU per mg of fludarabine phosphate, not more than 6.1 EU per mg of fludarabine phosphate, not more than 6 EU per mg of fludarabine phosphate, not more than 5.9 EU per mg of fludarabine phosphate, not more than 5.8 EU per mg of fludarabine phosphate, not more than 5.7 EU per mg of fludarabine phosphate, not more than 5.6 EU per mg of fludarabine phosphate, not more than 5.5 EU per mg of fludarabine phosphate, not more than 5.4 EU per mg of fludarabine phosphate, not more than 5.3 EU per mg of fludarabine phosphate, not more than 5.2 EU per mg of fludarabine phosphate, not more than 5.1 EU per mg of fludarabine phosphate, not more than 5 EU per mg of fludarabine phosphate, not more than 4.9 EU per mg of fludarabine phosphate, not more than 4.8 EU per mg of fludarabine phosphate, not more than 4.7 EU per mg of fludarabine phosphate, not more than 4.6 EU per mg of fludarabine phosphate, not more than 4.5 EU per mg of fludarabine phosphate, not more than 4.4 EU per mg of fludarabine phosphate, not more than 4.3 EU per mg of fludarabine phosphate, not more than 4.2 EU per mg of fludarabine phosphate, not more than 4.1 EU per mg of fludarabine phosphate, not more than 4 EU per mg of fludarabine phosphate, not more than 3.9 EU per mg of fludarabine phosphate, not more than 3.8 EU per mg of fludarabine phosphate, not more than 3.7 EU per mg of fludarabine phosphate, not more than 3.6 EU per mg of fludarabine phosphate, not more than 3.5 EU per mg of fludarabine phosphate, not more than 3.4 EU per mg of fludarabine phosphate, not more than 3.3 EU per mg of fludarabine phosphate, not more than 3.2 EU per mg of fludarabine phosphate, not more than 3.1 EU per mg of fludarabine phosphate, not more than 3 EU per mg of fludarabine phosphate, not more than 2.9 EU per mg of fludarabine phosphate, not more than 2.8 EU per mg of fludarabine phosphate, not more than 2.7 EU per mg of fludarabine phosphate, not more than 2.6 EU per mg of fludarabine phosphate, not more than 2.5 EU per mg of fludarabine phosphate, not more than 2.4 EU per mg of fludarabine phosphate, not more than 2.3 EU per mg of fludarabine phosphate, not more than 2.2 EU per mg of fludarabine phosphate, not more than 2.1 EU per mg of fludarabine phosphate, not more than 2 EU per mg of fludarabine phosphate, not more than 1.9 EU per mg of fludarabine phosphate, not more than 1.8 EU per mg of fludarabine phosphate, not more than 1.7 EU per mg of fludarabine phosphate, not more than 1.6 EU per mg of fludarabine phosphate, not more than 1.5 EU per mg of fludarabine phosphate, not more than 1.4 EU per mg of fludarabine phosphate, not more than 1.3 EU per mg of fludarabine phosphate, not more than 1.2 EU per mg of fludarabine phosphate, not more than 1.1 EU per mg of fludarabine phosphate, not more than 1 EU per mg of fludarabine phosphate, not more than 0.9 EU per mg of fludarabine phosphate, not more than 0.8 EU per mg of fludarabine phosphate, not more than 0.7 EU per mg of fludarabine phosphate, not more than 0.6 EU per mg of fludarabine phosphate, not more than 0.5 EU per mg of fludarabine phosphate, not more than 0.4 EU per mg of fludarabine phosphate, not more than 0.3 EU per mg of fludarabine phosphate, not more than 0.2 EU per mg of fludarabine phosphate, or not more than 0.1 EU per mg of fludarabine phosphate. In some embodiments, the preservative-free fludarabine phosphate composition is a sterile preservative-free fludarabine phosphate composition before storage at about 25° C. and about 60% RH for more than 6 months. In some embodiments, the preservative-free fludarabine phosphate composition includes no methyl paraben. In some embodiments, the preservative-free fludarabine phosphate composition is stored at about 25° C. and about 60% RH for more than 6 months under an inert atmosphere, such as nitrogen. In some embodiments, the preservative-free fludarabine phosphate composition is stored in a single-use container (e.g., a single-use vial or a pre-filled syringe). In other embodiments, the preservative-free fludarabine phosphate composition is stored in a multi-use container. In some embodiments, the preservative-free fludarabine phosphate composition satisfies sterility criteria set forth in USP <71> (e.g., no evidence of microbial growth is observed) (e.g., no evidence of microbial growth is observed) after storage at about 25° C. and about 60% RH for more than 6 months. In some embodiments, the preservative-free fludarabine phosphate composition satisfies sterility criteria set forth in USP <51> (e.g., satisfies bacterial and/or yeast and mold tests for Category 1 Products) after storage at about 25° C. and about 60% RH for more than 6 months.

In some embodiments, a preservative-free fludarabine phosphate composition consistent with the present disclosure comprises 30 mg/mL fludarabine phosphate, a tonicity agent, water, an initial pH level of about 6.5 to about 7.1, and, after storage at about 25° C. and about 60% RH for more than 6 months, a bacterial endotoxin level of not more than 7.7 EU per mg of fludarabine phosphate, such as not more than 7.7 EU per mg of fludarabine phosphate, not more than 7.6 EU per mg of fludarabine phosphate, not more than 7.5 EU per mg of fludarabine phosphate, not more than 7.4 EU per mg of fludarabine phosphate, not more than 7.3 EU per mg of fludarabine phosphate, not more than 7.2 EU per mg of fludarabine phosphate, not more than 7.1 EU per mg of fludarabine phosphate, not more than 7 EU per mg of fludarabine phosphate, not more than 6.9 EU per mg of fludarabine phosphate, not more than 6.8 EU per mg of fludarabine phosphate, not more than 6.7 EU per mg of fludarabine phosphate, not more than 6.6 EU per mg of fludarabine phosphate, not more than 6.5 EU per mg of fludarabine phosphate, not more than 6.4 EU per mg of fludarabine phosphate, not more than 6.3 EU per mg of fludarabine phosphate, not more than 6.2 EU per mg of fludarabine phosphate, not more than 6.1 EU per mg of fludarabine phosphate, not more than 6 EU per mg of fludarabine phosphate, not more than 5.9 EU per mg of fludarabine phosphate, not more than 5.8 EU per mg of fludarabine phosphate, not more than 5.7 EU per mg of fludarabine phosphate, not more than 5.6 EU per mg of fludarabine phosphate, not more than 5.5 EU per mg of fludarabine phosphate, not more than 5.4 EU per mg of fludarabine phosphate, not more than 5.3 EU per mg of fludarabine phosphate, not more than 5.2 EU per mg of fludarabine phosphate, not more than 5.1 EU per mg of fludarabine phosphate, not more than 5 EU per mg of fludarabine phosphate, not more than 4.9 EU per mg of fludarabine phosphate, not more than 4.8 EU per mg of fludarabine phosphate, not more than 4.7 EU per mg of fludarabine phosphate, not more than 4.6 EU per mg of fludarabine phosphate, not more than 4.5 EU per mg of fludarabine phosphate, not more than 4.4 EU per mg of fludarabine phosphate, not more than 4.3 EU per mg of fludarabine phosphate, not more than 4.2 EU per mg of fludarabine phosphate, not more than 4.1 EU per mg of fludarabine phosphate, not more than 4 EU per mg of fludarabine phosphate, not more than 3.9 EU per mg of fludarabine phosphate, not more than 3.8 EU per mg of fludarabine phosphate, not more than 3.7 EU per mg of fludarabine phosphate, not more than 3.6 EU per mg of fludarabine phosphate, not more than 3.5 EU per mg of fludarabine phosphate, not more than 3.4 EU per mg of fludarabine phosphate, not more than 3.3 EU per mg of fludarabine phosphate, not more than 3.2 EU per mg of fludarabine phosphate, not more than 3.1 EU per mg of fludarabine phosphate, not more than 3 EU per mg of fludarabine phosphate, not more than 2.9 EU per mg of fludarabine phosphate, not more than 2.8 EU per mg of fludarabine phosphate, not more than 2.7 EU per mg of fludarabine phosphate, not more than 2.6 EU per mg of fludarabine phosphate, not more than 2.5 EU per mg of fludarabine phosphate, not more than 2.4 EU per mg of fludarabine phosphate, not more than 2.3 EU per mg of fludarabine phosphate, not more than 2.2 EU per mg of fludarabine phosphate, not more than 2.1 EU per mg of fludarabine phosphate, not more than 2 EU per mg of fludarabine phosphate, not more than 1.9 EU per mg of fludarabine phosphate, not more than 1.8 EU per mg of fludarabine phosphate, not more than 1.7 EU per mg of fludarabine phosphate, not more than 1.6 EU per mg of fludarabine phosphate, not more than 1.5 EU per mg of fludarabine phosphate, not more than 1.4 EU per mg of fludarabine phosphate, not more than 1.3 EU per mg of fludarabine phosphate, not more than 1.2 EU per mg of fludarabine phosphate, not more than 1.1 EU per mg of fludarabine phosphate, not more than 1 EU per mg of fludarabine phosphate, not more than 0.9 EU per mg of fludarabine phosphate, not more than 0.8 EU per mg of fludarabine phosphate, not more than 0.7 EU per mg of fludarabine phosphate, not more than 0.6 EU per mg of fludarabine phosphate, not more than 0.5 EU per mg of fludarabine phosphate, not more than 0.4 EU per mg of fludarabine phosphate, not more than 0.3 EU per mg of fludarabine phosphate, not more than 0.2 EU per mg of fludarabine phosphate, or not more than 0.1 EU per mg of fludarabine phosphate. In some embodiments, the preservative-free fludarabine phosphate composition is a sterile preservative-free fludarabine phosphate composition before storage at about 25° C. and about 60% RH for more than 6 months. In some embodiments, the preservative-free fludarabine phosphate composition includes no methyl paraben. In some embodiments, the preservative-free fludarabine phosphate composition is stored at about 25° C. and about 60% RH for more than 6 months under an inert atmosphere, such as nitrogen. In some embodiments, the preservative-free fludarabine phosphate composition is stored in a single-use container (e.g., a single-use vial or a pre-filled syringe). In other embodiments, the preservative-free fludarabine phosphate composition is stored in a multi-use container. In some embodiments, the preservative-free fludarabine phosphate composition satisfies sterility criteria set forth in USP <71> (e.g., no evidence of microbial growth is observed) after storage at about 25° C. and about 60% RH for more than 6 months. In some embodiments, the preservative-free fludarabine phosphate composition satisfies sterility criteria set forth in USP <51> (e.g., satisfies bacterial and/or yeast and mold tests for Category 1 Products) after storage at about 25° C. and about 60% RH for more than 6 months.

In some embodiments, a drug product consistent with the present disclosure comprises, consists essentially of, or consists of a single-use container including about 2 mL of a composition comprising 25 mg/mL fludarabine phosphate, 25 mg/mL of a tonicity agent such as mannitol or sorbitol, water q.s., an initial pH level of about 6.5 to about 7.1, an inert atmosphere comprising, consisting essentially of, or consisting of nitrogen, and a bacterial endotoxin level after storage at about 25° C. and about 60% RH for more than 6 months of not more than 7.7 EU per mg of fludarabine phosphate, such as not more than 7.7 EU per mg of fludarabine phosphate, not more than 7.6 EU per mg of fludarabine phosphate, not more than 7.5 EU per mg of fludarabine phosphate, not more than 7.4 EU per mg of fludarabine phosphate, not more than 7.3 EU per mg of fludarabine phosphate, not more than 7.2 EU per mg of fludarabine phosphate, not more than 7.1 EU per mg of fludarabine phosphate, not more than 7 EU per mg of fludarabine phosphate, not more than 6.9 EU per mg of fludarabine phosphate, not more than 6.8 EU per mg of fludarabine phosphate, not more than 6.7 EU per mg of fludarabine phosphate, not more than 6.6 EU per mg of fludarabine phosphate, not more than 6.5 EU per mg of fludarabine phosphate, not more than 6.4 EU per mg of fludarabine phosphate, not more than 6.3 EU per mg of fludarabine phosphate, not more than 6.2 EU per mg of fludarabine phosphate, not more than 6.1 EU per mg of fludarabine phosphate, not more than 6 EU per mg of fludarabine phosphate, not more than 5.9 EU per mg of fludarabine phosphate, not more than 5.8 EU per mg of fludarabine phosphate, not more than 5.7 EU per mg of fludarabine phosphate, not more than 5.6 EU per mg of fludarabine phosphate, not more than 5.5 EU per mg of fludarabine phosphate, not more than 5.4 EU per mg of fludarabine phosphate, not more than 5.3 EU per mg of fludarabine phosphate, not more than 5.2 EU per mg of fludarabine phosphate, not more than 5.1 EU per mg of fludarabine phosphate, not more than 5 EU per mg of fludarabine phosphate, not more than 4.9 EU per mg of fludarabine phosphate, not more than 4.8 EU per mg of fludarabine phosphate, not more than 4.7 EU per mg of fludarabine phosphate, not more than 4.6 EU per mg of fludarabine phosphate, not more than 4.5 EU per mg of fludarabine phosphate, not more than 4.4 EU per mg of fludarabine phosphate, not more than 4.3 EU per mg of fludarabine phosphate, not more than 4.2 EU per mg of fludarabine phosphate, not more than 4.1 EU per mg of fludarabine phosphate, not more than 4 EU per mg of fludarabine phosphate, not more than 3.9 EU per mg of fludarabine phosphate, not more than 3.8 EU per mg of fludarabine phosphate, not more than 3.7 EU per mg of fludarabine phosphate, not more than 3.6 EU per mg of fludarabine phosphate, not more than 3.5 EU per mg of fludarabine phosphate, not more than 3.4 EU per mg of fludarabine phosphate, not more than 3.3 EU per mg of fludarabine phosphate, not more than 3.2 EU per mg of fludarabine phosphate, not more than 3.1 EU per mg of fludarabine phosphate, not more than 3 EU per mg of fludarabine phosphate, not more than 2.9 EU per mg of fludarabine phosphate, not more than 2.8 EU per mg of fludarabine phosphate, not more than 2.7 EU per mg of fludarabine phosphate, not more than 2.6 EU per mg of fludarabine phosphate, not more than 2.5 EU per mg of fludarabine phosphate, not more than 2.4 EU per mg of fludarabine phosphate, not more than 2.3 EU per mg of fludarabine phosphate, not more than 2.2 EU per mg of fludarabine phosphate, not more than 2.1 EU per mg of fludarabine phosphate, not more than 2 EU per mg of fludarabine phosphate, not more than 1.9 EU per mg of fludarabine phosphate, not more than 1.8 EU per mg of fludarabine phosphate, not more than 1.7 EU per mg of fludarabine phosphate, not more than 1.6 EU per mg of fludarabine phosphate, not more than 1.5 EU per mg of fludarabine phosphate, not more than 1.4 EU per mg of fludarabine phosphate, not more than 1.3 EU per mg of fludarabine phosphate, not more than 1.2 EU per mg of fludarabine phosphate, not more than 1.1 EU per mg of fludarabine phosphate, not more than 1 EU per mg of fludarabine phosphate, not more than 0.9 EU per mg of fludarabine phosphate, not more than 0.8 EU per mg of fludarabine phosphate, not more than 0.7 EU per mg of fludarabine phosphate, not more than 0.6 EU per mg of fludarabine phosphate, not more than 0.5 EU per mg of fludarabine phosphate, not more than 0.4 EU per mg of fludarabine phosphate, not more than 0.3 EU per mg of fludarabine phosphate, not more than 0.2 EU per mg of fludarabine phosphate, or not more than 0.1 EU per mg of fludarabine phosphate. In some embodiments, the composition is sterile before storage at about 25° C. and about 60% RH for more than 6 months. In some embodiments, the composition includes an antimicrobial preservative such as methyl paraben. In other embodiments, the composition includes no preservative agent. In some embodiments, the fludarabine phosphate composition satisfies sterility criteria set forth in USP <71> (e.g., no evidence of microbial growth is observed) after storage at about 25° C. and about 60% RH for more than 6 months in the single-use container. In some embodiments, the fludarabine phosphate composition satisfies sterility criteria set forth in USP <51> (e.g., satisfies bacterial and/or yeast and mold tests for Category 1 Products) after storage at about 25° C. and about 60% RH for more than 6 months in the single-use container.

In some embodiments, a drug product consistent with the present disclosure comprises, consists essentially of, or consists of a multi-use container including about 5 mL of a composition comprising 25 mg/mL fludarabine phosphate, 40 mg/mL of a tonicity agent such as mannitol or sorbitol, water q.s., an initial pH level of about 6.5 to about 7.1, an inert atmosphere comprising, consisting essentially of, or consisting of nitrogen, and a bacterial endotoxin level after storage at about 25° C. and about 60% RH for more than 6 months of not more than 7.7 EU per mg of fludarabine phosphate, such as not more than 7.7 EU per mg of fludarabine phosphate, not more than 7.6 EU per mg of fludarabine phosphate, not more than 7.5 EU per mg of fludarabine phosphate, not more than 7.4 EU per mg of fludarabine phosphate, not more than 7.3 EU per mg of fludarabine phosphate, not more than 7.2 EU per mg of fludarabine phosphate, not more than 7.1 EU per mg of fludarabine phosphate, not more than 7 EU per mg of fludarabine phosphate, not more than 6.9 EU per mg of fludarabine phosphate, not more than 6.8 EU per mg of fludarabine phosphate, not more than 6.7 EU per mg of fludarabine phosphate, not more than 6.6 EU per mg of fludarabine phosphate, not more than 6.5 EU per mg of fludarabine phosphate, not more than 6.4 EU per mg of fludarabine phosphate, not more than 6.3 EU per mg of fludarabine phosphate, not more than 6.2 EU per mg of fludarabine phosphate, not more than 6.1 EU per mg of fludarabine phosphate, not more than 6 EU per mg of fludarabine phosphate, not more than 5.9 EU per mg of fludarabine phosphate, not more than 5.8 EU per mg of fludarabine phosphate, not more than 5.7 EU per mg of fludarabine phosphate, not more than 5.6 EU per mg of fludarabine phosphate, not more than 5.5 EU per mg of fludarabine phosphate, not more than 5.4 EU per mg of fludarabine phosphate, not more than 5.3 EU per mg of fludarabine phosphate, not more than 5.2 EU per mg of fludarabine phosphate, not more than 5.1 EU per mg of fludarabine phosphate, not more than 5 EU per mg of fludarabine phosphate, not more than 4.9 EU per mg of fludarabine phosphate, not more than 4.8 EU per mg of fludarabine phosphate, not more than 4.7 EU per mg of fludarabine phosphate, not more than 4.6 EU per mg of fludarabine phosphate, not more than 4.5 EU per mg of fludarabine phosphate, not more than 4.4 EU per mg of fludarabine phosphate, not more than 4.3 EU per mg of fludarabine phosphate, not more than 4.2 EU per mg of fludarabine phosphate, not more than 4.1 EU per mg of fludarabine phosphate, not more than 4 EU per mg of fludarabine phosphate, not more than 3.9 EU per mg of fludarabine phosphate, not more than 3.8 EU per mg of fludarabine phosphate, not more than 3.7 EU per mg of fludarabine phosphate, not more than 3.6 EU per mg of fludarabine phosphate, not more than 3.5 EU per mg of fludarabine phosphate, not more than 3.4 EU per mg of fludarabine phosphate, not more than 3.3 EU per mg of fludarabine phosphate, not more than 3.2 EU per mg of fludarabine phosphate, not more than 3.1 EU per mg of fludarabine phosphate, not more than 3 EU per mg of fludarabine phosphate, not more than 2.9 EU per mg of fludarabine phosphate, not more than 2.8 EU per mg of fludarabine phosphate, not more than 2.7 EU per mg of fludarabine phosphate, not more than 2.6 EU per mg of fludarabine phosphate, not more than 2.5 EU per mg of fludarabine phosphate, not more than 2.4 EU per mg of fludarabine phosphate, not more than 2.3 EU per mg of fludarabine phosphate, not more than 2.2 EU per mg of fludarabine phosphate, not more than 2.1 EU per mg of fludarabine phosphate, not more than 2 EU per mg of fludarabine phosphate, not more than 1.9 EU per mg of fludarabine phosphate, not more than 1.8 EU per mg of fludarabine phosphate, not more than 1.7 EU per mg of fludarabine phosphate, not more than 1.6 EU per mg of fludarabine phosphate, not more than 1.5 EU per mg of fludarabine phosphate, not more than 1.4 EU per mg of fludarabine phosphate, not more than 1.3 EU per mg of fludarabine phosphate, not more than 1.2 EU per mg of fludarabine phosphate, not more than 1.1 EU per mg of fludarabine phosphate, not more than 1 EU per mg of fludarabine phosphate, not more than 0.9 EU per mg of fludarabine phosphate, not more than 0.8 EU per mg of fludarabine phosphate, not more than 0.7 EU per mg of fludarabine phosphate, not more than 0.6 EU per mg of fludarabine phosphate, not more than 0.5 EU per mg of fludarabine phosphate, not more than 0.4 EU per mg of fludarabine phosphate, not more than 0.3 EU per mg of fludarabine phosphate, not more than 0.2 EU per mg of fludarabine phosphate, or not more than 0.1 EU per mg of fludarabine phosphate. In some embodiments, the composition is sterile before storage at about 25° C. and about 60% RH for more than 6 months. In some embodiments, the composition includes an antimicrobial preservative such as methyl paraben. In other embodiments, the composition includes no preservative agent. In some embodiments, the fludarabine phosphate composition satisfies sterility criteria set forth in USP <71> (e.g., no evidence of microbial growth is observed) after storage at about 25° C. and about 60% RH for more than 6 months in the multi-use container. In some embodiments, the fludarabine phosphate composition satisfies sterility criteria set forth in USP <51> (e.g., satisfies bacterial and/or yeast and mold tests for Category 1 Products) after storage at about 25° C. and about 60% RH for more than 6 months in the multi-use container.

In some embodiments, the present disclosure provides a fludarabine composition comprising 25 mg/mL fludarabine phosphate, 25 mg/mL mannitol, 0 mg/mL sorbitol, 0 mg/mL methyl paraben, water for injection, and an initial pH of 6.5 to 7.1.

In some embodiments, the present disclosure provides a fludarabine composition comprising 25 mg/mL fludarabine phosphate, 0 mg/mL mannitol, 25 mg/mL sorbitol, 0 mg/mL methyl paraben, water for injection, and an initial pH of 6.5 to 7.1.

In some embodiments, the present disclosure provides a fludarabine composition comprising 30 mg/mL fludarabine phosphate, 40 mg/mL mannitol, 0 mg/mL sorbitol, 2.5 mg/mL methyl paraben, water for injection, and an initial pH of 6.5 to 7.1.

In some embodiments, the present disclosure provides a fludarabine composition comprising 30 mg/mL fludarabine phosphate, 0 mg/mL mannitol, 40 mg/mL sorbitol, 2.5 mg/mL methyl paraben, water for injection, and an initial pH of 6.5 to 7.1.

In some embodiments, the present disclosure provides a fludarabine composition comprising 30 mg/mL fludarabine phosphate, 40 mg/mL mannitol, 0 mg/mL sorbitol, 0 mg/mL methyl paraben, water for injection, and an initial pH of 6.5 to 7.1.

In some embodiments, the present disclosure provides a fludarabine composition comprising 30 mg/mL fludarabine phosphate, 0 mg/mL mannitol, 40 mg/mL sorbitol, 0 mg/mL methyl paraben, water for injection, and an initial pH of 6.5 to 7.1.

In some embodiments, the present disclosure provides a stable pharmaceutical product in a single-use container, the product comprising a sterile composition within the single-use container including: 25 mg/mL fludarabine phosphate, 25 mg/mL mannitol, 0 mg/mL sorbitol, 0 mg/mL methyl paraben, water for injection, and an initial pH of 6.5 to 7.1.

In some embodiments, the present disclosure provides a stable pharmaceutical product in a single-use container, the product comprising a sterile composition within the single-use container including: 25 mg/mL fludarabine phosphate, 0 mg/mL mannitol, 25 mg/mL sorbitol, 0 mg/mL methyl paraben, water for injection, and an initial pH of 6.5 to 7.1.

In some embodiments, the present disclosure provides a stable pharmaceutical product in a multi-dose container, the product comprising a sterile composition within the multi-dose container including: 30 mg/mL fludarabine phosphate, 40 mg/mL mannitol, 0 mg/mL sorbitol, 2.5 mg/mL methyl paraben, water for injection, and an initial pH of 6.5 to 7.1.

In some embodiments, the present disclosure provides a stable pharmaceutical product in a multi-dose container, the product comprising a sterile composition within the multi-dose container including: 30 mg/mL fludarabine phosphate, 0 mg/mL mannitol, 40 mg/mL sorbitol, 2.5 mg/mL methyl paraben, water for injection, and an initial pH of 6.5 to 7.1.

In some embodiments, the present disclosure provides a preservative-free stable pharmaceutical product in a multi-dose container, the product comprising a sterile composition within the multi-dose container including: 30 mg/mL fludarabine phosphate, 40 mg/mL mannitol, 0 mg/mL sorbitol, 0 mg/mL methyl paraben, water for injection, and an initial pH of 6.5 to 7.1.

In some embodiments, the present disclosure provides a preservative-free stable pharmaceutical product in a multi-dose container, the product comprising a sterile composition within the multi-dose container including: 30 mg/mL fludarabine phosphate, 0 mg/mL mannitol, 40 mg/mL sorbitol, 0 mg/mL methyl paraben, water for injection, and an initial pH of 6.5 to 7.1.

In some embodiments, the composition is prepared by mixing the fludarabine phosphate, mannitol, methyl paraben, water, and an optional pH adjuster, and then sterilizing the mixture by, for example, filtering the mixture through a sterilization grade filter.

In some embodiments, the present disclosure provides a pharmaceutical product suitable for multiple uses (e.g., for injecting multiple doses into a single subject), the pharmaceutical product comprising: a multi-use container; and a sterile composition housed within the multi-use container, the composition including: fludarabine phosphate in an amount selected from the group consisting of: 25 mg/mL and 30 mg/mL, mannitol in an amount selected from the group consisting of: 0 mg/mL, 25 mg/mL and 40 mg/mL, sorbitol in an amount selected from the group consisting of: 0 mg/mL and 40 mg/mL, methyl paraben in an amount selected from the group consisting of: 0 mg/mL and 1.8 mg/mL, propyl paraben in an amount selected from the group consisting of: 0 mg/mL and 0.2 mg/mL, benzyl alcohol in an amount selected from the group consisting of: 0 mg/mL and 25 mg/mL, water for injection, an optional pH adjuster, an inert gaseous atmosphere, and an initial pH level of 6.5 to 7.1. After storage at 5° C.+/−3° C. for at least 12 months, the composition includes: not more than 1.0% iso-ara-guanine-monophosphate; not more than 0.2% isoguanine; not more than 0.2% of any other early-eluting impurity (e.g., an impurity other than iso-ara-guanine-monophosphate, isoguanine, or a 3.5-diphosphate analog that elutes by HPLC equipped with a C18 column (e.g., Phenomenex Luna® C18 (2), 5 μm, 150×4.6 mm), and a UV detector set to 260 nm wavelength, with a mobile phase equal to or similar to, for example, 6% v/v methanol in 0.01 M potassium dihydrogen phosphate at a flow rate at or near 1.0 mL/min and a column temperature at or near room temperature); not more than 0.2% 2-fluoroadenine; not more than 0.2% 2-fluoro-ara-adenine; and not more than 2.0% of any other late-eluting impurity (e.g., an impurity other than 2-fluoroadenine, 2-fluoro-ara-adenine, or a 2-ethoxyphosphate analog that elutes by HPLC equipped with a C18 column (e.g., Phenomenex Luna® C18 (2), 5 μm, 150-4.6 mm), and a UV detector set to 260 nm wavelength, a mobile phase equal to or similar to, for example, 20% v/v methanol in 0.01 M potassium dihydrogen phosphate at a flow rate at or near 1.0 mL/min and a column temperature at or near room temperature). After storage at 25° C. and 60% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. After storage at 40° C. and 75% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. In some embodiments, the pharmaceutical product is prepared by a method comprising: purging an amount of water for injection representing about 80% of a desired total composition volume with nitrogen gas; heating the purged water for injection to about 70° C.; adding methyl paraben and/or propyl paraben to the heated water for injection under continuous stirring; cooling the mixture of water for injection and methyl paraben and/or propyl paraben to room temperature; adding mannitol and/or sorbitol to the room temperature mixture under continuous stirring; adding fludarabine phosphate to the mixture of methyl paraben and/or propyl paraben, mannitol and/or sorbitol, and water for injection under continuous stirring; adding, if needed, an amount of sodium hydroxide to bring a pH of the fludarabine phosphate mixture to 6.8; adding, if needed, additional water for injection to achieve the desired total composition volume; stirring the mixture at the desired total composition volume; placing the stirred mixture into one or more storage containers; and sealing the storage containers under nitrogen blanketing. In some embodiments, the method of preparation further comprises sterilizing the stirred mixture before the step of placing the stirred mixture into one or more storage containers; in other embodiments, the method of preparation further comprises sterilizing the stirred mixture after the step of sealing the storage containers under nitrogen blanketing.

In some embodiments, the present disclosure provides a pharmaceutical product suitable for multiple uses (e.g., for injecting multiple doses into a single subject), the pharmaceutical product comprising: a multi-use container; and a sterile composition housed within the multi-use container, the composition including: fludarabine phosphate in an amount selected from the group consisting of: 25 mg/mL and 30 mg/mL, mannitol in an amount selected from the group consisting of: 0 mg/mL, 25 mg/mL and 40 mg/mL, sorbitol in an amount selected from the group consisting of: 0 mg/mL and 40 mg/mL, 0 mg/mL methyl paraben, 0 mg/mL propyl paraben, 0 mg/mL benzyl alcohol, water for injection, an optional pH adjuster, an inert gaseous atmosphere, and an initial pH level of 6.5 to 7.1. After storage at 5° C.+/−3° C. for at least 12 months, the composition includes: not more than 1.0% iso-ara-guanine-monophosphate; not more than 0.2% isoguanine; not more than 0.2% of any other early-eluting impurity; not more than 0.2% 2-fluoroadenine; not more than 0.2% 2-fluoro-ara-adenine; and not more than 2.0% of any other late-eluting impurity. After storage at 25° C. and 60% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. After storage at 40° C. and 75% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. In some embodiments, the pharmaceutical product is prepared by a method comprising: purging an amount of water for injection representing about 80% of a desired total composition volume with nitrogen gas; heating the purged water for injection to about 70° C.; adding methyl paraben and/or propyl paraben to the heated water for injection under continuous stirring; cooling the mixture of water for injection and methyl paraben and/or propyl paraben to room temperature; adding mannitol and/or sorbitol to the room temperature mixture under continuous stirring; adding fludarabine phosphate to the mixture of methyl paraben and/or propyl paraben, mannitol and/or sorbitol, and water for injection under continuous stirring; adding, if needed, an amount of sodium hydroxide to bring a pH of the fludarabine phosphate mixture to 6.8; adding, if needed, additional water for injection to achieve the desired total composition volume; stirring the mixture at the desired total composition volume; placing the stirred mixture into one or more storage containers; and sealing the storage containers under nitrogen blanketing. In some embodiments, the method of preparation further comprises sterilizing the stirred mixture before the step of placing the stirred mixture into one or more storage containers; in other embodiments, the method of preparation further comprises sterilizing the stirred mixture after the step of sealing the storage containers under nitrogen blanketing.

In some embodiments, the present disclosure provides a pharmaceutical product suitable for multiple uses (e.g., for injecting multiple doses into a single subject), the pharmaceutical product comprising: a multi-use container; and a sterile composition housed within the multi-use container, the composition including: fludarabine phosphate in an amount selected from the group consisting of: 25 mg/mL and 30 mg/mL, mannitol in an amount selected from the group consisting of: 0 mg/mL, 25 mg/mL and 40 mg/mL, 0 mg/mL sorbitol, methyl paraben in an amount selected from the group consisting of: 0 mg/mL and 1.8 mg/mL, propyl paraben in an amount selected from the group consisting of: 0 mg/mL and 0.2 mg/mL, benzyl alcohol in an amount selected from the group consisting of: 0 mg/mL and 25 mg/mL, water for injection, an optional pH adjuster, an inert gaseous atmosphere, and an initial pH level of 6.5 to 7.1. After storage at 5° C.+/−3° C. for at least 12 months, the composition includes: not more than 1.0% iso-ara-guanine-monophosphate; not more than 0.2% isoguanine; not more than 0.2% of any other early-eluting impurity; not more than 0.2% 2-fluoroadenine; not more than 0.2% 2-fluoro-ara-adenine; and not more than 2.0% of any other late-eluting impurity. After storage at 25° C. and 60% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. After storage at 40° C. and 75% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. In some embodiments, the pharmaceutical product is prepared by a method comprising: purging an amount of water for injection representing about 80% of a desired total composition volume with nitrogen gas; heating the purged water for injection to about 70° C.; adding methyl paraben and/or propyl paraben to the heated water for injection under continuous stirring; cooling the mixture of water for injection and methyl paraben and/or propyl paraben to room temperature; adding mannitol and/or sorbitol to the room temperature mixture under continuous stirring; adding fludarabine phosphate to the mixture of methyl paraben and/or propyl paraben, mannitol and/or sorbitol, and water for injection under continuous stirring; adding, if needed, an amount of sodium hydroxide to bring a pH of the fludarabine phosphate mixture to 6.8; adding, if needed, additional water for injection to achieve the desired total composition volume; stirring the mixture at the desired total composition volume; placing the stirred mixture into one or more storage containers; and sealing the storage containers under nitrogen blanketing. In some embodiments, the method of preparation further comprises sterilizing the stirred mixture before the step of placing the stirred mixture into one or more storage containers; in other embodiments, the method of preparation further comprises sterilizing the stirred mixture after the step of sealing the storage containers under nitrogen blanketing.

In some embodiments, the present disclosure provides a pharmaceutical product suitable for multiple uses (e.g., for injecting multiple doses into a single subject), the pharmaceutical product comprising: a multi-use container; and a sterile composition housed within the multi-use container, the composition including: 25 mg/mL fludarabine phosphate, 25 mg/mL mannitol, 0 mg/mL sorbitol, 1.8 mg/mL methyl paraben, 0.2 mg/mL propyl paraben, 0 mg/mL benzyl alcohol, water for injection, an optional pH adjuster, an inert gaseous atmosphere, and an initial pH level of 6.5 to 7.1. After storage at 5° C.+/−3° C. for at least 12 months, the composition includes: not more than 1.0% iso-ara-guanine-monophosphate; not more than 0.2% isoguanine; not more than 0.2% of any other early-eluting impurity; not more than 0.2% 2-fluoroadenine; not more than 0.2% 2-fluoro-ara-adenine; and not more than 2.0% of any other late-eluting impurity. After storage at 25° C. and 60% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. After storage at 40° C. and 75% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. In some embodiments, the pharmaceutical product is prepared by a method comprising: purging an amount of water for injection representing about 80% of a desired total composition volume with nitrogen gas; heating the purged water for injection to about 70° C.; adding methyl paraben and/or propyl paraben to the heated water for injection under continuous stirring; cooling the mixture of water for injection and methyl paraben and/or propyl paraben to room temperature; adding mannitol and/or sorbitol to the room temperature mixture under continuous stirring; adding fludarabine phosphate to the mixture of methyl paraben and/or propyl paraben, mannitol and/or sorbitol, and water for injection under continuous stirring; adding, if needed, an amount of sodium hydroxide to bring a pH of the fludarabine phosphate mixture to 6.8; adding, if needed, additional water for injection to achieve the desired total composition volume; stirring the mixture at the desired total composition volume; placing the stirred mixture into one or more storage containers; and sealing the storage containers under nitrogen blanketing. In some embodiments, the method of preparation further comprises sterilizing the stirred mixture before the step of placing the stirred mixture into one or more storage containers; in other embodiments, the method of preparation further comprises sterilizing the stirred mixture after the step of sealing the storage containers under nitrogen blanketing.

In some embodiments, the present disclosure provides a pharmaceutical product suitable for multiple uses (e.g., for injecting multiple doses into a single subject), the pharmaceutical product comprising: a multi-use container; and a sterile composition housed within the multi-use container, the composition including: 25 mg/mL fludarabine phosphate, 0 mg/mL mannitol, 40 mg/mL sorbitol, 1.8 mg/mL methyl paraben, 0.2 mg/mL propyl paraben, 0 mg/mL benzyl alcohol, water for injection, an optional pH adjuster, an inert gaseous atmosphere, and an initial pH level of 6.5 to 7.1. After storage at 5° C.+/−3° C. for at least 12 months, the composition includes: not more than 1.0% iso-ara-guanine-monophosphate; not more than 0.2% isoguanine; not more than 0.2% of any other early-eluting impurity; not more than 0.2% 2-fluoroadenine; not more than 0.2% 2-fluoro-ara-adenine; and not more than 2.0% of any other late-eluting impurity. After storage at 25° C. and 60% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. After storage at 40° C. and 75% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. In some embodiments, the pharmaceutical product is prepared by a method comprising: purging an amount of water for injection representing about 80% of a desired total composition volume with nitrogen gas; heating the purged water for injection to about 70° C.; adding methyl paraben and/or propyl paraben to the heated water for injection under continuous stirring; cooling the mixture of water for injection and methyl paraben and/or propyl paraben to room temperature; adding mannitol and/or sorbitol to the room temperature mixture under continuous stirring; adding fludarabine phosphate to the mixture of methyl paraben and/or propyl paraben, mannitol and/or sorbitol, and water for injection under continuous stirring; adding, if needed, an amount of sodium hydroxide to bring a pH of the fludarabine phosphate mixture to 6.8; adding, if needed, additional water for injection to achieve the desired total composition volume; stirring the mixture at the desired total composition volume; placing the stirred mixture into one or more storage containers; and sealing the storage containers under nitrogen blanketing. In some embodiments, the method of preparation further comprises sterilizing the stirred mixture before the step of placing the stirred mixture into one or more storage containers; in other embodiments, the method of preparation further comprises sterilizing the stirred mixture after the step of sealing the storage containers under nitrogen blanketing.

In some embodiments, the present disclosure provides a pharmaceutical product suitable for multiple uses (e.g., for injecting multiple doses into a single subject), the pharmaceutical product comprising: a multi-use container; and a sterile composition housed within the multi-use container, the composition including: 25 mg/mL fludarabine phosphate, 25 mg/mL mannitol, 0 mg/mL sorbitol, 0 mg/mL methyl paraben, 0 mg/mL propyl paraben, 25 mg/mL benzyl alcohol, water for injection, an optional pH adjuster, an inert gaseous atmosphere, and an initial pH level of 6.5 to 7.1. After storage at 5° C.+/−3° C. for at least 12 months, the composition includes: not more than 1.0% iso-ara-guanine-monophosphate; not more than 0.2% isoguanine; not more than 0.2% of any other early-eluting impurity; not more than 0.2% 2-fluoroadenine; not more than 0.2% 2-fluoro-ara-adenine; and not more than 2.0% of any other late-eluting impurity. After storage at 25° C. and 60% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. After storage at 40° C. and 75% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. In some embodiments, the pharmaceutical product is prepared by a method comprising: purging an amount of water for injection representing about 80% of a desired total composition volume with nitrogen gas; heating the purged water for injection to about 70° C.; adding methyl paraben and/or propyl paraben to the heated water for injection under continuous stirring; cooling the mixture of water for injection and methyl paraben and/or propyl paraben to room temperature; adding mannitol and/or sorbitol to the room temperature mixture under continuous stirring; adding fludarabine phosphate to the mixture of methyl paraben and/or propyl paraben, mannitol and/or sorbitol, and water for injection under continuous stirring; adding, if needed, an amount of sodium hydroxide to bring a pH of the fludarabine phosphate mixture to 6.8; adding, if needed, additional water for injection to achieve the desired total composition volume; stirring the mixture at the desired total composition volume; placing the stirred mixture into one or more storage containers; and sealing the storage containers under nitrogen blanketing. In some embodiments, the method of preparation further comprises sterilizing the stirred mixture before the step of placing the stirred mixture into one or more storage containers; in other embodiments, the method of preparation further comprises sterilizing the stirred mixture after the step of sealing the storage containers under nitrogen blanketing.

In some embodiments, the present disclosure provides a pharmaceutical product suitable for multiple uses (e.g., for injecting multiple doses into a single subject), the pharmaceutical product comprising: a multi-use container; and a sterile composition housed within the multi-use container, the composition including: 30 mg/mL fludarabine phosphate, 40 mg/mL mannitol, 0 mg/mL sorbitol, 0 mg/mL methyl paraben, 0 mg/mL propyl paraben, 0 mg/mL benzyl alcohol, water for injection, an optional pH adjuster, an inert gaseous atmosphere, and an initial pH level of 6.5 to 7.1. After storage at 5° C.+/−3° C. for at least 12 months, the composition includes: not more than 1.0% iso-ara-guanine-monophosphate; not more than 0.2% isoguanine; not more than 0.2% of any other early-eluting impurity; not more than 0.2% 2-fluoroadenine; not more than 0.2% 2-fluoro-ara-adenine; and not more than 2.0% of any other late-eluting impurity. After storage at 25° C. and 60% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. After storage at 40° C. and 75% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. In some embodiments, the pharmaceutical product is prepared by a method comprising: purging an amount of water for injection representing about 80% of a desired total composition volume with nitrogen gas; heating the purged water for injection to about 70° C.; adding methyl paraben and/or propyl paraben to the heated water for injection under continuous stirring; cooling the mixture of water for injection and methyl paraben and/or propyl paraben to room temperature; adding mannitol and/or sorbitol to the room temperature mixture under continuous stirring; adding fludarabine phosphate to the mixture of methyl paraben and/or propyl paraben, mannitol and/or sorbitol, and water for injection under continuous stirring; adding, if needed, an amount of sodium hydroxide to bring a pH of the fludarabine phosphate mixture to 6.8; adding, if needed, additional water for injection to achieve the desired total composition volume; stirring the mixture at the desired total composition volume; placing the stirred mixture into one or more storage containers; and sealing the storage containers under nitrogen blanketing. In some embodiments, the method of preparation further comprises sterilizing the stirred mixture before the step of placing the stirred mixture into one or more storage containers; in other embodiments, the method of preparation further comprises sterilizing the stirred mixture after the step of sealing the storage containers under nitrogen blanketing.

In some embodiments, the present disclosure provides a pharmaceutical product suitable for multiple uses (e.g., for injecting multiple doses into a single subject), the pharmaceutical product comprising: a multi-use container; and a sterile composition housed within the multi-use container, the composition including: 30 mg/mL fludarabine phosphate, 0 mg/mL mannitol, 40 mg/mL sorbitol, 0 mg/mL methyl paraben, 0 mg/mL propyl paraben, 0 mg/mL benzyl alcohol, water for injection, an optional pH adjuster, an inert gaseous atmosphere, and an initial pH level of 6.5 to 7.1. After storage at 5° C.+/−3° C. for at least 12 months, the composition includes: not more than 1.0% iso-ara-guanine-monophosphate; not more than 0.2% isoguanine; not more than 0.2% of any other early-eluting impurity; not more than 0.2% 2-fluoroadenine; not more than 0.2% 2-fluoro-ara-adenine; and not more than 2.0% of any other late-eluting impurity. After storage at 25° C. and 60% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. After storage at 40° C. and 75% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. In some embodiments, the pharmaceutical product is prepared by a method comprising: purging an amount of water for injection representing about 80% of a desired total composition volume with nitrogen gas; heating the purged water for injection to about 70° C.; adding methyl paraben and/or propyl paraben to the heated water for injection under continuous stirring; cooling the mixture of water for injection and methyl paraben and/or propyl paraben to room temperature; adding mannitol and/or sorbitol to the room temperature mixture under continuous stirring; adding fludarabine phosphate to the mixture of methyl paraben and/or propyl paraben, mannitol and/or sorbitol, and water for injection under continuous stirring; adding, if needed, an amount of sodium hydroxide to bring a pH of the fludarabine phosphate mixture to 6.8; adding, if needed, additional water for injection to achieve the desired total composition volume; stirring the mixture at the desired total composition volume; placing the stirred mixture into one or more storage containers; and sealing the storage containers under nitrogen blanketing. In some embodiments, the method of preparation further comprises sterilizing the stirred mixture before the step of placing the stirred mixture into one or more storage containers; in other embodiments, the method of preparation further comprises sterilizing the stirred mixture after the step of sealing the storage containers under nitrogen blanketing.

In some embodiments, the present disclosure provides a pharmaceutical product suitable for multiple uses (e.g., for injecting multiple doses into a single subject), the pharmaceutical product comprising: a multi-use container; and a sterile composition housed within the multi-use container, the composition including: 30 mg/mL fludarabine phosphate, 25 mg/mL mannitol, 0 mg/mL sorbitol, 0 mg/mL methyl paraben, 0 mg/mL propyl paraben, 25 mg/mL benzyl alcohol, water for injection, an optional pH adjuster, an inert gaseous atmosphere, and an initial pH level of 6.5 to 7.1. After storage at 5° C.+/−3° C. for at least 12 months, the composition includes: not more than 1.0% iso-ara-guanine-monophosphate; not more than 0.2% isoguanine; not more than 0.2% of any other early-eluting impurity; not more than 0.2% 2-fluoroadenine; not more than 0.2% 2-fluoro-ara-adenine; and not more than 2.0% of any other late-eluting impurity. After storage at 25° C. and 60% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. After storage at 40° C. and 75% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. In some embodiments, the pharmaceutical product is prepared by a method comprising: purging an amount of water for injection representing about 80% of a desired total composition volume with nitrogen gas; heating the purged water for injection to about 70° C.; adding methyl paraben and/or propyl paraben to the heated water for injection under continuous stirring; cooling the mixture of water for injection and methyl paraben and/or propyl paraben to room temperature; adding mannitol and/or sorbitol to the room temperature mixture under continuous stirring; adding fludarabine phosphate to the mixture of methyl paraben and/or propyl paraben, mannitol and/or sorbitol, and water for injection under continuous stirring; adding, if needed, an amount of sodium hydroxide to bring a pH of the fludarabine phosphate mixture to 6.8; adding, if needed, additional water for injection to achieve the desired total composition volume; stirring the mixture at the desired total composition volume; placing the stirred mixture into one or more storage containers; and sealing the storage containers under nitrogen blanketing. In some embodiments, the method of preparation further comprises sterilizing the stirred mixture before the step of placing the stirred mixture into one or more storage containers; in other embodiments, the method of preparation further comprises sterilizing the stirred mixture after the step of sealing the storage containers under nitrogen blanketing.

In some embodiments, the present disclosure provides a pharmaceutical product suitable for multiple uses (e.g., for injecting multiple doses into a single subject), the pharmaceutical product comprising: a multi-use container; and a sterile composition housed within the multi-use container, the composition including: 30 mg/mL fludarabine phosphate, 25 mg/mL mannitol, 0 mg/mL sorbitol, 1.8 mg/mL methyl paraben, 0.2 mg/mL propyl paraben, 0 mg/mL benzyl alcohol, water for injection, an optional pH adjuster, an inert gaseous atmosphere, and an initial pH level of 6.5 to 7.1. After storage at 5° C.+/−3° C. for at least 12 months, the composition includes: not more than 1.0% iso-ara-guanine-monophosphate; not more than 0.2% isoguanine; not more than 0.2% of any other early-eluting impurity; not more than 0.2% 2-fluoroadenine; not more than 0.2% 2-fluoro-ara-adenine; and not more than 2.0% of any other late-eluting impurity. After storage at 25° C. and 60% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. After storage at 40° C. and 75% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. In some embodiments, the pharmaceutical product is prepared by a method comprising: purging an amount of water for injection representing about 80% of a desired total composition volume with nitrogen gas; heating the purged water for injection to about 70° C.; adding methyl paraben and/or propyl paraben to the heated water for injection under continuous stirring; cooling the mixture of water for injection and methyl paraben and/or propyl paraben to room temperature; adding mannitol and/or sorbitol to the room temperature mixture under continuous stirring; adding fludarabine phosphate to the mixture of methyl paraben and/or propyl paraben, mannitol and/or sorbitol, and water for injection under continuous stirring; adding, if needed, an amount of sodium hydroxide to bring a pH of the fludarabine phosphate mixture to 6.8; adding, if needed, additional water for injection to achieve the desired total composition volume; stirring the mixture at the desired total composition volume; placing the stirred mixture into one or more storage containers; and sealing the storage containers under nitrogen blanketing. In some embodiments, the method of preparation further comprises sterilizing the stirred mixture before the step of placing the stirred mixture into one or more storage containers; in other embodiments, the method of preparation further comprises sterilizing the stirred mixture after the step of sealing the storage containers under nitrogen blanketing.

In some embodiments, the present disclosure provides a pharmaceutical product suitable for multiple uses (e.g., for injecting multiple doses into a single subject), the pharmaceutical product comprising: a multi-use container; and a sterile composition housed within the multi-use container, the composition including: 30 mg/mL fludarabine phosphate, 0 mg/mL mannitol, 40 mg/mL sorbitol, 1.8 mg/mL methyl paraben, 0.2 mg/mL propyl paraben, 0 mg/mL benzyl alcohol, water for injection, an optional pH adjuster, an inert gaseous atmosphere, and an initial pH level of 6.5 to 7.1. After storage at 5° C.+/−3° C. for at least 12 months, the composition includes: not more than 1.0% iso-ara-guanine-monophosphate; not more than 0.2% isoguanine; not more than 0.2% of any other early-eluting impurity; not more than 0.2% 2-fluoroadenine; not more than 0.2% 2-fluoro-ara-adenine; and not more than 2.0% of any other late-eluting impurity. After storage at 25° C. and 60% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. After storage at 40° C. and 75% relative humidity for 2 weeks, the composition includes: not more than 1.0% iso-ara-guanine-monophosphate; not more than 0.2% isoguanine; not more than 0.2% of a 3,5 diphosphate analog; not more than 0.2% of any other early-eluting impurity; not more than 0.2% 2-fluoroadenine; not more than 0.2% 2-fluoro-ara-adenine; not more than 1.0% of a 2-ethoxyphosphate analog; and not more than 2.0% of any other late-eluting impurity. In some embodiments, the pharmaceutical product is prepared by a method comprising: purging an amount of water for injection representing about 80% of a desired total composition volume with nitrogen gas; heating the purged water for injection to about 70° C.; adding methyl paraben and/or propyl paraben to the heated water for injection under continuous stirring; cooling the mixture of water for injection and methyl paraben and/or propyl paraben to room temperature; adding mannitol and/or sorbitol to the room temperature mixture under continuous stirring; adding fludarabine phosphate to the mixture of methyl paraben and/or propyl paraben, mannitol and/or sorbitol, and water for injection under continuous stirring; adding, if needed, an amount of sodium hydroxide to bring a pH of the fludarabine phosphate mixture to 6.8; adding, if needed, additional water for injection to achieve the desired total composition volume; stirring the mixture at the desired total composition volume; placing the stirred mixture into one or more storage containers; and sealing the storage containers under nitrogen blanketing. In some embodiments, the method of preparation further comprises sterilizing the stirred mixture before the step of placing the stirred mixture into one or more storage containers; in other embodiments, the method of preparation further comprises sterilizing the stirred mixture after the step of sealing the storage containers under nitrogen blanketing.

### Methods of Using Stable Fludarabine Compositions

The present disclosure provides methods of using ready-to-use compositions comprising fludarabine and/or fludarabine phosphate. Generally, the methods disclosed herein do not require a clinician to dilute or reconstitute the sterile, packaged ready-to-use fludarabine composition before administering the fludarabine composition to a subject. In some embodiments, the risk of microbial contamination to the subject is lower, such as substantially lower, when administered a ready-to-use fludarabine composition consistent with the present disclosure compared to the risk of microbial contamination to subjects (e.g., subjects having the same or similar characteristics and/or diagnosis) who are administered fludarabine compositions that require dilution or reconstitution before administration.

In some embodiments, the present disclosure provides a method of reducing a risk of microbial infection associated with administration of fludarabine or fludarabine phosphate to a subject in need thereof. In some embodiments, the subject has been diagnosed with a lymphoma. In some embodiments, the subject has been diagnosed with a leukemia. In some embodiments, the subject has been diagnosed with chronic lymphocytic leukemia (CLL). In some embodiments, the subject is associated with a therapeutic regimen comprising CAR-T therapy. In some embodiments, the subject is associated with a therapeutic regimen comprising lymphodepletion.

In some embodiments, a risk of microbial contamination to a subject administered a ready-to-use composition comprising fludarabine (e.g., fludarabine and/or fludarabine phosphate) consistent with the present disclosure is lower, or substantially lower, than a risk of microbial contamination to a second subject administered fludarabine (e.g., fludarabine and/or fludarabine phosphate) that has been diluted or reconstituted (e.g., diluted or reconstituted by a clinician) before administration to the second subject. In some such embodiments, the second subject receives fludarabine (e.g., fludarabine and/or fludarabine phosphate) prepared by diluting an aliquot of a sterilized packaged pharmaceutical product comprising 30 mg/mL fludarabine phosphate with a diluent, such as water, 5% dextrose, or saline, to form the diluted fludarabine composition. In other embodiments, the second subject receives fludarabine (e.g., fludarabine and/or fludarabine phosphate) prepared by reconstituting a lyophilized fludarabine phosphate composition with water, 5% dextrose, or saline.

### Lymphodepletion Methods

In some embodiments, the present disclosure provides a method of lymphodepleting a subject in need thereof, the method comprising administering to the subject an amount of fludarabine not exceeding about 25 mg/m². In some embodiments, the fludarabine comprises, consists essentially of, or consists of fludarabine phosphate. In some embodiments, the step of lymphodepleting comprises withdrawing a sterile premixed solution comprising not more than about 25 mg/mL fludarabine phosphate from a container into a syringe; and injecting the solution into the subject to provide an amount of fludarabine phosphate not exceeding about 25 mg/m². In some embodiments, the method does not include mixing contents of the container with water, 5% dextrose, or saline before the step of withdrawing the sterile premixed solution into the syringe. In some embodiments, the sterile premixed solution is prepared by a process comprising: mixing fludarabine phosphate, mannitol, an optional pH adjuster and water to form a bulk solution having an initial pH level of about 6.5 to about 7.1; filtering the bulk solution through a sterilization grade filter to form a sterile bulk solution; filling one or more containers with at least a portion of the sterile bulk solution; and sealing the one or more containers. In some embodiments, the sterilization grade filter has a pore size of about 0.2 microns. In some embodiments, the process further includes sterilizing the one or more containers (e.g., by autoclave) before the step of filling the one or more containers. In some embodiments, the sterile premixed solution comprises: not more than 25 mg/mL fludarabine phosphate; a tonicity agent (e.g., mannitol or sorbitol); water; and an initial pH level of about 6.5 to about 7.1. In some embodiments, the sterile premixed solution consists essentially of: not more than 25 mg/mL fludarabine phosphate; a tonicity agent (e.g., mannitol); water; and an initial pH level of about 6.5 to about 7.1. In some embodiments, the sterile premixed solution consists of: not more than 25 mg/mL fludarabine phosphate; a tonicity agent (e.g., mannitol); water; and an initial pH level of about 6.5 to about 7.1. In some embodiments, the sterile premixed solution has an initial pH level of about 6.6 to about 7.0. In some embodiments, the sterile premixed solution has an initial pH level of about 6.8. In some embodiments, the sterile premixed solution has after storage at about 5° C. for 3 months, a pH level within 0.5 pH units of the initial pH level. In some embodiments, the sterile premixed solution has, after storage at about 5° C. for 6 months, a pH level within 0.5 pH units of the initial pH level. In some embodiments, the sterile premixed solution has, after storage at about 5° C. for 9 months, a pH level within 0.5 pH units of the initial pH level. In some embodiments, the sterile premixed solution has, after storage at about 5° C. for 18 months, a pH level within 0.5 pH units of the initial pH level. In some embodiments, the sterile premixed solution has, after storage at about 5° C. for 24 months, a pH level within 0.5 pH units of the initial pH level. In some embodiments, the sterile premixed solution has, after storage at about 25° C. and about 60% relative humidity (RH) for 1 month, a pH level within 0.5 pH units of the initial pH level. In some embodiments, the sterile premixed solution has, after storage at about 25° C. and about 60% relative humidity (RH) for 2 months, a pH level within 0.5 pH units of the initial pH level. In some embodiments, the sterile premixed solution has, after storage at about 25° C. and about 60% relative humidity (RH) for 3 months, a pH level within 0.5 pH units of the initial pH level. In some embodiments, the step of lymphodepleting further comprises administering cyclophosphamide to the subject. In some embodiments, the cyclophosphamide is administered in an amount less than 300 mg/m². In some embodiments, the cyclophosphamide is administered in an amount of about 300 mg/m².

In some embodiments, the method comprises administering to the subject an amount of fludarabine (e.g., fludarabine and/or fludarabine phosphate) not exceeding about 25 mg/m². In some embodiments, the method comprises administering to the subject an amount of fludarabine (e.g., fludarabine and/or fludarabine phosphate) not exceeding about 24 mg/m². In some embodiments, the method comprises administering to the subject an amount of fludarabine (e.g., fludarabine and/or fludarabine phosphate) not exceeding about 23 mg/m². In some embodiments, the method comprises administering to the subject an amount of fludarabine (e.g., fludarabine and/or fludarabine phosphate) not exceeding about 22 mg/m². In some embodiments, the method comprises administering to the subject an amount of fludarabine (e.g., fludarabine and/or fludarabine phosphate) not exceeding about 21 mg/m². In some embodiments, the method comprises administering to the subject an amount of fludarabine (e.g., fludarabine and/or fludarabine phosphate) not exceeding about 20 mg/m². In some embodiments, the method comprises administering to the subject an amount of fludarabine (e.g., fludarabine and/or fludarabine phosphate) not exceeding about 19 mg/m². In some embodiments, the method comprises administering to the subject an amount of fludarabine (e.g., fludarabine and/or fludarabine phosphate) not exceeding about 18 mg/m². In some embodiments, the method comprises administering to the subject an amount of fludarabine (e.g., fludarabine and/or fludarabine phosphate) not exceeding about 17 mg/m². In some embodiments, the method comprises administering to the subject an amount of fludarabine (e.g., fludarabine and/or fludarabine phosphate) not exceeding about 16 mg/m². In some embodiments, the method comprises administering to the subject an amount of fludarabine (e.g., fludarabine and/or fludarabine phosphate) not exceeding about 15 mg/m². In some embodiments, the method comprises administering to the subject an amount of fludarabine (e.g., fludarabine and/or fludarabine phosphate) not exceeding about 14 mg/m². In some embodiments, the method comprises administering to the subject an amount of fludarabine (e.g., fludarabine and/or fludarabine phosphate) not exceeding about 13 mg/m². In some embodiments, the method comprises administering to the subject an amount of fludarabine (e.g., fludarabine and/or fludarabine phosphate) not exceeding about 12 mg/m². In some embodiments, the method comprises administering to the subject an amount of fludarabine (e.g., fludarabine and/or fludarabine phosphate) not exceeding about 11 mg/m². In some embodiments, the method comprises administering to the subject an amount of fludarabine (e.g., fludarabine and/or fludarabine phosphate) not exceeding about 10 mg/m². In some embodiments, the method comprises administering to the subject an amount of fludarabine (e.g., fludarabine and/or fludarabine phosphate) not exceeding about 9 mg/m². In some embodiments, the method comprises administering to the subject an amount of fludarabine (e.g., fludarabine and/or fludarabine phosphate) not exceeding about 8 mg/m². In some embodiments, the method comprises administering to the subject an amount of fludarabine (e.g., fludarabine and/or fludarabine phosphate) not exceeding about 7 mg/m². In some embodiments, the method comprises administering to the subject an amount of fludarabine (e.g., fludarabine and/or fludarabine phosphate) not exceeding about 6 mg/m². In some embodiments, the method comprises administering to the subject an amount of fludarabine (e.g., fludarabine and/or fludarabine phosphate) not exceeding about 5 mg/m².

In some embodiments, a subject lymphodepleted by a method consistent with the present disclosure (e.g., comprising administering fludarabine and/or fludarabine phosphate to the subject in an amount not exceeding about 25 mg/m²) exhibits fewer side effects and/or less severe side effects than a comparable subject lymphodepleted by a similar method comprising administering fludarabine and/or fludarabine phosphate to the comparable subject in an amount of about 30 mg/m². In some embodiments, the side effect is one or more of: arm pain; back pain; jaw pain; black, tarry stools; blood in the urine or stools; chest pain or discomfort; chest tightness or heaviness; constipation; cough or hoarseness; coughing or spitting up blood; fast or irregular heartbeat; fever or chills; general feeling of discomfort or illness; lower back or side pain; nausea; pain; painful, burning, or difficult urination; pale skin; pinpoint red spots on the skin; shortness of breath; sneezing; sore throat; sores, ulcers, or white spots on the lips or in the mouth; stomach pain, severe; sweating; swelling; tender, swollen glands in the neck; thickening of bronchial secretions; troubled breathing; unusual bleeding or bruising; unusual tiredness or weakness; vomiting of blood or material that looks like coffee grounds; and wheezing; agitation; aneurysm; bleeding gums; blurred vision; confusion; decreased urine output; difficulty in breathing or swallowing; dilated neck veins; dizziness; extreme fatigue; fainting; fast, pounding, or irregular heartbeat or pulse; headache; increased menstrual flow or vaginal bleeding; irregular breathing; loss of hearing; nosebleeds; numbness or tingling in the fingers, toes, or face; pain, redness, or swelling in the arm or leg; paralysis; prolonged bleeding from cuts; seizures; slurred speech; sudden and severe inability to speak; temporary blindness; weakness in the arm or leg on one side of the body, sudden and severe; weight gain; blindness; continuing vomiting; dark-colored urine; drowsiness; frequent urination; hives; itching; light-colored stools; loss of appetite; loss of consciousness; lower abdominal cramping; muscle tremors; puffiness or swelling of the eyelids or around the eyes, face, lips, or tongue; rapid, deep breathing; restlessness; skin rash; stomach pain; trouble speaking, thinking, or walking; yellow eyes or skin; abdominal pain; bladder pain; body aches or pain; burning, crawling, itching, numbness, prickling, “pins and needles”, or tingling feelings; cloudy urine; congestion; diarrhea; difficulty in moving; dry mouth or throat; flushed, dry skin; frequent urge to urinate; fruit-like breath odor; increased hunger; increased thirst; increased urination; joint pain; muscle aching or cramping; muscle pains or stiffness; runny nose; swollen joints; trouble in swallowing; voice changes; weight loss; abdominal fullness; bluish color of skin; changes in skin color; cracked lips; dandruff; decrease in height; decreased urination; difficulty in sleeping; discouragement; feeling sad or empty; gaseous abdominal pain; heartburn; irritability; loss of interest or pleasure; lightheadedness; oily skin; pain or tenderness around the eyes and cheekbones; rapid breathing; recurrent fever; stuffy nose; sunken eye; trouble concentrating; and/or wrinkled skin.

In some embodiments, the subject is associated with a therapeutic regimen comprising CAR-T therapy. In some embodiments, the subject has been diagnosed with a lymphoma. In some embodiments, the subject has been diagnosed with a leukemia.

In any embodiment disclosed herein, the lymphodepletion method does not require administration (e.g., co-administration) of cyclophosphamide.

In any embodiment disclosed herein, the lymphodepletion method does not require administration (e.g., prior administration or co-administration) of a protease inhibitor, an immunomodulatory drug, and/or an anti-CD-38 antibody.

### Methods of Treating Lymphomas and Leukemias

In some embodiments, the present disclosure provides methods of treating a lymphoma or a leukemia in a subject in need thereof, the method comprising: lymphodepleting the subject by administering an amount of fludarabine not exceeding about 25 mg/m²; and thereafter administering CAR-T cells to the subject. In some embodiments, the method further comprises collecting T cells from the subject before the step of administering the fludarabine to the subject. In some embodiments, the fludarabine comprises, consists essentially of, or consists of fludarabine phosphate. In some embodiments, the step of lymphodepleting comprises: withdrawing a sterile premixed solution comprising not more than about 25 mg/mL fludarabine phosphate from a container into a syringe; and injecting the solution into the subject to provide an amount of fludarabine phosphate not exceeding about 25 mg/m². In some embodiments, the method does not include mixing contents of the container with water before the step of withdrawing the sterile premixed solution into the syringe. In some embodiments, the sterile premixed solution is prepared by a process comprising: mixing fludarabine phosphate, mannitol, an optional pH adjuster and water to form a bulk solution having an initial pH level of about 6.5 to about 7.1; filtering the bulk solution through a sterilization grade filter to form a sterile bulk solution; filling one or more containers with at least a portion of the sterile bulk solution; and sealing the one or more containers. In some embodiments, the sterilization grade filter has a pore size of about 0.2 microns. In some embodiments, the process of preparing the sterile premixed solution further comprises sterilizing the one or more containers before the step of filling the one or more containers. In some embodiments, the sterile premixed solution comprises: not more than 25 mg/mL fludarabine phosphate; a tonicity agent; water; and an initial pH level of about 6.5 to about 7.1. In some embodiments, the sterile premixed solution consists essentially of: not more than 25 mg/mL fludarabine phosphate; a tonicity agent; water; and an initial pH level of about 6.5 to about 7.1. In some embodiments, the sterile premixed solution consists of: not more than 25 mg/mL fludarabine phosphate; a tonicity agent; water; and an initial pH level of about 6.5 to about 7.1. In some embodiments, the sterile premixed solution has an initial pH level of about 6.6 to about 7.0. In some embodiments, the sterile premixed solution has an initial pH level of about 6.8. In some embodiments, the sterile premixed solution has, after storage at about 5° C. for 3 months, a pH level within 0.5 pH units of the initial pH level. In some embodiments, the sterile premixed solution has, after storage at about 5° C. for 6 months, a pH level within 0.5 pH units of the initial pH level. In some embodiments, the sterile premixed solution has, after storage at about 5° C. for 9 months, a pH level within 0.5 pH units of the initial pH level. In some embodiments, the sterile premixed solution has, after storage at about 5° C. for 18 months, a pH level within 0.5 pH units of the initial pH level. In some embodiments, the sterile premixed solution has, after storage at about 5° C. for 24 months, a pH level within 0.5 pH units of the initial pH level. In some embodiments, the sterile premixed solution has, after storage at about 25° C. and about 60% relative humidity (RH) for 1 month, a pH level within 0.5 pH units of the initial pH level. In some embodiments, the sterile premixed solution has, after storage at about 25° C. and about 60% relative humidity (RH) for 2 months, a pH level within 0.5 pH units of the initial pH level. In some embodiments, the sterile premixed solution has, after storage at about 25° C. and about 60% relative humidity (RH) for 3 months, a pH level within 0.5 pH units of the initial pH level. In some embodiments, the step of administering the sterile premixed solution comprises injecting the solution through skin of the subject. In some embodiments, the step of lymphodepleting further comprises administering cyclophosphamide to the subject. In some embodiments, the cyclophosphamide and the fludarabine are co-administered to the subject. In some embodiments, the cyclophosphamide is administered in an amount less than 300 mg/m². In some embodiments, the cyclophosphamide is administered in an amount of about 300 mg/m².

In some embodiments, the subject is associated with a therapeutic regimen comprising CAR-T therapy. In some embodiments, the subject has been diagnosed with a lymphoma. In some embodiments, the subject has been diagnosed with a leukemia.

In any embodiment disclosed herein, the method of treating a lymphoma or a leukemia does not require administration (e.g., co-administration) of cyclophosphamide.

In any embodiment disclosed herein, the method of treating a lymphoma or a leukemia does not require administration (e.g., prior administration or co-administration) of a protease inhibitor, an immunomodulatory drug, and/or an anti-CD-38 antibody.

### EXAMPLES

Aspects of embodiments may be further understood in light of the following examples, which should not be construed as limiting in any way.

### Example 1. Ready-to-Use Fludarabine Phosphate Composition in Single-Use Vials

A 10.75-L batch of fludarabine phosphate composition consistent with the present disclosure was prepared by combining 268.75 g of mannitol and water for injection (q.s. to 10.75 L) under positive nitrogen pressure. Fludarabine phosphate (268.75 g) was then added under positive nitrogen pressure and mixed thoroughly. The pH level of the bulk solution was adjusted with 1 N sodium hydroxide to provide an initial pH level of 6.8+/−0.2. The pH-adjusted bulk solution satisfied the quality parameters listed in Table 1, below:

TABLE 1
In-Process Analytical and Microbiological Testing

| | |
|---|---|
| Test | Alert or Response Limit |
| Appearance | Clear (not more than 3.0 NTU) and colorless |
| | solution (color shall not be more intense than B9) |
| Density | 0.9644-1.0658 g/cm³ |
| pH | 6.5-7.1 |
| Assay | It contains the equivalent of not less than 95.00% |
| | and not more than 105.00% of the labeled |
| | amount of Fludarabine Phosphate, USP |
| Bioburden | Alert limit: 5 CFU/100 mL |
| | Action limit: 10 CFU/100 mL |

The pH-adjusted bulk solution was then filtered through a 0.2-micron filter (Ultipor® N66 Sterilizing-Grade Filter Cartridges, Pall Corporation).

Type I glass vials were washed, dried, and sterilized by autoclave. Bromobutyl rubber stoppers were sterilized by autoclave and dried. Aluminum flip caps were sterilized by autoclave and dried. 2-mL aliquots of the sterile filtered bulk solution were placed in the sterilized glass vials, which were then stopped with the bromobutyl rubber stoppers and sealed with the aluminum flip caps, all under nitrogen pressure. The sealed vials were stored at 2° C. to 8° C.

The final stored composition satisfied the quality parameters listed in Table 2, and had the characteristics listed in Table 3, below:

TABLE 2
Drug Product Specifications

| Test | Release | | Stability | | Method |
|---|---|---|---|---|---|
| Description | Clear, colorless solution in | | Clear, colorless solution in | | Visual |
| | colorless glass vial stoppered | | colorless glass vial stoppered | | |
| | and sealed with aluminum | | and sealed with aluminum | | |
| | flip-off cap | | flip-off cap | | |
| Identification | Conforms to standard UV | | N/A | | USP |
| (UV) | spectrum | | | | <197U> |
| Identification | The retention time of the major | | N/A | | USP |
| (HPLC) | peak in the chromatogram of | | | | monograph |
| | the test solution corresponds | | | | |
| | to that in the chromatogram | | | | |
| | of the standard solution, as | | | | |
| | obtained in the assay | | | | |
| Appearance | Colorless (color is not more | | Colorless (color is not more | | EP <2.2.1, |
| of solution | intense than standard B9) and | | intense than standard B9) and | | 2.2.2> |
| | clear (NMT 3.0 NTU) solution | | clear (NMT 3.0 NTU) solution | | |
| pH | Between 6.5-7.1 (at 25 ± 2° C.) | | Between 6.0-7.1 (at 25 ± 2° C.) | | USP <791> |
| Volume in | NLT labeled size (2.0 mL) | | N/A | | USP <1> |
| Container | | | | | |
| Assay | NLT 95.0% and NMT 105.0% | | NLT 95.0% and NMT 105.0% | | USP |
| | of the labeled amounts of | | of the labeled amounts of | | monograph |
| | Fludarabine Phosphate. | | Fludarabine Phosphate. | | |
| Related | Procedure 1 (Early-eluting | | Procedure 1 (Early-eluting | | USP |
| Compounds | impurities) | | impurities) | | Monograph |
| (organic | Iso-ara-guanine- | NMT 0.8% | Iso-ara-guanine- | NMT 1.0% | |
| Impurities) | monophosphate: | | monophosphate: | | |
| | Isoguanine: | NMT 0.2% | Isoguanine: | NMT 0.2% | |
| | Any other | NMT 0.2% | Any other | NMT 0.2% | |
| | impurity: | | impurity: | | |
| | Procedure 2 (Late-eluting | | Procedure 2 (Late-eluting | | |
| | impurities) | | impurities) | | |
| | 2-Fluoroadenine: | NMT 0.1% | 2-Fluoroadenine: | NMT 0.2% | |
| | 2-Fluoro-ara- | NMT 0.1% | 2-Fluoro-ara- | NMT 0.2% | |
| | adenine: | | adenine: | | |
| | Any other | NMT 0.2% | Any other | NMT 0.2% | |
| | impurities: | | impurities: | | |
| | Total Impurities | NMT 1.5% | Total Impurities | NMT 2.0% | |
| | (Procedure 1 + | | (Procedure 1 + | | |
| | Procedure 2) | | Procedure 2) | | |
| Residual | Complies | | Complies | | USP <467> |
| Solvents | | | | | |

| | | | |
|---|---|---|---|
| Particulate | Subvisible particles ≥10 μm | Subvisible particles ≥10 μm | USP <788> |
| Matter | particles: NMT 6000/vial ≥25 | particles: NMT 6000/vial ≥25 | |
| | μm particles: NMT 600/vial | μm particles: NMT 600/vial | |
| Sterility | Sterile | Sterile | USP <71> |
| Antimicrobial | Complies | Complies | USP <51> |
| Effectiveness | | | |
| Bacterial | NMT 7.7 USP EU/mg of | NMT 7.7 USP EU/mg of | USP <85> |
| Endotoxins | Fludarabine Phosphate | Fludarabine Phosphate | |

TABLE 3
Formula Card.

| | | Storage | Amount per | Amount |
|---|---|---|---|---|
| Component | Function | conditions | 10.75-L batch | per mL |
| Fludarabine | Active agent | 2-8° C. | 268.75 g | 25 mg |
| phosphate, USP | | | | |
| Mannitol, USP | Tonicity agent | 15-30° C. | 268.75 g | 25 mg |
| Sodium | pH adjuster | 15-30° C. | To adjust pH to 6.8 | |
| hydroxide, USP | | | | |
| Water for | Solvent | N/A | q.s. to | q.s. to |
| injection | | | 10.75 L | 1 mL |
| Nitrogen NF | Inert | N/A | Inert atmosphere | |
| | atmosphere | | | |

### Example 2. Ready-to-Use Fludarabine Phosphate Composition in Single-Use Vials

A 10.75-L batch of fludarabine phosphate composition consistent with the present disclosure is prepared by combining 268.75 g of sorbitol and water for injection (q.s. to 10.75 L) under positive nitrogen pressure. Fludarabine phosphate (268.75 g) is then added under positive nitrogen pressure and mixed thoroughly. The pH level of the bulk solution is adjusted with 1 N sodium hydroxide to provide an initial pH level of 6.8+/−0.2. The pH-adjusted bulk solution satisfies the quality parameters listed in Table 1, above.

The pH-adjusted bulk solution is then filtered through a 0.2-micron filter e.g., (Ultipor® N66 Sterilizing-Grade Filter Cartridges, Pall Corporation).

Type I glass vials are washed, dried, and sterilized by autoclave. Bromobutyl rubber stoppers are sterilized by autoclave and dried. Aluminum flip caps are sterilized by autoclave and dried. 5-mL aliquots of the sterile filtered bulk solution are placed in the sterilized glass vials, which are then stopped with the bromobutyl rubber stoppers and sealed with the aluminum flip caps, all under nitrogen pressure. The sealed vials are stored at 2° C. to 8° C.

The final stored composition satisfies the quality parameters listed in Table 2, above, and has the characteristics shown in Table 4, below:

TABLE 4
Formula Card.

| | | Storage | Amount per | |
|---|---|---|---|---|
| Component | Function | conditions | 10.75-L batch | Amount per mL |
| Fludarabine phosphate, USP | Active agent | 2-8° C. | 268.75 g | 25 mg |
| Sorbitol, USP | Tonicity agent | 15-30° C. | 268.75 g | 25 mg |
| Sodium hydroxide, USP | pH adjuster | 15-30° C. | To adjust pH to 6.8 | |
| Water for injection | Solvent | N/A | q.s. to 10.75 L | q.s. to 1 mL |

| | | | |
|---|---|---|---|
| Nitrogen NF | Inert atmosphere | N/A | Inert atmosphere |

### Example 3. Ready-to-Use Fludarabine Phosphate Composition in Multi-Dose Vials

A 25-L batch of fludarabine phosphate composition consistent with the present disclosure is prepared by combining mannitol (1000 g), methyl paraben (62.5 g), and water for injection (q.s. to 25 L) under positive nitrogen pressure. Fludarabine phosphate (750 g) is then added under positive nitrogen pressure and mixed thoroughly. The pH level of the bulk solution is adjusted with 1 N sodium hydroxide to provide an initial pH level of 6.8+/−0.2. The pH-adjusted bulk solution satisfies the quality parameters listed in Table 1, above.

The pH-adjusted bulk solution is then filtered through a 0.2-micron filter (e.g., Ultipor® N66 Sterilizing-Grade Filter Cartridges, Pall Corporation).

Type I glass vials are washed, dried, and sterilized by autoclave. Bromobutyl rubber stoppers are sterilized by autoclave and dried. Aluminum flip caps are sterilized by autoclave and dried. 5-mL aliquots of the sterile filtered bulk solution are placed in the sterilized glass vials, which are then stopped with the bromobutyl rubber stoppers and sealed with the aluminum flip caps, all under nitrogen pressure. The sealed vials are stored at 2° C. to 8° C.

The final stored composition satisfies the quality parameters listed in Table 2, above, and has the characteristics shown in Table 5, below:

TABLE 5
Formula Card.

| | | Storage | Amount per | | | |
|---|---|---|---|---|---|---|
| Component | Function | conditions | 25-L batch | | Amount per mL | |
| Fludarabine phosphate, USP | Active agent | 2-8° C. | 750 | g | 30 | mg |
| Mannitol, USP | Tonicity agent | 15-30° C. | 1000 | g | 40 | mg |
| Methyl paraben | Preservative | 15-30° C. | 62.5 | g | 2.5 | mg |

| Sodium hydroxide, USP | pH adjuster | 15-30° C. | To adjust pH to 6.8 | |
|---|---|---|---|---|
| Water for injection | Solvent | N/A | q.s. to 25 L | q.s. to 1 mL |

| | | | |
|---|---|---|---|
| Nitrogen NF | Inert atmosphere | N/A | Inert atmosphere |

### Example 4. Ready-to-Use Fludarabine Phosphate Composition in Multi-Dose Vials

A 25-L batch of fludarabine phosphate composition consistent with the present disclosure is prepared by combining sorbitol (1000 g), methyl paraben (62.5 g), and water for injection (q.s. to 25 L) under positive nitrogen pressure. Fludarabine phosphate (750 g) is then added under positive nitrogen pressure and mixed thoroughly. The pH level of the bulk solution is adjusted with 1 N sodium hydroxide to provide an initial pH level of 6.8+/−0.2. The pH-adjusted bulk solution satisfies the quality parameters listed in Table 1, above.

The pH-adjusted bulk solution is then filtered through a 0.2-micron filter (e.g., Ultipor® N66 Sterilizing-Grade Filter Cartridges, Pall Corporation).

Type I glass vials are washed, dried, and sterilized by autoclave. Bromobutyl rubber stoppers are sterilized by autoclave and dried. Aluminum flip caps are sterilized by autoclave and dried. 5-mL aliquots of the sterile filtered bulk solution are placed in the sterilized glass vials, which are then stopped with the bromobutyl rubber stoppers and sealed with the aluminum flip caps, all under nitrogen pressure. The sealed vials are stored at 2° C. to 8° C.

The final stored composition satisfies the quality parameters listed in Table 2, above, and has the characteristics shown in Table 6, below:

TABLE 6
Formula Card.

| | | Storage | Amount per | | | |
|---|---|---|---|---|---|---|
| Component | Function | conditions | 25-L batch | | Amount per mL | |
| Fludarabine phosphate, USP | Active agent | 2-8° C. | 750 | g | 30 | mg |
| Sorbitol, USP | Tonicity agent | 15-30° C. | 1000 | g | 40 | mg |
| Methyl paraben | Preservative | 15-30° C. | 62.5 | g | 2.5 | mg |

| Sodium hydroxide, USP | pH adjuster | 15-30° C. | To adjust pH to 6.8 | |
|---|---|---|---|---|
| Water for injection | Solvent | N/A | q.s. to 25 L | q.s. to 1 mL |

| | | | |
|---|---|---|---|
| Nitrogen NF | Inert atmosphere | N/A | Inert atmosphere |

### Example 5. Preservative-Free Ready-to-Use Fludarabine Phosphate Composition in Multi-Dose Vials

A 25-L batch of preservative-free fludarabine phosphate composition consistent with the present disclosure is prepared by combining mannitol (1000 g), and water for injection (q.s. to 25 L) under positive nitrogen pressure. Fludarabine phosphate (750 g) is then added under positive nitrogen pressure and mixed thoroughly. The pH level of the bulk solution is adjusted with 1 N sodium hydroxide to provide an initial pH level of 6.8+/−0.2. The pH-adjusted bulk solution satisfies the quality parameters listed in Table 1, above.

The pH-adjusted bulk solution is then filtered through a 0.2-micron filter (e.g., Ultipor® N66 Sterilizing-Grade Filter Cartridges, Pall Corporation).

Type I glass vials are washed, dried, and sterilized by autoclave. Bromobutyl rubber stoppers are sterilized by autoclave and dried. Aluminum flip caps are sterilized by autoclave and dried. 5-mL aliquots of the sterile filtered bulk solution are placed in the sterilized glass vials, which are then stopped with the bromobutyl rubber stoppers and sealed with the aluminum flip caps, all under nitrogen pressure. The sealed vials are stored at 2° C. to 8° C.

The final stored composition satisfies the quality parameters listed in Table 2, above, and has the characteristics shown in Table 7, below:

TABLE 7
Formula Card.

| | | Storage | Amount per | |
|---|---|---|---|---|
| Component | Function | conditions | 25-L batch | Amount per mL |
| Fludarabine phosphate, USP | Active agent | 2-8° C. | 750 g | 30 mg |
| Mannitol, USP | Tonicity agent | 15-30° C. | 1000 g | 40 mg |
| Sodium hydroxide, USP | pH adjuster | 15-30° C. | To adjust pH to 6.8 | |
| Water for injection | Solvent | N/A | q.s. to 25 L | q.s. to 1 mL |

| | | | |
|---|---|---|---|
| Nitrogen NF | Inert atmosphere | N/A | Inert atmosphere |

### Example 6. Preservative-Free Ready-to-Use Fludarabine Phosphate Composition in Multi-Dose Vials

A 25-L batch of preservative-free fludarabine phosphate composition consistent with the present disclosure is prepared by combining sorbitol (1000 g), and water for injection (q.s. to 25 L) under positive nitrogen pressure. Fludarabine phosphate (750 g) is then added under positive nitrogen pressure and mixed thoroughly. The pH level of the bulk solution is adjusted with 1 N sodium hydroxide to provide an initial pH level of 6.8+/−0.2. The pH-adjusted bulk solution satisfies the quality parameters listed in Table 1, above.

The pH-adjusted bulk solution is then filtered through a 0.2-micron filter (e.g., Ultipor® N66 Sterilizing-Grade Filter Cartridges, Pall Corporation).

Type I glass vials are washed, dried, and sterilized by autoclave. Bromobutyl rubber stoppers are sterilized by autoclave and dried. Aluminum flip caps are sterilized by autoclave and dried. 5-mL aliquots of the sterile filtered bulk solution are placed in the sterilized glass vials, which are then stopped with the bromobutyl rubber stoppers and sealed with the aluminum flip caps, all under nitrogen pressure. The sealed vials are stored at 2° C. to 8° C.

The final stored composition satisfies the quality parameters listed in Table 2, above, and has the characteristics listed in Table 8, below:

TABLE 8
Formula Card.

| | | Storage | Amount per | |
|---|---|---|---|---|
| Component | Function | conditions | 25-L batch | Amount per mL |
| Fludarabine phosphate, USP | Active agent | 2-8° C. | 750 g | 30 mg |
| Sorbitol, USP | Tonicity agent | 15-30° C. | 1000 g | 40 mg |
| Sodium hydroxide, USP | pH adjuster | 15-30° C. | To adjust pH to 6.8 | |
| Water for injection | Solvent | N/A | q.s. to 25 L | q.s. to 1 mL |

| | | | |
|---|---|---|---|
| Nitrogen NF | Inert atmosphere | N/A | Inert atmosphere |

### Example 7. Long-Term Stability Assessment of Ready-to-Use Fludarabine Phosphate Composition

Long-term stability of a composition prepared by the method consistent with the present disclosure, such as a composition of Example 1, Example 2, Example 3, Example 4, Example 5, or Example 6, is determined.

Type I glass vials including 2 mL each of the test composition are stored at 5° C.+/−3° C. in both upright and inverted positions. Samples are tested according to the schedule in Table 9, below.

TABLE 9
Long Term Stability Test Points

| Long Term (5° C. ± 3° C.) | Time Points | | | | | | |
|---|---|---|---|---|---|---|---|
| Storage Conditions | T0 | 3 mos. | 6 mos. | 9 mos. | 12 mos. | 18 mos. | 24 mos. |
| Appearance | X | X | X | X | X | X | X |
| Clarity | X | X | X | X | X | X | X |
| pH | X | X | X | X | X | X | X |
| Assay | X | X | X | X | X | X | X |
| Organic Impurities: Early- | X | X | X | X | X | X | X |
| Eluted Impurities | | | | | | | |
| Organic Impurities: Late- | X | X | X | X | X | X | X |
| Eluted Impurities | | | | | | | |
| Sub-Visible Particulate | X | X | | | X | | X |
| Matter | | | | | | | |
| Bacterial Endotoxins Test | X | | | | X | | X |
| Sterility | X | | | | X | | X |
| Container Closure | X | | | | X | | X |
| Integrity Test | | | | | | | |

Appearance, clarity, pH, sub-visible particulate matter, bacterial endotoxins, and container closure integrity are determined by standard methods known in the art. Sterility is determined by USP <71> method. Bioburden (total yeasts and molds and total viable aerobic count) are determined by USP <61> method.

Assay (fludarabine phosphate concentration) is determined by HPLC equipped with a C18 column (e.g., Discovery® C18, 5 μm, 250×4.6 mm), and a UV detector set to 260 nm wavelength. Mobile phase may be, for example, a mixture of 3 parts methanol to 47 parts of a solution including 6.9 g/L monobasic sodium phosphate monohydrate in water (e.g., 50 mM), adjusted to pH 4.5+/−0.2 with 1.0 N sodium hydroxide. The column temperature may be about room temperature and the flow rate may be about 1.0 mL/min. Fludarabine phosphate has a retention time of about 8 minutes in such example conditions.

Sample solutions at 0.1 mg/mL may be prepared, for example, by diluting, with sample solvent to a total volume of 50.0 mL, a 2.0 mL aliquot of a combination of the contents of five vials of fludarabine phosphate for injection 50 mg/vial, and then diluting a 2.0 mL aliquot of that solution to a total volume of 20.0 mL using sample solvent.

Reference standard solutions at 0.1 mg/mL may be prepared, for example, by diluting 10.0 mg of fludarabine phosphate reference standard with 20.0 mL of sample solvent, then diluting a 2.0 mL aliquot of that stock solution to a total volume of 10.0 mL with sample solvent

The symmetry factor (S_{f}) of the fludarabine phosphate peak may be calculated by the equation below:

$${S}_{f} = \frac{{W}_{0.05}}{2 A}$$

where:
   W_{0.05}=width of the peak at one-twentieth of the peak height;
   A=distance between the perpendicular line dropped from the peak at one-twentieth of the peak height

The symmetry factor in some embodiments is not more than 1.8.

The percentage of the labeled amount of fludarabine phosphate in the tested sample may be calculated using the equation below:

$$Percentage of label amt = \frac{ru}{rs} \times \frac{Cs}{Cu} \times 100$$

where:
   ru=peak response from the sample solution;
   rs=peak response from the standard solution;
   Cs=concentration of USP fludarabine phosphate RS in the standard solution (mg/mL); and
   Cu=nominal concentration of fludarabine phosphate in the sample solution (mg/mL).

### Method A: Early-Eluted Impurities

Early-eluted impurities are determined by HPLC equipped with a C18 column (e.g., Phenomenex Luna® C18 (2), 5 μm, 150×4.6 mm), and a UV detector set to 260 nm wavelength. Mobile phase may be, for example, 6% v/v methanol in 0.01 M potassium dihydrogen phosphate. The column temperature may be about room temperature and the flow rate may be about 1.0 mL/min. Fludarabine has a retention time of about 8-9 minutes in such example conditions.

Impurity concentrations are calculated according to the equation below:

$$impurity \% = \frac{Ru}{Rs} \times ( 1 / F 1 ) \times 100$$

where:
   F1=Factor given for tabulated impurities in Table 10 below, and is equal to 1 for any other impurity;
   Ru=Individual impurity peak area in the HPLC chromatogram obtained from a 0.02 mg/mL test solution prepared by, for example, combining 48.0 mL mobile phase and a 2.0 mL aliquot of a stock solution of 10 mg Fludarabine Phosphate Reference Standard dissolved in 20 mL of mobile phase; and
   Rs=Fludarabine phosphate peak area in the HPLC chromatogram obtained from the fludarabine phosphate reference standard test solution described above.

TABLE 10
Early-eluting Impurities

| Impurity | F1* | RRT** |
|---|---|---|
| Iso-ara-guanine-monophosphate (Impurity A) | 0.25 | 0.26 |
| Isoguanine (Impurity B) | 0.40 | 0.34 |
| 3′,5′-Diphosphate analog (Impurity C) | 1.0 | 0.42 |

*Factor value obtained from USP monograph.
**Relative retention time, approximated from USP monograph.

### Method B: Late-Eluted Impurities

Late-eluted impurities are determined by HPLC equipped with a C18 column (e.g., Phenomenex Luna® C18 (2), 5 μm, 150-4.6 mm), and a UV detector set to 260 nm wavelength. Mobile phase may be, for example, 20% v/v methanol in 0.01 M potassium dihydrogen phosphate. The column temperature may be about room temperature and the flow rate may be about 1.0 mL/min. Fludarabine has a retention time of about 3 minutes in such example conditions.

Impurity concentrations are calculated according to the equation below:

$$impurity \% = \frac{Ru}{Rs} \times ( 1 / F 2 ) \times 100$$

where:
   F2=Factor given for tabulated impurities in Table 11 below, and is equal to 1 for any other impurity;
   Ru=Individual impurity peak area in the HPLC chromatogram obtained from a 0.02 mg/mL test solution prepared by, for example, combining 48.0 mL mobile phase and a 2.0 mL aliquot of a stock solution of 10 mg Fludarabine Phosphate Reference Standard dissolved in 20 mL of mobile phase; and
   Rs=Fludarabine phosphate peak area in the HPLC chromatogram obtained from the test solution.

TABLE 11
Late-eluting Impurities

| Impurity | F2 | RRT** |
|---|---|---|
| Fludarabine phosphate | — | 1.0 |
| 2-Fluoroadenine (Impurity D) | 2.0 | 1.5 |
| 2-Fluoro-ara-adenine (Impurity E) | 1.7 | 1.9 |
| 2-Ethoxyphosphate analog (Impurity F) | 0.56* | 2.5 |
| Any individual unspecified impurity | 1.0 | >1.0 |

*Factor value for 2-ethoxyphosphate analog (Impurity F) obtained from Fludarabine phosphate USP monograph (Late Eluting impurities): Fludarabine Phosphate Injection Official Monograph, USP 41-NF 36, pages 1770-1771.
**Relative retention time, approximated from USP monograph.

### Example 8. Accelerated Stability Assessment of Ready-to-Use Fludarabine Phosphate Composition

Stability of a composition prepared by the method consistent with the present disclosure, such as a composition of Example 1, Example 2, Example 3, Example 4, Example 5, or Example 6, is determined under accelerated conditions.

Type I glass vials including 2 mL each of the test composition are stored at 25° C.+/−2° C. and 60%+/−5% relative humidity (RH) in both upright and inverted positions. Samples are tested according to the schedule in Table 12, below.

TABLE 12
Stability Test Points

| Accelerated Storage Conditions | Time Points | | | | |
|---|---|---|---|---|---|
| (25° C. ± 2° C. and 60% +/− 5% RH) | T0 | 1 mo. | 2 mos. | 3 mos. | 6 mos. |
| Appearance | X | X | X | X | X |
| Clarity | X | X | X | X | X |
| pH | X | X | X | X | X |
| Assay | X | X | X | X | X |
| Organic Impurities: Early-Eluted Impurities | X | X | X | X | X |
| Organic Impurities: Late-Eluted Impurities | X | X | X | X | X |
| Sub-Visible Particulate Matter | X | X | | X | X |
| Bacterial Endotoxins Test | X | | | X | X |
| Sterility | X | | | X | X |
| Container Closure Integrity Test | X | | | X | X |

Appearance, clarity, pH, sub-visible particulate matter, bacterial endotoxins, and container closure integrity are determined by standard methods known in the art. Sterility is determined using USP <71> protocol. Bioburden (total yeasts and molds and total viable aerobic count) are determined by USP <61> method. Assay, early-eluted impurities and late-eluted impurities are determined consistent with the methods described in Example 7.

### Example 9. Ready-to-Use Fludarabine Phosphate Compositions in Multi-Dose Vials

Eight ready-to-use compositions of fludarabine phosphate were prepared in glass vials consistent with the method of Example 3. Briefly, 50-mL batches of each test composition were prepared by purging about 40 mL water for injection with nitrogen gas for about 10 minutes. The purged water was then heated to about 70° C. and the methyl paraben and/or propyl paraben was added under continuous stirring. Mannitol or sorbitol was then added at room temperature under continuous stirring. After the preservative(s) and tonicity agent fully dissolved, fludarabine phosphate was added at room temperature under continuous stirring. If needed, sodium hydroxide was then added to bring the pH to 6.8. Additional water for injection was then added to bring the batch to the required total volume. After stirring the batch for about 5 minutes, the ready-to-use fludarabine phosphate solution was aliquoted into 2-mL clear glass vials (Schott Poonawalla) and capped with 13-mm bromobutyl stoppers including ETFE film liners (C1886 6720GC 6 TP3; Aptar Pharma) with 13-mm flip-off seals under nitrogen blanketing. The eight formulations, Formulations XIIIa to XIIIh, had the parameters listed in Table 13:

TABLE 13
Formula Card.

| | | Amount (mg/mL unless otherwise noted) | | | | | | | |
|---|---|---|---|---|---|---|---|---|---|
| | | Formulation: | | | | | | | |
| Component | Function | XIIIa | XIIIb | XIIIc | XIIId | XIIIe | XIIIf | XIIIg | XIIIh |
| Fludarabine | Active agent | 25 | 25 | 25 | 30 | 30 | 30 | 30 | 30 |
| phosphate, USP | | | | | | | | | |
| Mannitol, USP | Tonicity agent | 25 | 0 | 25 | 40 | 0 | 25 | 25 | 0 |
| Sorbitol, USP | Tonicity agent | 0 | 40 | 0 | 0 | 40 | 0 | 0 | 40 |
| Methyl paraben | Preservative | 1.8 | 1.8 | 0 | 0 | 0 | 0 | 1.8 | 1.8 |
| Propyl paraben | Preservative | 0.2 | 0.2 | 0 | 0 | 0 | 0 | 0.2 | 0.2 |
| Benzyl alcohol | Preservative | 0 | 0 | 25 | 0 | 0 | 25 | 0 | 0 |

| Sodium | pH adjuster | q.s. to pH 6.8 |
|---|---|---|
| hydroxide, USP | | |
| Water for | Solvent | q.s. to 1 mL |
| injection | | |
| Nitrogen NF | Inert | Inert atmosphere (headspace) |
| | atmosphere | |

### Example 10. Stability Assessment of Ready-to-Use Fludarabine Phosphate Compositions in Multi-Dose Vials

Stability of a variety of ready-to-use compositions of the ready-to-use fludarabine phosphate compositions in multi-dose vials of Example 9 (i.e., Formulations XIIIa to XIIIh) was assessed under refrigerated, real-time, and accelerated storage conditions.

Briefly, the vials prepared in Example 9 were stored at 2-8° C., at 25° C. and 60% relative humidity, or at 40° C. and 75% relative humidity. Characteristics of each stored formulation before storage (T0), after one week (T=1W), after 2 weeks (T=2W), and after 4 weeks (T=4W) are shown in Tables 14-21, below; characteristics of Formulations XIIIb, XIIIf, XIIIg, and XIIIh after three months (3M) are also shown in Tables 15 and 19-21, respectively:

TABLE 14
Characteristics of Formula XIIIa After Storage.

| | T0 | T = 1 W | T = 2 W | T = 4 W |
|---|---|---|---|---|
| 2-8° C. | | | | |
| Description | Clear | Not tested | Not tested | Clear |
| | colorless | | | colorless |
| | solution | | | solution |
| Assay | 101.3% | Not tested | Not tested | 102.8% |
| pH | 6.9 | Not tested | Not tested | 7.04 |
| Osmolality | 398 | Not tested | Not tested | Not tested |

Early-Eluting Impurities (Method A)

| | | | | |
|---|---|---|---|---|
| iso araguanine | 0.3% | Not tested | Not tested | 0.28% |
| monophosphate | | | | |
| iso guanine | ND | Not tested | Not tested | <0.05% |
| 3,5 diphosphate analog | 0.01% | Not tested | Not tested | <0.05% |
| Any other early-eluting | 0.01% | Not tested | Not tested | <0.05% |
| impurity | | | | |

Late-Eluting Impurities (Method B)

| | | | | |
|---|---|---|---|---|
| 2-fluoroadenine | 0.02% | Not tested | Not tested | <0.05% |
| 2-fluoroara adenine | ND | Not tested | Not tested | ND |
| 2-Ethoxyphosphate analog | 0.03% | Not tested | Not tested | ND |
| Any other late-eluting | <0.05% | Not tested | Not tested | <0.05% |
| impurity | | | | |
| Total Impurities (A + B) | 0.3% | Not tested | Not tested | 0.3% |
| 25° C./60RH | | | | |
| Description | Clear | Clear | Clear | Clear |
| | colorless | colorless | colorless | colorless |
| | solution | solution | solution | solution |
| Assay | 101.3% | 101.7% | 101.7% | 103% |
| pH | 6.9 | Not done | Not done | 6.8 |
| Osmolality | 398 | Not done | Not done | Not tested |

Early-Eluting Impurities (Method A)

| | | | | |
|---|---|---|---|---|
| iso araguanine | 0.3% | 0.2% | 0.28% | 0.3% |
| monophosphate | | | | |
| iso guanine | ND | ND | ND | <0.05% |
| 3,5 diphosphate analog | 0.01% | ND | 0.01% | <0.05% |
| Any other early-eluting | 0.01% | 0.02% | 0.01% | <0.05% |
| impurity | | | | |

Late-Eluting Impurities (Method B)

| | | | | |
|---|---|---|---|---|
| 2-fluoroadenine | 0.02% | ND | 0.03% | 0.02% |
| 2-fluoroara adenine | 0.02% | ND | ND | ND |
| 2-Ethoxyphosphate analog | ND | ND | ND | ND |
| Any other late-eluting | 0.03% | 0.07%(RRT 2.00) | 0.08%(RRT −1.3) | 0.03%(RRT1.03) |
| impurity | | | 0.07%(RRT 2.05) | 0.11%(RRT2.05) |
| | | | | 0.03%(RRT2.29) |
| Total Impurities (A + B) | 0.3% | 0.4% | 0.3% | 0.4% |
| 40° C./75RH | | | | |
| Description | Clear | Clear | Clear | Clear |
| | colorless | colorless | colorless | colorless |
| | solution | solution | solution | solution |
| Assay | 101.3% | 99.4% | 102.9% | 103% |
| pH | 6.9 | Not done | Not done | 7.05 |
| Osmolality | 398 | Not done | Not done | Not tested |

Early-Eluting Impurities (Method A)

| | | | | |
|---|---|---|---|---|
| iso araguanine | 0.3% | 0.36% | 0.48% | 0.71% |
| monophosphate | | | | |
| iso guanine | ND | ND | ND | 0.05% |
| 3,5 diphosphate analog | 0.01% | ND | 0.01% | <0.05% |
| Any other early-eluting | 0.01% | 0.02% | 0.04% (RRT 0.2) | 0.07%(RRT−0.2) |
| impurity | | | | 0.05%(RRT −0.8) |

Late-Eluting Impurities (Method B)

| | | | | |
|---|---|---|---|---|
| 2-fluoroadenine | 0.02% | ND | 0.025% | 0.03% |
| 2-fluoroara adenine | 0.02% | ND | ND | 0.16% |
| 2-Ethoxyphosphate analog | ND | ND | ND | ND |
| Any other late-eluting | 0.03% | 0.05% (RRT1.55) | 0.06% (RRT1.3) | 0.03%(RRT1.3) |
| impurity | | 0.19% (RRT1.98) | 0.35% (RRT2.05) | 0.31%(RRT2.05) |
| | | 0.05% (RRT2.22) | 0.09% (RRT2.28) | 0.17%(RRT2.29) |
| Total Impurities (A + B) | 0.3% | 0.6% | 1.00% | 1.52% |

ND = Not Detected
RRT = Relative Retention Time

TABLE 15
Characteristics of Formula XIIIb After Storage.

| | T0 | T = 1 W | T = 2 W | T = 4 W | T = 3 M |
|---|---|---|---|---|---|
| 2-8° C. | | | | | |
| Description | Clear | Not tested | Not tested | Clear | Clear |
| | colorless | | | colorless | colorless |
| | solution | | | solution | solution |
| Assay | 98.2% | Not tested | Not tested | 104.8% | 101% |
| pH | 6.93 | Not tested | Not tested | 6.99 | 6.8 |
| Osmolality | 302 | Not tested | Not tested | Not tested | Not tested |
| Early-Eluting Impurities (Method A) | | | | | |
| iso araguanine | 0.3% | Not tested | Not tested | 0.31% | 0.3% |
| monophosphate | | | | | |
| iso guanine | ND | Not tested | Not tested | <0.05% | 0.03% |
| 3,5 diphosphate | <0.05% | Not tested | Not tested | <0.05% | ND |
| analog | | | | | |
| Any other early- | <0.05% | Not tested | Not tested | <0.05% | ND |
| eluting impurity | | | | | |
| Late-Eluting Impurities (Method B) | | | | | |
| 2-fluoroadenine | 0.02% | Not tested | Not tested | <0.05% | 0.03% |
| 2-fluoroara | 0.02% | Not tested | Not tested | ND | 0.01% |
| adenine | | | | | |
| 2- | ND | Not tested | Not tested | ND | ND |
| Ethoxyphosphate | | | | | |
| analog | | | | | |
| Any other late- | 0.04% | Not tested | Not tested | 0.12%(RRT−2.05) | 0.1% |
| eluting impurity | | | | | |
| Total Impurities | 0.3% | Not tested | Not tested | 0.43% | 0.4% |
| (A + B) | | | | | |
| 25° C./60RH | | | | | |
| Description | Clear | Clear | Clear | Clear | Clear |
| | colorless | colorless | colorless | colorless | colorless |
| | solution | solution | solution | solution | solution |
| Assay | 98.2% | 98.9% | 101.2% | 101.2% | 99.7% |
| pH | 6.93 | Not done | Not done | 6.93 | 6.9 |
| Osmolality | 302 | Not done | Not done | Not tested | Not tested |
| Early-Eluting Impurities (Method A) | | | | | |
| iso araguanine | 0.3% | 0.2% | 0.32% | 0.3% | 0.4% |
| monophosphate | | | | | |
| iso guanine | ND | ND | ND | <0.05% | 0.05% |
| 3,5 diphosphate | 0.01% | ND | 0.01% | <0.05% | ND |
| analog | | | | | |
| Any other early- | 0.01% | 0.01% | 0.01% | <0.05% | ND |
| eluting impurity | | | | | |
| Late-Eluting Impurities (Method B) | | | | | |
| 2-fluoroadenine | 0.02% | ND | 0.025% | 0.02% | 0.03% |
| 2-fluoroara | 0.02% | ND | ND | ND | 0.04% |
| adenine | | | | | |
| 2- | ND | ND | ND | ND | ND |
| Ethoxyphosphate | | | | | |
| analog | | | | | |
| Any other late- | 0.04% | 0.06% | 0.08%(RRT1.3) | 0.03%(RRT1.3) | .03% |
| eluting impurity | | | 0.07%(RRT2.05) | 0.07%(RRT2.05) | |
| Total Impurities | 0.3% | 0.3% | 0.51% | 0.4% | 0.8% |
| (A + B) | | | | | |
| 40° C./75RH | | | | | |
| Description | Clear | Clear | Clear | Clear | Not tested |
| | colorless | colorless | colorless | colorless | |
| | solution | solution | solution | solution | |
| Assay | 98.2% | 98.0% | 100.0% | 101.2% | Not tested |
| pH | 6.93 | Not done | Not done | 7.04 | Not tested |
| Osmolality | 302 | Not done | Not done | Not tested | Not tested |
| Early-Eluting Impurities (Method A) | | | | | |
| iso araguanine | 0.3% | 0.3% | 0.52% | 0.82% | Not tested |
| monophosphate | | | | | |
| iso guanine | ND | ND | 0.01% | 0.05% | Not tested |
| 3,5 diphosphate | 0.01% | ND | 0.01% | <0.05% | Not tested |
| analog | | | | | |
| Any other early- | 0.01% | 0.03% | 0.04%(RRT−0.2) | 0.07%(RRT−0.2) | Not tested |
| eluting impurity | | | | 0.05%(RRT−0.8) | |
| Late-Eluting Impurities (Method B) | | | | | |
| 2-fluoroadenine | 0.02% | ND | 0.03% | 0.03% | |
| 2-fluoroara | 0.02% | ND | ND | 0.165% | |
| adenine | | | | | |
| 2- | ND | ND | ND | ND | |
| Ethoxyphosphate | | | | | |
| analog | | | | | |
| Any other late- | 0.04% | 0.19% | 0.04%(RRT1.3) | 0.03%(RRT1.3) | |
| eluting impurity | | | 0.35%(RRT2.05) | 0.31%(RRT2.05) | |
| | | | 0.09%(RRT2.28) | 0.14%(RRT2.35) | |
| Total Impurities (A + B) | 0.3% | 0.6% | 0.8% | 1.6% | |

ND = Not Detected
RRT = Relative Retention Time

TABLE 16
Characteristics of Formula XIIIc After Storage.

| | T0 | T = 1 W | T = 2 W | T = 4 W |
|---|---|---|---|---|
| 2-8° C. | | | | |
| Description | Clear | Not tested | Not tested | Clear |
| | colorless | | | colorless |
| | solution | | | solution |
| Assay | 98.7% | Not tested | Not tested | 102.6% |
| pH | 6.95 | Not tested | Not tested | 6.94 |
| Osmolality | 393 | Not tested | Not tested | Not tested |

Early-Eluting Impurities (Method A)

| | | | | |
|---|---|---|---|---|
| iso araguanine | 0.3% | Not tested | Not tested | 0.29% |
| monophosphate | | | | |
| iso guanine | ND | Not tested | Not tested | <0.05% |
| 3,5 diphosphate analog | 0.01% | Not tested | Not tested | <0.05% |
| Any other early-eluting | 0.01% | Not tested | Not tested | <0.05% |
| impurity | | | | |

Late-Eluting Impurities (Method B)

| | | | | |
|---|---|---|---|---|
| 2-fluoroadenine | 0.02% | Not tested | Not tested | <0.05% |
| 2-fluoroara adenine | 0.02% | Not tested | Not tested | ND |
| 2-Ethoxyphosphate analog | ND | Not tested | Not tested | ND |
| Any other late-eluting | 0.03% | Not tested | Not tested | 0.12% |
| impurity | | | | |
| Total Impurities (A + B) | 0.3% | Not tested | Not tested | 0.41% |
| 25° C./60RH | | | | |
| Description | Clear | Clear | Clear | Clear |
| | colorless | colorless | colorless | colorless |
| | solution | solution | solution | solution |
| Assay | 98.7% | 100.0% | 100.7% | 102.5% |
| pH | 6.95 | Not done | Not done | 6.92 |
| Osmolality | 393 | Not done | Not done | Not tested |

Early-Eluting Impurities (Method A)

| | | | | |
|---|---|---|---|---|
| iso araguanine | 0.3% | 0.2% | 0.28% | 0.3% |
| monophosphate | | | | |
| iso guanine | ND | ND | ND | <0.05% |
| 3,5 diphosphate analog | 0.01% | ND | 0.01% | <0.05% |
| Any other early-eluting | 0.01% | 0.01% | 0.01% | <0.05% |
| impurity | | | | |

Late-Eluting Impurities (Method B)

| | | | | |
|---|---|---|---|---|
| 2-fluoroadenine | 0.02% | ND | 0.025% | 0.02% |
| 2-fluoroara adenine | 0.02% | ND | ND | ND |
| 2-Ethoxyphosphate analog | ND | ND | ND | ND |
| Any other late-eluting | 0.03% | 0.07% | 0.09%(RRT2.05) | 0.03%(RRT1.3) |
| impurity | | | | 0.1%(RRT2.05) |
| Total Impurities (A + B) | 0.3% | 0.4% | 0.42% | 0.4% |
| 40° C./75RH | | | | |
| Description | Clear | Clear | Clear | Clear |
| | colorless | colorless | colorless | colorless |
| | solution | solution | solution | solution |
| Assay | 98.7% | 98.2% | 100.8% | 102.5% |
| pH | 6.95 | Not done | Not done | 6.91 |
| Osmolality | 393 | Not done | Not done | Not tested |

Early-Eluting Impurities (Method A)

| | | | | |
|---|---|---|---|---|
| iso araguanine | 0.3% | 0.32% | 0.48% | 0.61% |
| monophosphate | | | | |
| iso guanine | ND | ND | 0.01% | 0.08% |
| 3,5 diphosphate analog | 0.01% | ND | 0.01% | <0.05% |
| Any other early-eluting | 0.01% | 0.03% | 0.04%(RRT 0.2) | 0.07%(RRT0.2) |
| impurity | | | | |

Late-Eluting Impurities (Method B)

| | | | | |
|---|---|---|---|---|
| 2-fluoroadenine | 0.02% | ND | 0.03% | 0.03% |
| 2-fluoroara adenine | 0.02% | ND | ND | ND |
| 2-Ethoxyphosphate analog | ND | ND | ND | ND |
| Any other late-eluting | 0.03% | 0.13% | 0.06%(RRT1.3) | 0.03%(RRT1.3) |
| impurity | | | 0.22%(RRT2.05) | 0.35%(RRT2.05) |
| | | | 0.01%(RRT2.28) | |
| MTotal Impurities (A + B) | 0.3% | 0.5% | 0.85% | 1.11% |

ND = Not Detected
RRT = Relative Retention Time

TABLE 17
Characteristics of Formula XIIId After Storage.

| | T0 | T = 1 W | T = 2 W | T = 4 W |
|---|---|---|---|---|
| 2-8° C. | | | | |
| Description | Clear | Not tested | Not tested | Clear |
| | colorless | | | colorless |
| | solution | | | solution |
| Assay | 98.9% | Not tested | Not tested | 99.1% |
| pH | 6.9 | Not tested | Not tested | Not tested |
| Osmolality | 300 | Not tested | Not tested | Not tested |

Early-Eluting Impurities (Method A)

| | | | | |
|---|---|---|---|---|
| iso araguanine | 0.3% | Not tested | Not tested | 0.2% |
| monophosphate | | | | |
| iso guanine | ND | Not tested | Not tested | ND |
| 3,5 diphosphate analog | 0.01% | Not tested | Not tested | 0.01% |
| Any other early-eluting | 0.01% | Not tested | Not tested | 0.02% |
| impurity | | | | |

Late-Eluting Impurities (Method B)

| | | | | |
|---|---|---|---|---|
| 2-fluoroadenine | 0.02% | Not tested | Not tested | ND |
| 2-fluoroara adenine | 0.02% | Not tested | Not tested | ND |
| 2-Ethoxyphosphate analog | ND | Not tested | Not tested | ND |
| Any other late-eluting | 0.03% | Not tested | Not tested | 0.16% |
| impurity | | | | |
| Total Impurities (A + B) | 0.03% | Not tested | Not tested | 0.4% |
| 25° C./60RH | | | | |
| Description | Clear | Clear | Clear | Clear |
| | colorless | colorless | colorless | colorless |
| | solution | solution | solution | solution |
| Assay | 98.9% | 99.1% | 100.2% | 102.7% |
| pH | 6.9 | Not done | Not done | 6.92 |
| Osmolality | 300 | Not done | Not done | Not tested |

Early-Eluting Impurities (Method A)

| | | | | |
|---|---|---|---|---|
| iso araguanine | 0.3% | 0.2% | 0.28% | 0.28% |
| monophosphate | | | | |
| iso guanine | ND | ND | ND | <0.05% |
| 3,5 diphosphate analog | 0.01% | 0.01% | 0.01% | <0.05% |
| Any other early-eluting | 0.01% | 0.02% | 0.02% | <0.05% |
| impurity | | | | |

Late-Eluting Impurities (Method B)

| 2-fluoroadenine | 0.02% | ND | 0.025% | <0.05% |
|---|---|---|---|---|
| 2-fluoroara adenine | 0.02% | ND | ND | ND |
| 2-Ethoxyphosphate analog | ND | ND | ND | ND |
| Any other late-eluting | 0.03% | 0.16% | 0.07%(RRT1.3) | 0.05% |
| impurity | | | 1.44%(RRT2.05) | |
| Total Impurities (A + B) | 0.3% | 0.4% | 1.845% | 0.33% |
| 40° C./75RH | | | | |
| Description | Clear | Clear | Clear | Clear |
| | colorless | colorless | colorless | colorless |
| | solution | solution | solution | solution |
| Assay | 98.9% | 98.6% | 102.8% | 102.4% |
| pH | 6.9 | Not done | Not done | 6.92 |
| Osmolality | 300 | Not done | Not done | Not tested |
| Early-Eluting Impurities (Method A) | | | | |
| iso araguanine | 0.3% | 0.3% | 0.48% | 0.83% |
| monophosphate | | | | |
| iso guanine | ND | ND | 0.01% | 0.07% |
| 3,5 diphosphate analog | 0.01% | ND | 0.01% | <0.05% |
| Any other early-eluting | 0.01% | 0.03% | 0.04%(RRT 0.2) | 0.07%(RRT0.2) |
| impurity | | | | 0.06%(RRT0.8) |
| Late-Eluting Impurities (Method B) | | | | |
| 2-fluoroadenine | 0.02% | ND | 0.03% | 0.02% |
| 2-fluoroara adenine | 0.02% | ND | ND | ND |
| 2-Ethoxyphosphate analog | ND | ND | ND | ND |
| Any other late-eluting | 0.03% | 0.13% | 0.06%(RRT1.3) | 0.33%(RRT2.05) |
| impurity | | | 0.16%(RRT2.05) | 0.04%(RRT2.3) |
| | | | 0.03%(RRT2.28) | |
| Total Impurities (A + B) | 0.3% | 0.5% | 0.8% | 1.36% |

ND = Not Detected
RRT = Relative Retention Time

TABLE 18
Characteristics of Formula XIIIe After Storage.

| | T0 | T = 1 W | T = 2 W | T = 4 W |
|---|---|---|---|---|
| 2-8° C. | | | | |
| Description | Clear | Not tested | Not tested | Clear |
| | colorless | | | colorless |
| | solution | | | solution |
| Assay | 99.4% | Not tested | Not tested | 101.3% |
| pH | 6.82 | Not tested | Not tested | 6.96 |
| Osmolality | 393 | Not tested | Not tested | Not tested |

Early-Eluting Impurities (Method A)

| | | | | |
|---|---|---|---|---|
| iso araguanine | 0.3% | Not tested | Not tested | 0.27% |
| monophosphate | | | | |
| iso guanine | ND | Not tested | Not tested | <0.05% |
| 3,5 diphosphate analog | <0.05% | Not tested | Not tested | <0.05% |
| Any other early-eluting | <0.05% | Not tested | Not tested | <0.05% |
| impurity | | | | |

Late-Eluting Impurities (Method B)

| | | | | |
|---|---|---|---|---|
| 2-fluoroadenine | <0.05% | Not tested | Not tested | <0.05% |
| 2-fluoroara adenine | <0.05% | Not tested | Not tested | ND |
| 2-Ethoxyphosphate analog | ND | Not tested | Not tested | ND |
| Any other late-eluting | <0.05% | Not tested | Not tested | <0.05% |
| impurity | | | | |
| Total Impurities (A + B) | 0.3% | Not tested | Not tested | 0.27% |
| 25° C./60RH | | | | |
| Description | Clear | Clear | Clear | Clear |
| | colorless | colorless | colorless | colorless |
| | solution | solution | solution | solution |
| Assay | 99.4% | 98.8% | 97.9% | 101.4% |
| pH | 6.82 | Not done | Not done | 6.92 |
| Osmolality | 392 | Not done | Not done | Not tested |

Early-Eluting Impurities (Method A)

| | | | | |
|---|---|---|---|---|
| iso araguanine | 0.3% | 0.3% | 0.32% | 0.3% |
| monophosphate | | | | |
| iso guanine | ND | ND | ND | <0.05% |
| 3,5 diphosphate analog | 0.01% | 0.01% | 0.02% | <0.05% |
| Any other early-eluting | 0.01% | 0.01% | 0.01% | <0.05% |
| impurity | | | | |

Late-Eluting Impurities (Method B)

| | | | | |
|---|---|---|---|---|
| 2-fluoroadenine | 0.02% | ND | 0.025% | 0.02% |
| 2-fluoroara adenine | 0.02% | ND | ND | ND |
| 2-Ethoxyphosphate analog | ND | ND | ND | ND |
| Any other late-eluting | 0.04% | 1.42%(RRT2.05) | 0.06%(RRT1.3) | 0.04%(RRT1.3) |
| impurity | | | 3.1%(RRT2.05 ) | 3.1%(RRT2.05 ) |
| Total Impurities (A + B) | 0.3% | 1.7% | 3.5% | 3.4% |
| 40° C./75RH | | | | |
| Description | Clear | Clear | Clear | Clear |
| | colorless | colorless | colorless | colorless |
| | solution | solution | solution | solution |
| Assay | 99.4% | 98.4% | 101.2% | 101.4% |
| pH | 6.82 | Not done | Not done | 6.92 |
| Osmolality | 392 | Not done | Not done | Not tested |

Early-Eluting Impurities (Method A)

| | | | | |
|---|---|---|---|---|
| iso araguanine | 0.3% | 0.3% | 0.52% | 0.45% |
| monophosphate | | | | |
| iso guanine | ND | ND | 0.01% | <0.05% |
| 3,5 diphosphate analog | 0.01% | ND | 0.01% | <0.05% |
| Any other early-eluting | 0.01% | 0.03% | <0.05% | 0.05% |
| impurity | | | | |

Late-Eluting Impurities (Method B)

| | | | | |
|---|---|---|---|---|
| 2-fluoroadenine | 0.02% | ND | 0.025% | 0.02% |
| 2-fluoroara adenine | 0.02% | ND | ND | ND |
| 2-Ethoxyphosphate analog | ND | ND | ND | ND |
| Any other late-eluting | 0.04% | 0.42% | 0.05%(RRT1.3) | 0.03%(RRT1.3) |
| impurity | | | 0.21%(RRT2.05) | 1.14%(RRT2.05) |
| | | | 0.03%(RRT2.28) | 0.04%(RRT2.29) |
| Total Impurities (A + B) | 0.3% | 0.8% | 0.9% | 1.6% |

ND = Not Detected
RRT = Relative Retention Time

TABLE 19
Characteristics of Formula XIIIf After Storage.

| | T0 | T = 1 W | T = 2 W | T = 4 W | T = 3 M |
|---|---|---|---|---|---|
| 2-8° C. | | | | | |
| Description | Clear | Not tested | Not tested | Clear | Clear |
| | colorless | | | colorless | colorless |
| | solution | | | solution | solution |
| Assay | 97.9% | Not tested | Not tested | 102.2% | 101.3% |
| pH | 6.92 | Not tested | Not tested | 6.92 | 6.8 |
| Osmolality | 512 | Not tested | Not tested | Not tested | Not tested |
| Early-Eluting Impurities (Method A) | | | | | |
| iso araguanine | 0.3% | Not tested | Not tested | 0.28% | 0.2% |
| monophosphate | | | | | |
| iso guanine | ND | Not tested | Not tested | 0.018% | 0.04% |
| 3,5 diphosphate | 0.01% | Not tested | Not tested | 0.012% | ND |
| analog | | | | | |
| Any other early- | <0.05% | Not tested | Not tested | <0.05% | ND |
| eluting impurity | | | | | |
| Late-Eluting Impurities (Method B) | | | | | |
| 2-fluoroadenine | 0.02% | Not tested | Not tested | 0.022% | 0.03% |
| 2-fluoroara adenine | 0.02% | Not tested | Not tested | ND | ND |
| 2-Ethoxyphosphate | ND | Not tested | Not tested | ND | ND |
| analog | | | | | |
| Any other late- | 0.04% | Not tested | Not tested | <0.05% | 0.05% |
| eluting impurity | | | | | |
| Total Impurities | 0.3% | Not tested | Not tested | 0.28% | 0.3% |
| (A + B) | | | | | |
| 25° C./60RH | | | | | |
| Description | Clear | Clear | Clear | Clear | Clear |
| | colorless | colorless | colorless | colorless | colorless |
| | Solution | Solution | Solution | solution | solution |
| Assay | 97.9% | 98.4% | 100.2% | 102% | 99.8% |
| pH | 6.92 | Not done | Not done | 6.94 | 6.9 |
| Osmolality | 512 | Not done | Not done | Not tested | Not tested |
| Early-Eluting Impurities (Method A) | | | | | |
| iso araguanine | 0.3% | 0.3% | 0.28% | 0.33% | 0.3% |
| monophosphate | | | | | |
| iso guanine | ND | ND | ND | 0.017% | 0.05% |
| 3,5 diphosphate | 0.01% | 0.01% | 0.01% | <0.05% | ND |
| analog | | | | | |
| Any other early- | <0.05% | 0.01% | 0.01% | <0.05% | ND |
| eluting impurity | | | | | |
| Late-Eluting Impurities (Method B) | | | | | |
| 2-fluoroadenine | 0.02% | ND | 0.025% | 0.023% | 0.03% |
| 2-fluoroara adenine | 0.02% | ND | ND | ND | ND |
| 2-Ethoxyphosphate | ND | ND | ND | ND | ND |
| analog | | | | | |
| Any other late- | 0.04% | 0.06% | 0.06%(RRT1.3) | 0.05%(RRT2.05) | 0.2% |
| eluting impurity | | | | | |
| Total Impurities | 0.3% | 0.4% | 0.42% | 0.4% | 0.5% |
| (A + B) | | | | | |
| 40° C./75RH | | | | | |
| Description | Clear | Clear | Clear | Clear | Not tested |
| | colorless | colorless | colorless | colorless | |
| | solution | solution | solution | solution | |
| Assay | 97.9% | 98.4% | 99.8% | 101.8% | Not tested |
| pH | 6.92 | Not done | Not done | 6.95 | Not tested |
| Osmolality | 512 | Not done | Not done | Not tested | Not tested |
| Early-Eluting Impurities (Method A) | | | | | |
| iso araguanine | 0.3% | 0.3% | 0.48% | 0.59% | Not tested |
| monophosphate | | | | | |
| iso guanine | ND | ND | 0.01% | 0.03% | Not tested |
| 3,5 diphosphate | 0.01% | ND | 0.01% | 0.018% | Not tested |
| analog | | | | | |
| Any other early- | <0.05% | 0.03% | 0.04%(RRT−0.2) | <0.05% | Not tested |
| eluting impurity | | | | | |
| Late-Eluting Impurities (Method B) | | | | | |
| 2-fluoroadenine | 0.02% | ND | 0.03% | 0.03% | Not tested |
| 2-fluoroara adenine | 0.02% | ND | ND | ND | Not tested |
| 2-Ethoxyphosphate | ND | ND | ND | ND | Not tested |
| analog | | | | | |
| Any other late- | 0.04% | 0.09% | 0.06%(RRT1.3) | 025%(RRT2.05) | Not tested |
| eluting impurity | | | 0.14%(RRT2.05) | | |
| | | | 0.03(RRT2.28) | | |
| Total Impurities | 0.3% | 0.4% | 0.8% | 1.0% | Not tested |
| (A + B) | | | | | |

ND = Not Detected
RRT = Relative Retention Time

TABLE 20
Characteristics of Formula XIIIg After Storage.

| | T0 | T = 1 W | T = 2 W | T = 4 W | T = 3 M |
|---|---|---|---|---|---|
| 2-8° C. | | | | | |
| Description | Clear | Not tested | Not tested | Clear | Clear |
| | colorless | | | colorless | colorless |
| | solution | | | solution | solution |
| Assay | 98.7% | Not tested | Not tested | 102.6% | 101.5% |
| pH | 6.95 | Not tested | Not tested | 6.9 | 6.8 |
| Osmolality | 393 | Not tested | Not tested | Not tested | Not tested |

Early-Eluting Impurities (Method A)

| | | | | | |
|---|---|---|---|---|---|
| iso araguanine | 0.3% | Not tested | Not tested | 0.28% | 0.2% |
| monophosphate | | | | | |
| iso guanine | ND | Not tested | Not tested | 0.029% | ND |
| 3,5 diphosphate | <0.05% | Not tested | Not tested | 0.016% | ND |
| analog | | | | | |
| Any other early- | <0.05% | Not tested | Not tested | <0.05% | ND |
| eluting impurity | | | | | |

Late-Eluting Impurities (Method B)

| 2-fluoroadenine | 0.02% | Not tested | Not tested | 0.023% | 0.03% |
|---|---|---|---|---|---|
| 2-fluoroara adenine | 0.02% | Not tested | Not tested | ND | ND |
| 2-Ethoxyphosphate | ND | Not tested | Not tested | ND | ND |
| analog | | | | | |
| Any other late- | 0.03% | Not tested | Not tested | 0.06%(RRT2.08) | 0.2% |
| eluting impurity | | | | | |
| Total Impurities | 0.3% | Not tested | Not tested | 0.34% | 0.4% |
| (A + B) | | | | | |
| 25° C./60RH | | | | | |
| Description | Clear | Clear | Clear | Clear | Clear |
| | colorless | colorless | colorless | colorless | colorless |
| | solution | solution | solution | solution | solution |
| Assay | 98.6% | 99.3% | 101.1% | 101% | 100.9% |
| pH | 6.89 | Not done | Not done | 6.91 | 6.9 |
| Osmolality | 296 | Not done | Not done | Not tested | Not tested |
| Early-Eluting Impurities (Method A) | | | | | |
| iso araguanine | 0.3% | 0.3% | 0.28% | 0.35% | 0.4% |
| monophosphate | | | | | |
| iso guanine | ND | ND | ND | 0.025% | 0.05% |
| 3,5 diphosphate | 0.01% | 0.01% | 0.01% | <0.05% | ND |
| analog | | | | | |
| Any other early- | 0.01% | 0.01% | 0.01% | <0.05% | ND |
| eluting impurity | | | | | |
| Late-Eluting Impurities (Method B) | | | | | |
| 2-fluoroadenine | 0.02% | ND | 0.025% | 0.023% | 0.03% |
| 2-fluoroara adenine | 0.02% | ND | ND | ND | ND |
| 2-Ethoxyphosphate | ND | ND | ND | ND | ND |
| analog | | | | | |
| Any other late- | 0.04% | 0.11% | 0.15%(RRT2.05) | 0.22%(RRT2.06) | 0.7% |
| eluting impurity | | | | 0.05%(RRT2.29) | |
| Total Impurities | 0.3% | 0.4% | 0.5% | 0.6% | 1.2% |
| (A + B) | | | | | |
| 40° C./75RH | | | | | |
| Description | Clear | Clear | Clear | Clear | Not tested |
| | colorless | colorless | colorless | colorless | |
| | solution | solution | solution | solution | |
| Assay | 98.6% | 99% | 99.9% | 101.8% | Not tested |
| pH | 6.89 | Not done | Not done | 6.93 | Not tested |
| Osmolality | 296 | Not done | Not done | Not tested | Not tested |
| Early-Eluting Impurities (Method A) | | | | | |
| iso araguanine | 0.3% | 0.4% | 0.52% | 0.65% | Not tested |
| monophosphate | | | | | |
| iso guanine | ND | ND | 0.01% | 0.04% | Not tested |
| 3,5 diphosphate | 0.01% | ND | 0.01% | 0.019% | Not tested |
| analog | | | | | |
| Any other early- | 0.01% | 0.03% | 0.04%(RRT 0.2) | <0.05% | Not tested |
| eluting impurity | | | | | |
| Late-Eluting Impurities (Method B) | | | | | |
| 2-fluoroadenine | 0.02% | ND | 0.03% | 0.03% | Not tested |
| 2-fluoroara adenine | 0.02% | ND | ND | <0.05% | Not tested |
| 2-Ethoxyphosphate | ND | ND | ND | ND | Not tested |
| analog | | | | | |
| Any other late- | 0.04% | 0.26% | 0.05%(RRT1.3) | 0.56%(RRT2.05) | Not tested |
| eluting impurity | | | 0.46%(RRT2.05) | 0.34%(RRT2.29) | |
| | | | 0.08%(RRT2.28) | 0.05%(RRT2.56) | |
| Total Impurities | 0.3% | 0.7% | 1.12% | 1.8% | Not tested |
| (A + B) | | | | | |

ND = Not Detected
RRT = Relative Retention Time

TABLE 21
Characteristics of Formula XIIIh After Storage.

| 2-8° C. | T0 | T = 1 W | T = 2 W | T = 4 W | T = 3 M |
|---|---|---|---|---|---|
| Description | Clear | Not tested | Not tested | Clear | Clear |
| | colorless | | | colorless | colorless |
| | solution | | | solution | solution |
| Assay | 98.9% | Not tested | Not tested | 102.3% | 101.7% |
| pH | 6.91 | Not tested | Not tested | 6.91 | 6.9 |
| Osmolality | 524 | Not tested | Not tested | Not tested | Not tested |

Early-Eluting Impurities (Method A)

| | | | | | |
|---|---|---|---|---|---|
| iso araguanine | 0.3% | Not tested | Not tested | 0.28% | 0.3% |
| monophosphate | | | | | |
| iso guanine | ND | Not tested | Not tested | 0.01% | 0.03% |
| 3,5 diphosphate | ND | Not tested | Not tested | 0.01% | ND |
| analog | | | | | |
| Any other early- | <0.05% | Not tested | Not tested | <0.05% | ND |
| eluting impurity | | | | | |

Late-Eluting Impurities (Method B)

| 2-fluoroadenine | 0.02% | Not tested | Not tested | 0.022% | 0.03% |
|---|---|---|---|---|---|
| 2-fluoroara adenine | 0.02% | Not tested | Not tested | ND | ND |
| 2-Ethoxyphosphate | ND | Not tested | Not tested | ND | ND |
| analog | | | | | |
| Any other late- | 0.03% | Not tested | Not tested | <0.05% | 0.1% |
| eluting impurity | | | | | |
| Total Impurities | 0.3% | Not tested | Not tested | 0.28% | 0.4% |
| (A + B) | | | | | |
| 25° C./60RH | T0 | T = 1 W | T = 2 W | T = 4 W | T = 3 M |
| Description | Clear | Clear | Clear | Clear | Clear |
| | colorless | colorless | colorless | colorless | colorless |
| | solution | solution | solution | solution | solution |
| Assay | 98.9% | 99.2% | 100.8% | 102% | 100.7% |
| pH | 6.91 | Not done | Not done | 6.92 | 6.8 |
| Osmolality | 524 | Not done | Not done | Not tested | Not tested |

Early-Eluting Impurities (Method A)

| | | | | | |
|---|---|---|---|---|---|
| iso araguanine | 0.3% | 0.3% | 0.32% | 0.35% | 0.4% |
| monophosphate | | | | | |
| iso guanine | ND | ND | ND | 0.026% | 0.05% |
| 3,5 diphosphate | ND | 0.01% | 0.01% | 0.019% | ND |
| analog | | | | | |
| Any other early- | 0.01% | 0.01% | 0.01% | <0.05% | ND |
| eluting impurity | | | | | |

Late-Eluting Impurities (Method B)

| 2-fluoroadenine | 0.02% | ND | 0.025% | 0.023% | 0.03% |
|---|---|---|---|---|---|
| 2-fluoroara adenine | 0.02% | ND | ND | ND | 0.02% |
| 2-Ethoxyphosphate | ND | ND | ND | ND | ND |
| analog | | | | | |
| Any other late- | 0.03% | 0.08% | 0.06% | 0.09% | 0.2% |
| eluting impurity | | | (RRT2.07) | (RRT2.05) | |
| Total Impurities | 0.3% | 0.4% | 0.4% | 0.4% | 0.70% |
| (A + B) | | | | | |
| 40° C./75RH | T0 | T = 1 W | T = 2 W | T = 4 W | T = 3 M |
| Description | Clear | Clear | Clear | Clear | Not tested |
| | colorless | colorless | colorless | colorless | |
| | solution | solution | solution | solution | |
| Assay | 98.9% | 97.9% | 99.5% | 101.9% | Not tested |
| pH | 6.91 | Not done | Not done | 6.93 | Not tested |
| Osmolality | 524 | Not done | Not done | Not tested | Not tested |

Early-Eluting Impurities (Method A)

| | | | | | |
|---|---|---|---|---|---|
| iso araguanine | 0.3% | 0.4% | 0.52% | 0.53% | Not tested |
| monophosphate | | | | | |
| iso guanine | ND | ND | 0.01 | 0.02% | Not tested |
| 3,5 diphosphate | ND | ND | 0.01 | 0.02% | Not tested |
| analog | | | | | |
| Any other early- | 0.01% | 0.03% | <0.05% | <0.05% | Not tested |
| eluting impurity | | | | | |

Late-Eluting Impurities (Method B)

| | | | | | |
|---|---|---|---|---|---|
| 2-fluoroadenine | 0.02% | ND | 0.03% | 0.04% | Not tested |
| 2-fluoroara adenine | 0.02% | ND | ND | ND | Not tested |
| 2-Ethoxyphosphate | | ND | ND | ND | Not tested |
| analog | | | | | |
| Any other late- | 0.03% | 0.16% | 0.05% | 0.30% | Not tested |
| eluting impurity | | | (RRT1.03) | (RRT2.07) | |
| | | | 0.46% | 0.25% | |
| | | | RRT2.05) | (RRT2.29) | |
| | | | | 0.08% | |
| | | | | (RRT2.39) | |
| Total Impurities | 0.3% | 0.6% | 0.9% | 1.27% | Not tested |
| (A + B) | | | | | |

ND = Not Detected
RRT = Relative Retention Time

### Example 11. In-Use Stability Assessment of Ready-to-Use Fludarabine Phosphate Compositions in Multi-Dose Vials

Stability of a ready-to-use 30 mg/mL fludarabine phosphate formulation in multi-dose vials was assessed after puncturing the vials with a syringe needle.

Multi-dose vials including ready-to-use 30 mg/mL fludarabine phosphate were prepared consistent with the procedure described in Example 9 and had the formulations shown in Table 22:

TABLE 22
Formulations of Fludarabine Phosphate Compositions
in Multi-Dose Vials for In-Use Study.

| Component | Function | Formula XIIIj | | Formula XIIIk | |
|---|---|---|---|---|---|
| Fludarabine | Active agent | 30 | mg/mL | 30 | mg/mL |
| phosphate, USP | | | | | |
| Mannitol, USP | Tonicity agent | 25 | mg/mL | 0 | mg/mL |
| Sorbitol, USP | Tonicity agent | 0 | mg/mL | 40 | mg/mL |
| Methyl paraben | Preservative | 1.9 | mg/mL | 1.8 | mg/mL |
| Propyl paraben | Preservative | 0.2 | mg/mL | 0.2 | mg/mL |
| Benzyl alcohol | Preservative | 0 | mg/mL | 0 | mg/mL |

| | | | |
|---|---|---|---|
| Sodium | pH adjuster | qs to pH 6.8 | qs to pH 6.8 |
| hydroxide, USP | | | |
| Water for | Solvent | qs to 1 mL | qs to 1 mL |
| injection | | | |
| Nitrogen NF | Inert | qs to fill | qs to fill |
| | atmosphere | headspace | headspace |

The vial seals were then punctured with a syringe needle and the multi-dose vials were then stored at 2-8° C. for 24 hours.

Characteristics of the two test formulations are summarized in Table 23:

TABLE 23
Characteristics of Fludarabine Phosphate Compositions
in Punctured Multi-Dose Vials.

| | Formula XIIIj | | Formula XIIIk | |
|---|---|---|---|---|
| | | 24 hr In- | | 24 hr In- |
| Parameter | Initial | Use Time | Initial | Use Time |
| Description | Clear | Clear | Clear | Clear |
| | colorless | colorless | colorless | colorless |
| | Solution | Solution | Solution | Solution |
| Assay | 96.2% | 95.30% | 97.0% | 96.70% |
| pH | 7.02 | 7.03 | 7.07 | 7.04 |
| Osmolality | NA | NA | NA | NA |

Early-Eluting Impurities (Method A)

| | | | | |
|---|---|---|---|---|
| iso araguanine | 0.24% | 0.24% | 0.24% | 0.24% |
| monophosphate | | | | |
| iso guanine | ND | ND | ND | ND |
| 3,5 diphosphate | ND | ND | ND | ND |
| analog | | | | |
| Any other early- | ND | ND | ND | ND |
| eluting impurity | | | | |

Late-Eluting Impurities (Method B)

| | | | | |
|---|---|---|---|---|
| 2-fluoroadenine | 0.02% | 0.02% | 0.02% | 0.02% |
| 2-fluoroara adenine | ND | ND | ND | ND |
| 2-Ethoxyphosphate | ND | ND | ND | ND |
| analog | | | | |
| Any other late- | 0.03% | 0.02% | 0.04% | 0.03% |
| eluting impurity | | | | |
| Total Impurities | 0.3% | 0.30% | 0.3% | 0.30% |
| (A + B) | | | | |

These data demonstrate that fludarabine phosphate cornpositions consistent with the present disclosure in multi-dose vials remain stable under refrigeration for at least 24 hours after the vial seal is punctured.

### Example 12. Long-Term Stability of Ready-to-Use 30 mg/mL Fludarabine Phosphate Compositions Prepared By Various Methods

Six formulations of 30 mg/mL fludarabine phosphate compositions were prepared by six different methods as follows:

### Formulation XIIIi:

Sorbitol (Merck) was added to water for injection (Merck) that had been purged with nitrogen for 15 minutes. The mixture was then heated to 80° C., at which time methyl paraben (Merck) was added. Propyl paraben (Merck) was added next, and the solution was allowed to cool to ambient temperature. Fludarabine phosphate (Wuhan Biocause) was added and the pH was adjusted to 6.8 with sodium hydroxide (Merck). Additional water for injection (Merck) was added to achieve the final batch volume of 100 mL. Aliquots (5 mL) of this Formulation XIIIi were then placed in vials. Formulation XIIIi had the theoretical characteristics shown in Table 24 below.

TABLE 24
Formula Card (Formulation XIIIi).

| | | | Amount per | | Amount | |
|---|---|---|---|---|---|---|
| Component | Function | Source | 100-mL batch | | per mL | |
| Fludarabine | Active agent | Wuhan | 3000 | mg | 30 | mg |
| phosphate, USP | | Biosource | | | | |
| Sorbitol, USP | Tonicity agent | Merck | 4000 | mg | 40 | mg |
| Methyl paraben, | Preservative | Merck | 180 | mg | 1.8 | mg |
| USP | | | | | | |
| Propyl paraben, | Preservative | Merck | 20 | mg | 0.2 | mg |
| USP | | | | | | |

| Sodium | pH adjuster | Merck | To adjust pH to 6.8 | |
|---|---|---|---|---|
| hydroxide, USP | | | | |
| Water for | Solvent | Merck | q.s. to | q.s. to |
| injection | | | 100 mL | 1 mL |

| | | |
|---|---|---|
| Nitrogen NF | Inert | Inert atmosphere |
| | atmosphere | |

### Formulation XIIIj:

Sorbitol (Merck) was added to water for injection (Merck) and the mixture was then heated to 80° C., at which time methyl paraben (Merck) was added. Propyl paraben (Merck) was added next, and the solution was allowed to cool to ambient temperature. Fludarabine phosphate (Wuhan Biocause) was added and the pH was adjusted to 6.8 with sodium hydroxide (Merck). Additional water for injection (Merck) was added to achieve the final batch volume of 100 mL. Aliquots (5 mL) of this Formulation XIIIj were then placed in vials. Formulation XIIIj had the theoretical characteristics shown in Table 25 below.

TABLE 25
Formula Card (Formulation XIIIj).

| | | | Amount per | | Amount | |
|---|---|---|---|---|---|---|
| Component | Function | Source | 100-mL batch | | per mL | |
| Fludarabine | Active agent | Wuhan | 3000 | mg | 30 | mg |
| phosphate, USP | | Biosource | | | | |
| Sorbitol, USP | Tonicity agent | Merck | 4000 | mg | 40 | mg |
| Methyl paraben, | Preservative | Merck | 180 | mg | 1.8 | mg |
| USP | | | | | | |
| Propyl paraben, | Preservative | Merck | 20 | mg | 0.2 | mg |
| USP | | | | | | |

| Sodium | pH adjuster | Merck | To adjust pH to 6.8 | |
|---|---|---|---|---|
| hydroxide, USP | | | | |
| Water for | Solvent | Merck | q.s. to | q.s. to |
| injection | | | 100 mL | 1 mL |

### Formulation XIIIk:

Sorbitol (Merck) was added to water for injection (Merck) that had been purged with nitrogen for 15 minutes. The mixture was then heated to 8000, at which time methyl paraben (Merck) was added. Propyl paraben (Merck) was added next, and the solution was allowed to cool to ambient temperature. Fludarabine phosphate (Wuhan Biocause) was added and the pH was adjusted to 6.8 with sodium hydroxide (Merck). Additional water for injection (Merck) was added to achieve the final batch volume of 100 mL. Aliquots (5 mL) of this Formulation XIIIk were then placed in vials. Formulation XIIIk had the theoretical characteristics shown in Table 26 below.

TABLE 26
Formula Card (Formulation XIIIk).

| | | | Amount per | | Amount | |
|---|---|---|---|---|---|---|
| Component | Function | Source | 100-mL batch | | per mL | |
| Fludarabine | Active agent | Wuhan | 3000 | mg | 30 | mg |
| phosphate, USP | | Biosource | | | | |
| Sorbitol, USP | Tonicity agent | Merck | 1000 | mg | 10 | mg |
| Methyl paraben, | Preservative | Merck | 180 | mg | 1.8 | mg |
| USP | | | | | | |
| Propyl paraben, | Preservative | Merck | 20 | mg | 0.2 | mg |
| USP | | | | | | |

| Sodium | pH adjuster | Merck | To adjust pH to 6.8 | |
|---|---|---|---|---|
| hydroxide, USP | | | | |
| Water for | Solvent | Merck | q.s. to | q.s. to |
| injection | | | 100 mL | 1 mL |

### Formulation XIIIm:

Sorbitol (Merck) was added to water for injection (Merck) that had been purged with nitrogen for 15 minutes. The mixture was then heated to 80° C., at which time methyl paraben (Merck) was added. Propyl paraben (Merck) was added next, and the solution was allowed to cool to ambient temperature. Fludarabine phosphate (Wuhan Biocause) was added and the pH was adjusted to 6.8 with sodium hydroxide (Merck). Additional water for injection (Merck) was added to achieve the final batch volume of 100 mL. Aliquots (5 mL) of this Formulation XIIIm were then placed in vials. Formulation XIIIm had the theoretical characteristics shown in Table 27 below.

TABLE 27
Formula Card (Formulation XIIIm).

| | | | Amount per | | Amount | |
|---|---|---|---|---|---|---|
| Component | Function | Source | 100-mL batch | | per mL | |
| Fludarabine | Active agent | Wuhan | 3000 | mg | 30 | mg |
| phosphate, USP | | Biosource | | | | |
| Sorbitol, USP | Tonicity agent | Merck | 2000 | mg | 20 | mg |
| Methyl paraben, | Preservative | Merck | 180 | mg | 1.8 | mg |
| USP | | | | | | |
| Propyl paraben, | Preservative | Merck | 20 | mg | 0.2 | mg |
| USP | | | | | | |

| Sodium | pH adjuster | Merck | To adjust pH to 6.8 | |
|---|---|---|---|---|
| hydroxide, USP | | | | |
| Water for | Solvent | Merck | q.s. to | q.s. to |
| injection | | | 100 mL | 1 mL |

### Formulation XIIIn:

Sorbitol (Merck) was added to water for injection (Merck) that had been purged with nitrogen for 15 minutes. The mixture was then heated to 60° C., at which time methyl paraben (Merck) was added. Propyl paraben (Merck) was added next, and the solution was allowed to cool to ambient temperature. Fludarabine phosphate (Wuhan Biocause) was added and the pH was adjusted to 6.8 with sodium hydroxide (Merck). Additional water for injection (Merck) was added to achieve the final batch volume of 100 mL. Aliquots (5 mL) of this Formulation XIIIn were then placed in vials. Formulation XIIIn had the theoretical characteristics shown in Table 28 below.

TABLE 28
Formula Card (Formulation XIIIn).

| | | | Amount per | | Amount | |
|---|---|---|---|---|---|---|
| Component | Function | Source | 100-mL batch | | per mL | |
| Fludarabine | Active agent | Wuhan | 3000 | mg | 30 | mg |
| phosphate, USP | | Biosource | | | | |
| Sorbitol, USP | Tonicity agent | Merck | 4000 | mg | 40 | mg |
| Methyl paraben, | Preservative | Merck | 180 | mg | 1.8 | mg |
| USP | | | | | | |
| Propyl paraben, | Preservative | Merck | 20 | mg | 0.2 | mg |
| USP | | | | | | |

| Sodium | pH adjuster | Merck | To adjust pH to 6.8 | |
|---|---|---|---|---|
| hydroxide, USP | | | | |
| Water for | Solvent | Merck | q.s. to | q.s. to |
| injection | | | 100 mL | 1 mL |

### Formulation XIIIo:

Sorbitol (Merck) was added to water for injection (Merck) that had been purged with nitrogen for 15 minutes. The mixture was then heated to 9000, at which time methyl paraben (Merck) was added. Propyl paraben (Merck) was added next, and the solution was allowed to cool to ambient temperature. Fludarabine phosphate (Wuhan Biocause) was added and the pH was adjusted to 6.8 with sodium hydroxide (Merck). Additional water for injection (Merck) was added to achieve the final batch volume of 100 mL. Aliquots (5 mL) of this Formulation XIIIo were then placed in vials. Formulation XIIIo had the theoretical characteristics shown in Table 29 below.

TABLE 29
Formula Card (Formulation XIIIo).

| | | | Amount per | | Amount | |
|---|---|---|---|---|---|---|
| Component | Function | Source | 100-mL batch | | per mL | |
| Fludarabine | Active agent | Wuhan | 3000 | mg | 30 | mg |
| phosphate, USP | | Biosource | | | | |
| Sorbitol, USP | Tonicity agent | Merck | 4000 | mg | 40 | mg |
| Methyl paraben, | Preservative | Merck | 180 | mg | 1.8 | mg |
| USP | | | | | | |
| Propyl paraben, | Preservative | Merck | 20 | mg | 0.2 | mg |
| USP | | | | | | |

| Sodium | pH adjuster | Merck | To adjust pH to 6.8 | |
|---|---|---|---|---|
| hydroxide, USP | | | | |
| Water for | Solvent | Merck | q.s. to | q.s. to |
| injection | | | 100 mL | 1 mL |

### Example 13. Stability Assessment of Ready-to-Use 30 mg/mL Fludarabine Phosphate Compositions

Stability of a variety of ready-to-use compositions of the ready-to-use fludarabine phosphate compositions in multi-dose vials of Example 12 (i.e., Formulations XIIIi to XIIIo) was assessed under refrigerated, real-time, and accelerated storage conditions.

Briefly, the vials prepared in Example 12 were stored at 2-8° C., at 25° C. and 60% relative humidity, or at 40° C. and 75% relative humidity. Characteristics of each stored formulation before storage (T0), after two weeks (T=2W; samples stored at 25° C./60% RH or at 40° C./75% RH only), after one month (T=1 M; samples stored at 2-8° C., or at 25° C./60% RH only), and after three months (3M samples stored at 2-8° C. or at 25° C./60% RH only) are shown in Tables 30-35, respectively:

TABLE 30
Characteristics of Formula XIIIi After Storage.

| 2-8° C. | T0 | T = 2 W | T = 1 M | T = 3 M |
|---|---|---|---|---|
| Description | Clear | Not tested | Clear | Clear |
| | colorless | | colorless | colorless |
| | solution | | solution | solution |
| Assay | 102.2% | Not tested | 100% | 102.7% |
| pH | 6.42 | Not tested | 6.8 | 6.8 |
| Osmolality | 250 | Not tested | Not tested | Not tested |
| iso araguanine | 0.28% | Not tested | 0.2% | 0.2% |
| monophosphate | | | | |
| iso guanine | ND | Not tested | 0.01% | ND |
| 3,5 diphosphate | ND | Not tested | ND | ND |
| analog | | | | |
| Any other early- | ND | Not tested | ND | ND |
| eluting impurity | | | | |
| 2-fluoroadenine | ND | Not tested | 0.02% | ND |
| 2-fluoroara adenine | ND | Not tested | ND | ND |
| 2-Ethoxyphosphate | ND | Not tested | ND | ND |
| analog | | | | |
| Any other late- | 0.05% | Not tested | 0.05% | 0.1% |
| eluting impurity | | | | |
| Total Impurities | 0.3% | Not tested | 0.3% | 0.3% |
| (A + B) | | | | |
| 25° C./60RH | T0 | T = 2 W | T = 1 M | T = 3 M |
| Description | Clear | Clear | Clear | Clear |
| | colorless | colorless | colorless | colorless |
| | solution | solution | solution | solution |
| Assay | 102.2% | 101.6% | 101% | 101.1% |
| pH | 6.42 | 6.7 | 6.7 | 6.8 |
| Osmolality | 250 | Not tested | Not tested | Not tested |
| iso araguanine | 0.28% | 0.33% | 0.3% | 0.4% |
| monophosphate | | | | |
| iso guanine | ND | ND | 0.01% | ND |
| 3,5 diphosphate | ND | ND | ND | ND |
| analog | | | | |
| Any other early- | ND | ND | ND | <0.05% |
| eluting impurity | | | | |
| 2-fluoroadenine | ND | 0.03% | 0.03% | 0.03% |
| 2-fluoroara | ND | 0.02% | ND | ND |
| adenine | | | | |
| 2-Ethoxyphosphate | ND | ND | ND | ND |
| analog | | | | |
| Any other late- | 0.05% | 0.05% | 0.07% | 0.3% |
| eluting impurity | | | | |
| Total Impurities | 0.3% | 0.4% | 0.4% | 0.7% |
| (A + B) | | | | |

| 40° C./75RH | T0 | T = 2 W |
|---|---|---|
| Description | Clear | Clear |
| | colorless | colorless |
| | solution | solution |
| Assay | 102.2% | 101.6% |
| pH | 6.42 | 6.7 |
| Osmolality | 250 | Not Tested |
| iso araguanine | 0.28% | 0.34% |
| monophosphate | | |
| iso guanine | ND | 0.03% |
| 3,5 diphosphate | ND | ND |
| analog | | |
| Any other early- | ND | ND |
| eluting impurity | | |
| 2-fluoroadenine | ND | ND |
| 2-fluoroara adenine | ND | 0.07% |
| 2-Ethoxyphosphate | ND | ND |
| analog | | |
| Any other late- | 0.05% | 0.2% |
| eluting impurity | | |
| Total Impurities | 0.3% | 0.7% |
| (A + B) | | |

ND = Not Detected
RRT = Relative Retention Time

TABLE 31
Characteristics of Formula XIIIj After Storage.

| 2-8° C. | T0 | T = 2 W | T = 1 M | T = 3 M |
|---|---|---|---|---|
| Description | Clear | Not tested | Clear | Clear |
| | colorless | | colorless | colorless |
| | solution | | solution | solution |
| Assay | 100.9% | Not tested | 100.5% | 101.9% |
| pH | 6.64 | Not tested | 6.7 | 6.7 |
| Osmolality | 326 | Not tested | Not tested | Not tested |
| iso araguanine | 0.28% | Not tested | 0.2% | 0.2% |
| monophosphate | | | | |
| iso guanine | ND | Not tested | 0.01% | ND |
| 3,5 diphosphate | ND | Not tested | ND | ND |
| analog | | | | |
| Any other early- | ND | Not tested | ND | ND |
| eluting impurity | | | | |
| 2-fluoroadenine | ND | Not tested | 0.02% | ND |
| 2-fluoroara adenine | ND | Not tested | ND | ND |
| 2-Ethoxyphosphate | ND | Not tested | ND | ND |
| analog | | | | |
| Any other late- | 0.05% | Not tested | 0.05% | 0.1% |
| eluting impurity | | | | |
| Total Impurities | 0.3% | Not tested | 0.3% | 0.3% |
| (A + B) | | | | |
| 25° C./60RH | T0 | T = 2 W | T = 1 M | T = 3 M |
| Description | Clear | Clear | Clear | Clear |
| | colorless | colorless | colorless | colorless |
| | solution | solution | solution | solution |
| Assay | 100.9% | 101.5% | 100.9% | 100.7% |
| pH | 6.64 | 6.7 | 6.6 | 6.7 |
| Osmolality | 326 | Not tested | Not tested | Not tested |
| iso araguanine | 0.28% | 0.3% | 0.3% | 0.4% |
| monophosphate | | | | |
| iso guanine | ND | ND | 0.01% | <0.05% |
| 3,5 diphosphate | ND | ND | ND | ND |
| analog | | | | |
| Any other early- | ND | ND | ND | ND |
| eluting impurity | | | | |
| 2-fluoroadenine | ND | 0.03% | 0.02% | 0.02% |
| 2-fluoroara adenine | ND | 0.02% | <0.05% | ND |
| 2-Ethoxyphosphate | ND | <0.05% | ND | ND |
| analog | | | | |
| Any other late- | 0.05% | 0.05% | 0.07% | 0.30% |
| eluting impurity | | | | |
| Total Impurities | 0.3% | 0.4% | 0.4% | 0.6% |
| (A + B) | | | | |

| 40° C./75RH | T0 | T = 2 W |
|---|---|---|
| Description | Clear | Clear |
| | colorless | colorless |
| | solution | solution |
| Assay | 100.9% | 101.5% |
| pH | 6.64 | 6.6 |
| Osmolality | 326 | Not tested |
| iso araguanine | 0.28% | 0.4% |
| monophosphate | | |
| iso guanine | ND | 0.03% |
| 3,5 diphosphate | ND | ND |
| analog | | |
| Any other early- | ND | ND |
| eluting impurity | | |
| 2-fluoroadenine | ND | ND |
| 2-fluoroara adenine | ND | 0.06% |
| 2-Ethoxyphosphate | ND | ND |
| analog | | |
| Any other late- | 0.05% | 0.2% |
| eluting impurity | | |
| Total Impurities | 0.3% | 0.7% |
| (A + B) | | |

ND = Not Detected
RRT = Relative Retention Time

TABLE 32
Characteristics of Formula XIIIk After Storage.

| 2-8° C. | T0 | T = 2 W | T = 1 M | T = 3 M |
|---|---|---|---|---|
| Description | Clear | Not tested | Clear | Clear |
| | colorless | | colorless | colorless |
| | solution | | solution | solution |
| Assay | 102.7% | Not tested | 100.1% | 103.7% |
| pH | 6.64 | Not tested | 6.7 | 6.7 |
| Osmolality | 216 | Not tested | Not tested | Not tested |
| iso araguanine | 0.28% | Not tested | 0.2% | 0.2% |
| monophosphate | | | | |
| iso guanine | ND | Not tested | 0.01% | ND |
| 3,5 diphosphate | ND | Not tested | ND | ND |
| analog | | | | |
| Any other early- | ND | Not tested | ND | ND |
| eluting impurity | | | | |
| 2-fluoroadenine | ND | Not tested | 0.02% | ND |
| 2-fluoroara adenine | ND | Not tested | ND | ND |
| 2-Ethoxyphosphate | ND | Not tested | ND | ND |
| analog | | | | |
| Any other late- | ND | Not tested | 0.05% | 0.1% |
| eluting impurity | | | | |
| Total Impurities | 0.3% | Not tested | 0.3% | 0.3% |
| (A + B) | | | | |
| 25° C./60RH | T0 | T = 2 W | T = 1 M | T = 3 M |
| Description | Clear | Clear | Clear | Clear |
| | colorless | colorless | colorless | colorless |
| | solution | solution | solution | solution |
| Assay | 102.7% | 101.4% | 102.2% | 101.9% |
| pH | 6.64 | 6.6 | 6.7 | 6.7 |
| Osmolality | 216 | Not tested | Not tested | Not tested |
| iso araguanine | 0.3% | 0.3% | 0.3% | 0.4% |
| monophosphate | | | | |
| iso guanine | ND | 0.03% | 0.01% | ND |
| 3,5 diphosphate | <0.05% | ND | ND | ND |
| analog | | | | |
| Any other early- | <0.05% | ND | ND | ND |
| eluting impurity | | | | |
| 2-fluoroadenine | <0.05% | 0.03% | 0.02% | 0.02% |
| 2-fluoroara adenine | <0.05% | 0.02% | <0.05% | ND |
| 2-Ethoxyphosphate | ND | ND | ND | ND |
| analog | | | | |
| Any other late- | <0.05% | 0.05% | 0.07% | 0.3% |
| eluting impurity | | | | |
| Total Impurities | 0.3% | 0.3% | 0.4% | 0.6% |
| (A + B) | | | | |

| 40° C./75RH | T0 | T = 2 W |
|---|---|---|
| Description | Clear | Clear |
| | colorless | colorless |
| | solution | solution |
| Assay | 102.7% | 101.5% |
| pH | 6.64 | 6.6 |
| Osmolality | 216 | Not tested |
| iso araguanine | 0.28% | 0.3% |
| monophosphate | | |
| iso guanine | ND | 0.03% |
| 3,5 diphosphate | ND | ND |
| analog | | |
| Any other early- | ND | ND |
| eluting impurity | | |
| 2-fluoroadenine | ND | ND |
| 2-fluoroara adenine | ND | 0.06% |
| 2-Ethoxyphosphate | ND | ND |
| analog | | |
| Any other late- | ND | 0.2% |
| eluting impurity | | |
| Total Impurities | 0.3% | 0.7% |
| (A + B) | | |

ND = Not Detected
RRT = Relative Retention Time

TABLE 33
Characteristics of Formula XIIIm After Storage.

| 2-8° C. | T0 | T = 2 W | T = 1 M | T = 3 M |
|---|---|---|---|---|
| Description | Clear | Not tested | Clear | Clear |
| | colorless | | colorless | colorless |
| | solution | | solution | solution |
| Assay | 100.5% | Not tested | 100.9% | 102.1% |
| pH | 6.65 | Not tested | 6.8 | 6.8 |
| Osmolality | 321 | Not tested | Not tested | Not tested |
| iso araguanine | 0.28% | Not tested | 0.2% | 0.2% |
| monophosphate | | | | |
| iso guanine | ND | Not tested | 0.01% | ND |
| 3,5 diphosphate | ND | Not tested | ND | ND |
| analog | | | | |
| Any other early- | ND | Not tested | ND | ND |
| eluting impurity | | | | |
| 2-fluoroadenine | ND | Not tested | 0.02% | ND |
| 2-fluoroara adenine | ND | Not tested | ND | ND |
| 2-Ethoxyphosphate | ND | Not tested | ND | ND |
| analog | | | | |
| Any other late- | ND | Not tested | 0.05% | 0.1% |
| eluting impurity | | | | |
| Total Impurities | 0.3% | Not tested | 0.3% | 0.3% |
| (A + B) | | | | |
| 25° C./60RH | T0 | T = 2 W | T = 1 M | T = 3 M |
| Description | Clear | Clear | Clear | Clear |
| | colorless | colorless | colorless | colorless |
| | solution | solution | solution | solution |
| Assay | 100.5% | 101.4% | 99.9% | 99.1% |
| pH | 6.65 | 6.59 | 6.8 | 6.8 |
| Osmolality | 321 | Not tested | Not tested | Not tested |
| iso araguanine | 0.28% | 0.32% | 0.3% | 0.4% |
| monophosphate | | | | |
| iso guanine | ND | 0.01% | 0.01% | ND |
| 3,5 diphosphate | ND | ND | <0.05% | ND |
| analog | | | | |
| Any other early- | ND | ND | ND | ND |
| eluting impurity | | | | |
| 2-fluoroadenine | ND | 0.03% | 0.024% | 0.03% |
| 2-fluoroara adenine | ND | 0.02% | ND | ND |
| 2-Ethoxyphosphate | ND | ND | ND | ND |
| analog | | | | |
| Any other late- | ND | 0.08% | 0.13% | 0.4% |
| eluting impurity | | | | |
| Total Impurities | 0.3% | 0.4% | 0.5% | 0.8% |
| (A + B) | | | | |

| 40° C./75RH | T0 | T = 2 W |
|---|---|---|
| Description | Clear | Clear |
| | colorless | colorless |
| | solution | solution |
| Assay | 100.5% | 101.6% |
| pH | 6.65 | 6.6 |
| Osmolality | 321 | Not tested |
| iso araguanine | 0.28% | 0.3% |
| monophosphate | | |
| iso guanine | ND | 0.02% |
| 3,5 diphosphate | ND | ND |
| analog | | |
| Any other early- | ND | ND |
| eluting impurity | | |
| 2-fluoroadenine | ND | ND |
| 2-fluoroara adenine | ND | 0.08% |
| 2-Ethoxyphosphate | ND | ND |
| analog | | |
| Any other late- | ND | 0.2% |
| eluting impurity | | |
| Total Impurities | 0.3% | 0.7% |
| (A + B) | | |

ND = Not Detected
RRT = Relative Retention Time

TABLE 34
Characteristics of Formula XIIIn After Storage.

| 2-8° C. | T0 | T = 2 W | T = 1 M | T = 3 M |
|---|---|---|---|---|
| Description | Clear | Not tested | Clear | Clear |
| | colorless | | colorless | colorless |
| | solution | | solution | solution |
| Assay | 100.7% | Not tested | 100.9% | 102.1% |
| pH | 6.55 | Not tested | 6.7 | 6.7 |
| Osmolality | 316 | Not tested | Not tested | Not tested |
| iso araguanine | 0.35% | Not tested | 0.3% | 0.2% |
| monophosphate | | | | |
| iso guanine | ND | Not tested | 0.02% | ND |
| 3,5 diphosphate | ND | Not tested | ND | ND |
| analog | | | | |
| Any other early- | ND | Not tested | ND | ND |
| eluting impurity | | | | |
| 2-fluoroadenine | ND | Not tested | 0.02% | ND |
| 2-fluoroara adenine | ND | Not tested | ND | ND |
| 2-Ethoxyphosphate | ND | Not tested | ND | ND |
| analog | | | | |
| Any other late- | ND | Not tested | 0.05% | 0.1% |
| eluting impurity | | | | |
| Total Impurities | 0.4% | Not tested | 0.4% | 0.4% |
| (A + B) | | | | |
| 25° C./60RH | T0 | T = 2 W | T = 1 M | T = 3 M |
| Description | Clear | Clear | Clear | Clear |
| | colorless | colorless | colorless | colorless |
| | solution | solution | solution | solution |
| Assay | 100.7% | 101.4% | 100.4% | 99% |
| pH | 6.55 | 6.6 | 6.7 | 6.8 |
| Osmolality | 316 | Not tested | Not tested | Not tested |
| iso araguanine | 0.35% | 0.3% | 0.3% | 0.5% |
| monophosphate | | | | |
| iso guanine | ND | 0.01% | 0.02% | ND |
| 3,5 diphosphate | ND | ND | ND | ND |
| analog | | | | |
| Any other early- | ND | ND | ND | ND |
| eluting impurity | | | | |
| 2-fluoroadenine | ND | 0.03% | 0.03% | 0.03% |
| 2-fluoroara adenine | ND | 0.02% | ND | ND |
| 2-Ethoxyphosphate | ND | ND | ND | ND |
| analog | | | | |
| Any other late- | ND | 0.08% | 0.09% | 0.3% |
| eluting impurity | | | | |
| Total Impurities | 0.4% | 0.4% | 0.5% | 0.8% |
| (A + B) | | | | |

| 40° C./75RH | T0 | T = 2 W |
|---|---|---|
| Description | Clear | Clear |
| | colorless | colorless |
| | solution | solution |
| Assay | 100.7% | 101.5% |
| pH | 6.55 | 6.7 |
| Osmolality | 316 | Not tested |
| iso araguanine | 0.35% | 0.4% |
| monophosphate | | |
| iso guanine | ND | 0.02% |
| 3,5 diphosphate | ND | ND |
| analog | | |
| Any other early- | ND | ND |
| eluting impurity | | |
| 2-fluoroadenine | ND | ND |
| 2-fluoroara adenine | ND | 0.07% |
| 2-Ethoxyphosphate | ND | ND |
| analog | | |
| Any other late- | ND | 0.2% |
| eluting impurity | | |
| Total Impurities | 0.3% | 0.7% |
| (A + B) | | |

ND = Not Detected
RRT = Relative Retention Time

TABLE 35
Characteristics of Formula XIIIo After Storage.

| 2-8° C. | T0 | T = 2 W | T = 1 M | T = 3 M |
|---|---|---|---|---|
| Description | Clear | Not tested | Clear | Clear |
| | colorless | | colorless | colorless |
| | solution | | solution | solution |
| Assay | 101% | Not tested | 99.8% | 101.8% |
| pH | 6.48 | Not tested | 6.7 | 6.7 |
| Osmolality | 315 | Not tested | Not tested | Not tested |
| iso araguanine | 0.34% | Not tested | 0.3% | 0.3% |
| monophosphate | | | | |
| iso guanine | ND | Not tested | 0.01% | ND |
| 3,5 diphosphate | ND | Not tested | ND | ND |
| analog | | | | |
| Any other early- | ND | Not tested | ND | ND |
| eluting impurity | | | | |
| 2-fluoroadenine | ND | Not tested | 0.03% | ND |
| 2-fluoroara adenine | ND | Not tested | ND | ND |
| 2-Ethoxyphosphate | ND | Not tested | ND | ND |
| analog | | | | |
| Any other late- | 0.05% | Not tested | 0.05% | 0.1% |
| eluting impurity | | | | |
| Total Impurities | 0.4% | Not tested | 0.3% | 0.4% |
| (A + B) | | | | |
| 25° C./60RH | T0 | T = 2 W | T = 1 M | T = 3 M |
| Description | Clear | Clear | Clear | Clear |
| | colorless | colorless | colorless | colorless |
| | solution | solution | solution | solution |
| Assay | 101% | 101.4% | 99.9% | 100.6% |
| pH | 6.46 | 6.61 | 6.7 | 6.7 |
| Osmolality | 315 | Not tested | Not tested | Not tested |
| iso araguanine | 0.34% | 0.35% | 0.3% | 0.4% |
| monophosphate | | | | |
| iso guanine | ND | ND | 0.02% | ND |
| 3,5 diphosphate | ND | ND | ND | ND |
| analog | | | | |
| Any other early- | ND | ND | ND | ND |
| eluting impurity | | | | |
| 2-fluoroadenine | ND | 0.03% | 0.03% | 0.03% |
| 2-fluoroara adenine | ND | 0.02% | ND | ND |
| 2-Ethoxyphosphate | ND | ND | ND | ND |
| analog | | | | |
| Any other late- | 0.05% | 0.06% | 0.08% | 0.3% |
| eluting impurity | | | | |
| Total Impurities | 0.4% | 0.41% | 0.5% | 0.7% |
| (A + B) | | | | |

| 40° C./75RH | T0 | T = 2 W |
|---|---|---|
| Description | Clear | Clear |
| | colorless | colorless |
| | solution | solution |
| Assay | 101% | 101.5% |
| pH | 6.46 | 6.7 |
| Osmolality | 315 | Not tested |
| iso araguanine | 0.3% | 0.4% |
| monophosphate | | |
| iso guanine | ND | 0.02% |
| 3,5 diphosphate | ND | ND |
| analog | | |
| Any other early- | ND | ND |
| eluting impurity | | |
| 2-fluoroadenine | ND | ND |
| 2-fluoroara adenine | ND | 0.07% |
| 2-Ethoxyphosphate | ND | ND |
| analog | | |
| Any other late- | 0.05% | 0.2% |
| eluting impurity | | |
| Total Impurities | 0.4% | 0.8% |
| (A + B) | | |

ND = Not Detected
RRT = Relative Retention Time

### FURTHER EXAMPLES

Further Example 1. A method of treating a lymphoma or a leukemia in a subject in need thereof, the method comprising:
lymphodepleting the subject by administering an amount of fludarabine not exceeding about 25 mg/m²; and
thereafter administering CAR-T cells to the subject.

Further Example 2. The method of Further Example 1, wherein the fludarabine comprises, consists essentially of, or consists of fludarabine phosphate.

Further Example 3. The method of Further Example 1 or Further Example 2, wherein the step of lymphodepleting comprises:
withdrawing a sterile premixed solution comprising not more than about 25 mg/mL fludarabine phosphate from a container into a syringe; and
injecting the solution into the subject to provide an amount of fludarabine phosphate not exceeding about 25 mg/m².

Further Example 4. The method of Further Example 3, wherein the method does not include mixing contents of the container with water before the step of withdrawing the sterile premixed solution into the syringe.

Further Example 5. The method of any one preceding Further Example, wherein the sterile premixed solution is prepared by a process comprising:
mixing fludarabine phosphate, mannitol, an optional pH adjuster and water to form a bulk solution having an initial pH level of about 6.5 to about 7.1;
filtering the bulk solution through a sterilization grade filter to form a sterile bulk solution;
filling one or more containers with at least a portion of the sterile bulk solution; and
sealing the one or more containers.

Further Example 6. The method of Further Example 5, wherein the sterilization grade filter has a pore size of about 0.2 microns.

Further Example 7. The method of Further Example 5 or Further Example 6, wherein the process of preparing the sterile premixed solution further comprises sterilizing the one or more containers before the step of filling the one or more containers.

Further Example 8. The method of any one preceding Further Example, wherein the sterile premixed solution comprises:
not more than 25 mg/mL fludarabine phosphate;
a tonicity agent;
water; and
an initial pH level of about 6.5 to about 7.1.

Further Example 9. The method of Further Example 8, wherein the sterile premixed solution consists essentially of:
not more than 25 mg/mL fludarabine phosphate;
a tonicity agent;
water; and
an initial pH level of about 6.5 to about 7.1.

Further Example 10. The method of Further Example 8, wherein the sterile premixed solution consists of:
not more than 25 mg/mL fludarabine phosphate;
a tonicity agent;
water; and
an initial pH level of about 6.5 to about 7.1.

Further Example 11. The method of any one of Further Examples 8-10, wherein the sterile premixed solution has an initial pH level of about 6.6 to about 7.0.

Further Example 12. The method of any one of Further Examples 8-10, wherein the sterile premixed solution has an initial pH level of about 6.8.

Further Example 13. The method of any one of Further Examples 8-12 wherein, after storage at about 5° C. for 3 months, the sterile premixed solution has a pH level within 0.5 pH units of the initial pH level.

Further Example 14. The method of any one of Further Examples 8-13 wherein, after storage at about 5° C. for 6 months, the sterile premixed solution has a pH level within 0.5 pH units of the initial pH level.

Further Example 15. The method of any one of Further Examples 8-14 wherein, after storage at about 5° C. for 9 months, the sterile premixed solution has a pH level within 0.5 pH units of the initial pH level.

Further Example 16. The method of any one of Further Examples 8-15 wherein, after storage at about 5° C. for 18 months, the sterile premixed solution has a pH level within 0.5 pH units of the initial pH level.

Further Example 17. The method of any one of Further Examples 8-16 wherein, after storage at about 5° C. for 24 months, the sterile premixed solution has a pH level within 0.5 pH units of the initial pH level.

Further Example 18. The method of any one of Further Examples 8-12 wherein, after storage at about 25° C. and about 60% relative humidity (RH) for 1 month, the sterile premixed solution has a pH level within 0.5 pH units of the initial pH level.

Further Example 19. The method of any one of Further Examples 8-13 wherein, after storage at about 25° C. and about 60% relative humidity (RH) for 2 months, the sterile premixed solution has a pH level within 0.5 pH units of the initial pH level.

Further Example 20. The method of any one of Further Examples 8-14 wherein, after storage at about 25° C. and about 60% relative humidity (RH) for 3 months, the sterile premixed solution has a pH level within 0.5 pH units of the initial pH level.

Further Example 21. The method of any one preceding Further Example, wherein the step of administering the sterile premixed solution comprises injecting the solution through skin of the subject.

Further Example 22. The method of any one preceding Further Example, wherein the step of lymphodepleting further comprises administering cyclophosphamide to the subject.

Further Example 23. The method of Further Example 22, wherein the cyclophosphamide is administered in an amount less than 300 mg/m².

Further Example 24. The method of Further Example 22, wherein the cyclophosphamide is administered in an amount of about 300 mg/m².

Further Example 25. The method of any one preceding Further Example wherein, after storage at about 5° C. for 12 months or 24 months, the sterile premixed solution has not more than 2% total impurities.

Further Example 26. The method of any one preceding Further Example wherein, after storage at about 5° C. for 12 months or 24 months, the sterile premixed solution has not more than 1% iso-ara-guanine monophosphate impurity.

Further Example 27. The method of any one preceding Further Example wherein, after storage at about 5° C. for 12 months or 24 months, the sterile premixed solution has not more than 0.2% isoguanine impurity.

Further Example 28. The method of any one preceding Further Example wherein, after storage at about 5° C. for 12 months or 24 months, the sterile premixed solution has not more than 0.2% of any impurity other than iso-ara-guanine monophosphate or isoguanine.

Further Example 29. The method of any one preceding Further Example wherein, after storage at about 5° C. for 12 months or 24 months, the sterile premixed solution has not more than 0.2% 2-fluoroadenine impurity.

Further Example 30. The method of any one preceding Further Example wherein, after storage at about 5° C. for 12 months or 24 months, the sterile premixed solution has not more than 0.2% 2-fluoro-ara-adenine impurity.

Further Example 31. A method of lymphodepleting a subject, the method comprising administering to the subject an amount of fludarabine not exceeding about 25 mg/m².

Further Example 32. The method of Further Example 31, wherein the fludarabine comprises, consists essentially of, or consists of fludarabine phosphate.

Further Example 33. The method of Further Example 31 or Further Example 32, wherein the step of lymphodepleting comprises:
withdrawing a sterile premixed solution comprising not more than about 25 mg/mL fludarabine phosphate from a container into a syringe; and
injecting the solution into the subject to provide an amount of fludarabine phosphate not exceeding about 25 mg/m².

Further Example 34. The method of Further Example 33, wherein the method does not include mixing contents of the container with water before the step of withdrawing the sterile premixed solution into the syringe.

Further Example 35. The method of Further Example 33 or Further Example 34, wherein the sterile premixed solution is prepared by a process comprising:
mixing fludarabine phosphate, mannitol, an optional pH adjuster and water to form a bulk solution having an initial pH level of about 6.5 to about 7.1;
filtering the bulk solution through a sterilization grade filter to form a sterile bulk solution;
filling one or more containers with at least a portion of the sterile bulk solution; and
sealing the one or more containers.

Further Example 36. The method of Further Example 35, wherein the sterilization grade filter has a pore size of about 0.2 microns.

Further Example 37. The method of Further Example 35 or Further Example 6, wherein the process of preparing the sterile premixed solution further comprises sterilizing the one or more containers before the step of filling the one or more containers.

Further Example 38. The method of any one of Further Examples 33-37, wherein the sterile premixed solution comprises:
not more than 25 mg/mL fludarabine phosphate;
a tonicity agent;
water; and
an initial pH level of about 6.5 to about 7.1.

Further Example 39. The method of Further Example 38, wherein the sterile premixed solution consists essentially of:
not more than 25 mg/mL fludarabine phosphate;
a tonicity agent;
water; and
an initial pH level of about 6.5 to about 7.1.

Further Example 40. The method of Further Example 38, wherein the sterile premixed solution consists of:
not more than 25 mg/mL fludarabine phosphate;
a tonicity agent;
water; and
an initial pH level of about 6.5 to about 7.1.

Further Example 41. The method of any one of Further Examples 38-40, wherein the sterile premixed solution has an initial pH level of about 6.6 to about 7.0.

Further Example 42. The method of any one of Further Examples 38-40, wherein the sterile premixed solution has an initial pH level of about 6.8.

Further Example 43. The method of any one of Further Examples 38-42 wherein, after storage at about 5° C. for 3 months, the sterile premixed solution has a pH level within 0.5 pH units of the initial pH level.

Further Example 44. The method of any one of Further Examples 38-43 wherein, after storage at about 5° C. for 6 months, the sterile premixed solution has a pH level within 0.5 pH units of the initial pH level.

Further Example 45. The method of any one of Further Examples 38-44 wherein, after storage at about 5° C. for 9 months, the sterile premixed solution has a pH level within 0.5 pH units of the initial pH level.

Further Example 46. The method of any one of Further Examples 38-45 wherein, after storage at about 5° C. for 18 months, the sterile premixed solution has a pH level within 0.5 pH units of the initial pH level.

Further Example 47. The method of any one of Further Examples 38-46 wherein, after storage at about 5° C. for 24 months, the sterile premixed solution has a pH level within 0.5 pH units of the initial pH level.

Further Example 48. The method of any one of Further Examples 38-42 wherein, after storage at about 25° C. and about 60% relative humidity (RH) for 1 month, the sterile premixed solution has a pH level within 0.5 pH units of the initial pH level.

Further Example 49. The method of any one of Further Examples 38-43 wherein, after storage at about 25° C. and about 60% relative humidity (RH) for 2 months, the sterile premixed solution has a pH level within 0.5 pH units of the initial pH level.

Further Example 50. The method of any one of Further Examples 38-44 wherein, after storage at about 25° C. and about 60% relative humidity (RH) for 3 months, the sterile premixed solution has a pH level within 0.5 pH units of the initial pH level.

Further Example 51. The method of any one of Further Examples 31-50, wherein the step of lymphodepleting further comprises administering cyclophosphamide to the subject.

Further Example 52. The method of Further Example 51, wherein the cyclophosphamide is administered in an amount less than 300 mg/m².

Further Example 53. The method of Further Example 51, wherein the cyclophosphamide is administered in an amount of about 300 mg/m².

Further Example 54. The method of any one of Further Examples 31-53 further comprising administering CAR-T cells to the subject after the step of administering the fludarabine to the subject.

Further Example 55. The method of Further Example 54 further comprising collecting T cells from the subject before the step of administering the fludarabine to the subject.

Further Example 56. The method of any one of Further Examples 31-55 wherein, after storage at about 5° C. for 12 months or 24 months, the sterile premixed solution has not more than 2% total impurities.

Further Example 57. The method of any one of Further Examples 31-56 wherein, after storage at about 5° C. for 12 months or 24 months, the sterile premixed solution has not more than 1% iso-ara-guanine monophosphate impurity.

Further Example 58. The method of any one of Further Examples 31-57 wherein, after storage at about 5° C. for 12 months or 24 months, the sterile premixed solution has not more than 0.2% isoguanine impurity.

Further Example 59. The method of any one of Further Examples 31-58 wherein, after storage at about 5° C. for 12 months or 24 months, the sterile premixed solution has not more than 0.2% of any impurity other than iso-ara-guanine monophosphate or isoguanine.

Further Example 60. The method of any one of Further Examples 31-59 wherein, after storage at about 5° C. for 12 months or 24 months, the sterile premixed solution has not more than 0.2% 2-fluoroadenine impurity.

Further Example 61. The method of any one of Further Examples 31-60 wherein, after storage at about 5° C. for 12 months or 24 months, the sterile premixed solution has not more than 0.2% 2-fluoro-ara-adenine impurity.

What is claimed is:

1. A pharmaceutical product comprising:
a multi-use container; and
a sterile composition housed within the multi-use container, the composition including:
fludarabine phosphate in an amount selected from the group consisting of: 25 mg/mL and 30 mg/mL,
mannitol in an amount selected from the group consisting of: 0 mg/mL, 25 mg/mL and 40 mg/mL,
sorbitol in an amount selected from the group consisting of: 0 mg/mL and 40 mg/mL,
methyl paraben in an amount selected from the group consisting of: 0 mg/mL and 1.8 mg/mL,
propyl paraben in an amount selected from the group consisting of: 0 mg/mL and 0.2 mg/mL,
benzyl alcohol in an amount selected from the group consisting of: 0 mg/mL and 25 mg/mL,
water for injection,
an optional pH adjuster,
an inert gaseous atmosphere, and
an initial pH level of 6.5 to 7.1.

2. The pharmaceutical product of claim 1, wherein the composition includes:
0 mg/mL methyl paraben;
0 mg/mL propyl paraben; and
0 mg/mL benzyl alcohol.

3. The pharmaceutical product of claim 2, wherein the composition includes 0 mg/mL sorbitol.

4. The pharmaceutical product of claim 1, wherein the composition includes:
25 mg/mL fludarabine phosphate;
25 mg/mL mannitol;
0 mg/mL sorbitol;
1.8 mg/mL methyl paraben;
0.2 mg/mL propyl paraben; and
0 mg/mL benzyl alcohol.

5. The pharmaceutical product of claim 1, wherein the composition includes:
25 mg/mL fludarabine phosphate;
0 mg/mL mannitol;
40 mg/mL sorbitol;
1.8 mg/mL methyl paraben;
0.2 mg/mL propyl paraben; and
0 mg/mL benzyl alcohol.

6. The pharmaceutical product of claim 1, wherein the composition includes:
25 mg/mL fludarabine phosphate;
25 mg/mL mannitol;
0 mg/mL sorbitol;
0 mg/mL methyl paraben;
0 mg/mL propyl paraben; and
25 mg/mL benzyl alcohol.

7. The pharmaceutical product of claim 1, wherein the composition includes:
30 mg/mL fludarabine phosphate;
40 mg/mL mannitol;
0 mg/mL sorbitol;
0 mg/mL methyl paraben;
0 mg/mL propyl paraben; and
0 mg/mL benzyl alcohol.

8. The pharmaceutical product of claim 1, wherein the composition includes:
30 mg/mL fludarabine phosphate;
0 mg/mL mannitol;
40 mg/mL sorbitol;
0 mg/mL methyl paraben;
0 mg/mL propyl paraben; and
0 mg/mL benzyl alcohol.

9. The pharmaceutical product of claim 1, wherein the composition includes:
30 mg/mL fludarabine phosphate;
25 mg/mL mannitol;
0 mg/mL sorbitol;
0 mg/mL methyl paraben;
0 mg/mL propyl paraben; and
25 mg/mL benzyl alcohol.

10. The pharmaceutical product of claim 1, wherein the composition includes:
30 mg/mL fludarabine phosphate;
25 mg/mL mannitol;
0 mg/mL sorbitol;
1.8 mg/mL methyl paraben;
0.2 mg/mL propyl paraben; and
0 mg/mL benzyl alcohol.

11. The pharmaceutical product of claim 1, wherein the composition includes:
30 mg/mL fludarabine phosphate;
0 mg/mL mannitol;
40 mg/mL sorbitol;
1.8 mg/mL methyl paraben;
0.2 mg/mL propyl paraben; and
0 mg/mL benzyl alcohol.

12. The pharmaceutical product of claim 1, wherein after storage at 5° C.+/−3° C. for at least 12 months, the composition includes:
not more than 1.0% iso-ara-guanine-monophosphate;
not more than 0.2% isoguanine;
not more than 0.2% of any other early-eluting impurity;
not more than 0.2% 2-fluoroadenine;
not more than 0.2% 2-fluoro-ara-adenine; and
not more than 2.0% of any other late-eluting impurity.

13. The pharmaceutical product of claim 1, wherein after storage at 25° C. and 60% relative humidity for 2 weeks, the composition includes:
not more than 1.0% iso-ara-guanine-monophosphate;
not more than 0.2% isoguanine;
not more than 0.2% of a 3,5-diphosphate analog;
not more than 0.2% of any other early-eluting impurity;
not more than 0.2% 2-fluoroadenine;
not more than 0.2% 2-fluoro-ara-adenine;
not more than 1.0% of a 2-ethoxyphosphate analog; and
not more than 2.0% of any other late-eluting impurity.

14. The pharmaceutical product of claim 1, wherein after storage at 40° C. and 75% relative humidity for 2 weeks, the composition includes:
not more than 1.0% iso-ara-guanine-monophosphate;
not more than 0.2% isoguanine;
not more than 0.2% of a 3,5-diphosphate analog;
not more than 0.2% of any other early-eluting impurity;
not more than 0.2% 2-fluoroadenine;
not more than 0.2% 2-fluoro-ara-adenine;
not more than 1.0% of a 2-ethoxyphosphate analog; and
not more than 2.0% of any other late-eluting impurity.

15. The pharmaceutical product of claim 1, wherein after storage at 2-8° C. for 4 weeks, the composition includes:
not more than 1.0% iso-ara-guanine-monophosphate;
not more than 0.2% isoguanine;
not more than 0.2% of a 3,5-diphosphate analog;
not more than 0.2% of any other early-eluting impurity;
not more than 0.2% 2-fluoroadenine;
not more than 0.2% 2-fluoro-ara-adenine;
not more than 1.0% of a 2-ethoxyphosphate analog; and
not more than 2.0% of any other late-eluting impurity.

16. The pharmaceutical product of claim 1, wherein the product is prepared by a method comprising:
purging an amount of water for injection representing about 80% of a desired total composition volume with nitrogen gas;
heating the purged water for injection to about 70° C.;
adding methyl paraben and/or propyl paraben to the heated water for injection under continuous stirring;
cooling the mixture of water for injection and methyl paraben and/or propyl paraben to room temperature;
adding mannitol and/or sorbitol to the room temperature mixture under continuous stirring;
adding fludarabine phosphate to the mixture of methyl paraben and/or propyl paraben, mannitol and/or sorbitol, and water for injection under continuous stirring;
adding, if needed, an amount of sodium hydroxide to bring a pH of the fludarabine phosphate mixture to 6.8;
adding, if needed, additional water for injection to achieve the desired total composition volume;
stirring the mixture at the desired total composition volume;
placing the stirred mixture into one or more storage containers; and
sealing the storage containers under nitrogen blanketing.

17. The pharmaceutical product of claim 16 further comprising sterilizing the stirred mixture before the step of placing the stirred mixture into one or more storage containers.

18. The pharmaceutical product of claim 16 further comprising sterilizing the stirred mixture after the step of sealing the storage containers under nitrogen blanketing.

19. The pharmaceutical product of claim 1, wherein the multi-use container is a glass vial.

20. A pharmaceutical product comprising:
a multi-use container; and
a sterile composition housed within the multi-use container, the composition including:
fludarabine phosphate in an amount selected from the group consisting of: 25 mg/mL and 30 mg/mL,
sorbitol in an amount selected from the group consisting of: 0 mg/mL, 10 mg/mL, 20 mg/mL, and 40 mg/mL,
0 mg/mL mannitol,
1.8 mg/mL methyl paraben,
0.2 mg/mL propyl paraben,
0 mg/mL benzyl alcohol,
water for injection,
a pH adjuster in an amount to provide an initial pH level of 6.5 to 7.1, and optionally an inert gaseous atmosphere,
wherein after storage in the multi-use container, the sterile composition has one or more characteristic selected from the group consisting of:
a pH level within 0.5 pH units of the initial pH level,
not more than 0.5% iso araguanine monophosphate,
not more than 0.5% iso guanine,
not more than 0.5% 3,5-diphosphate analog,
not more than 0.5% 2-fluoroadenine,
not more than 0.5% 2-fluoroara adenine,
not more than 0.5% 2-ethoxyphosphate analog, and
not more than 1% total impurities.

21. The pharmaceutical product of claim 20, wherein the storage is at 2-8° C. for at least 3 months, at 25° C. and 60% relative humidity for at least 3 months, or at 40° C. and 75% relative humidity for at least 2 weeks.

22. The pharmaceutical product of claim 20, wherein the multi-use container is a glass vial.
